(19) United States
(12) Patent Application Publication
(10) Pub. No.: US 2024/0417446 A1
(43) Pub. Date: Dec. 19, 2024

(54) SINGLE DOMAIN ANTIBODIES TARGETING THE S2 SUBUNIT OF SARS-COV-2 SPIKE PROTEIN

(71) Applicant: The U.S.A., as represented by the Secretary, Department of Health and Human Services, Bethesda, MD (US)

(72) Inventors: Mitchell Ho, Urbana, MD (US); Zhijian Duan, Urbana, MD (US); Jesse D. Buffington, Rockville, MD (US)

(73) Assignee: The U.S.A., as represented by the Secretary, Department of Health and Human Services, Bethesda, MD (US)

(21) Appl. No.: 18/704,211

(22) PCT Filed: Oct. 25, 2022
(86) PCT No.: PCT/US2022/078632

§ 371 Date: Apr. 24, 2024

Related U.S. Application Data
(60) Provisional application No. 63/271,854, filed on Oct. 26, 2021.

(51) Int. Cl. C07K 16/10; G01N 33/569

(52) U.S. Cl. CPC C07K 16/1003; G01N 33/56983; C07K 2317/569; C07K 2317/76; C07K 2317/92; G01N 2333/165; G01N 2469/10

(57) ABSTRACT

Single-domain monoclonal antibodies (“nanobodies”) that specifically bind the S2 subunit of the severe acute respiratory syndrome coronavirus 2 (SARS-CoV-2) spike protein are described. The single-domain antibodies were isolated from shark variable domain of new antigen receptor (V_{NAR}) and camel variant domain of heavy chain only antibody (V_{H}H) phage display libraries panned against the S2 subunit of SARS-CoV-2 spike protein. The S2 subunit-specific nanobodies, and conjugates thereof, can be used for the diagnosis and treatment of a coronavirus infection.

### CROSS REFERENCE TO RELATED APPLICATION

This application claims the benefit of U.S. Provisional Application No. 63/271,854, filed Oct. 26, 2021, which is herein incorporated by reference in its entirety.

### ACKNOWLEDGMENT OF GOVERNMENT SUPPORT

This invention was made with government support under project numbers Z01 BC010891 and ZIA BC 011943 awarded by the National Institutes of Health. The government has certain rights in the invention.

### FIELD

This disclosure concerns shark and camel single-domain monoclonal antibodies that specifically bind the S2 subunit of the severe acute respiratory syndrome coronavirus 2 (SARS-CoV-2) spike protein and their use, such as for the detection and treatment of a coronavirus infection.

### BACKGROUND

The coronavirus disease 2019 (COVID-19) pandemic caused by SARS-CoV-2 has infected over 240 million people and led to more than 4 million deaths worldwide. The virus spike trimer protein has S1 and S2 subunits. The S1 subunit is located within the N-terminal 14-685 amino acids of the spike protein, which includes the N-terminal domain (NTD) and receptor binding domain (RBD). The RBD binds to angiotensin converting enzyme-2 (ACE2) on human cells, which is the first step in SARS-CoV-2 infection. The S2 subunit mediates membrane fusion of the virus with host cells; it contains the fusion peptide (FP), heptad repeat 1 (HR1), heptad repeat 2 (HR2), transmembrane Domain™ and cytoplasmic domain (CP). After binding of the S1 subunit RBD to ACE2 on a target cell, proteolytic cleavage of a N-terminal fragment occurs, which is mediated by transmembrane serine protease 2 (TMPRSS2) in each S2 monomer. This cleavage triggers a series of conformational changes in S2. Subsequently, the HR1 and HR2 domains in the trimer interact with each other to form a six-helix bundle (6-HB) fusion core, bringing viral and cellular membranes into close proximity for fusion and infection, thereby delivering the genetic payload of the virus into the cytoplasm. The S1 subunit, in particular its RBD, has been the dominant target for developing neutralizing antibodies to treat COVID-19. However, there is a high frequency of mutations in the S1 subunit that result in the emergence of SARS-CoV-2 variants including B.1.1.7 (Alpha, UK), B.1.351 (Beta, South Africa or SA), P.1 (Gamma, Brazil), and B.1.617.2 (Delta, India). These variants cause a reduction in protection conferred by neutralizing antibodies developed or elicited by vaccination against the reference strain. Thus, there remains an urgent need for effective therapies for COVID-19, especially to treat disease caused by emerging SARS-CoV-2 variants.

### SUMMARY

Disclosed herein are single-domain monoclonal antibodies (“nanobodies”) that specifically bind the S2 subunit of the SARS-CoV-2 spike protein. The single-domain antibodies were isolated from shark (V_{N}AR) and camel (V_{H}H) antibody libraries. Nanobodies specific for the S2 subunit of the spike protein disrupt fusion of the virus with host membranes, thereby blocking an important step for coronavirus infection of human cells. Use of the disclosed single-domain monoclonal antibodies for the detection, diagnosis and treatment of SARS-CoV-2 and/or SARS-CoV is described. Due to their small size and high stability, the disclosed nanobodies are suitable for administration via inhalation, such as with an inhaler. The disclosed nanobodies can also be administered orally using bacteria or yeast engineered to express a nanobody disclosed herein.

Provided herein are polypeptides (for example, single-domain monoclonal antibodies) that bind, such as specifically bind, the S2 subunit of a coronavirus (such as SARS-CoV-2 or a variant thereof) spike protein. In some aspects, the polypeptide includes the complementarity determining region (CDR) sequences of shark nanobody NCI-CoV-S2A9 (S2A9), NCI-CoV-S2G8 (S2G8), NCI-CoV-S3A10 (S3A10), NCI-CoV-S4A9 (S4A9), NCI-CoV-S4C12 (S4C12) or NCI-CoV-S6E1 (S6E1), or camel nanobody NCI-CoV-CLA2 (CLA2), NCI-CoV-CG2G2 (CG2G2), NCI-CoV-CGH3 (CGH3), NCI-CoV-CGF10 (CGF10), NCI-CoV-CG2D11 (CG2D11) or NCI-CoV-CL3H4 (CL3H4). Also provided herein are conjugates that include a disclosed polypeptide. In some examples, provided are fusion proteins (such as Fc fusion proteins), chimeric antigen receptors (CARs), CAR-expressing immune cells (such as T cells, B cells, natural killer cells, macrophages and induced pluripotent stem cells (iPSCs)), immunoconjugates (such as immunotoxins), multi-specific antibodies (such as bispecific or trispecific antibodies), antibody-drug conjugates (ADCs), antibody-nanoparticle conjugates, and antibody-radioisotope conjugates (such as for immunoPET imaging) that include a polypeptide (for example, single-domain monoclonal antibody) disclosed herein. In some examples, a disclosed nanobody is converted to an IgM or IgA molecule, such as for mucosal administration.

Further provided are compositions that include one or more (such as at least two, at least three, at least four or at least five) different S2 subunit-specific polypeptides disclosed herein. In some examples, such a composition includes a pharmaceutically acceptable carrier. In some examples, such a composition is lyophilized.

Also provided herein are nucleic acid molecules and vectors encoding the S2 subunit-specific polypeptides (for example, antibodies), fusion proteins (such as Fc fusions or nanobodies converted to IgG, IgM or IgA), CARs, immunoconjugates (such as immunotoxins), and multi-specific antibodies (such as bi-specific antibodies) disclosed herein. Isolated cells that include a nucleic acid or vector encoding an S2 subunit-specific polypeptide or CAR are further provided.

Compositions that include a pharmaceutically acceptable carrier and an S2 subunit-specific polypeptide, fusion protein, CAR, CAR-expressing cell, immunoconjugate, ADC, multi-specific antibody, antibody-nanoparticle conjugate, isolated nucleic acid molecule or vector disclosed herein are also provided by the present disclosure. Also provided are solid supports, such as beads (e.g., glass, magnetic, plastic), multiwell plates, paper, or nitrocellulose that include one or more S2 subunit-specific polypeptides (such as single-domain monoclonal antibodies) provided herein. In some aspects, the compositions are formulated for administration via intranasal spray. In some aspects, the composition includes recombinant yeast or bacteria that express a disclosed antibody.

Also provided are solid supports that include one or more of the polypeptides disclosed herein. In some aspects, the solid support includes a bead, multiwell plate, or nitrocellulose. Use of the solid support for detecting a coronavirus in a sample is also provided.

Methods of detecting a coronavirus in a sample, and methods of diagnosing a subject as having a coronavirus infection, are further provided. In some aspects, the methods include contacting a sample obtained from the subject with a polypeptide (for example, a single-domain monoclonal antibody) disclosed herein, and detecting binding of the polypeptide to the sample. In specific examples, the coronavirus is SARS-CoV-2, a SARS-CoV-2 variant (including any variant thereof,) or SARS-CoV, such as one or more of SARS-CoV-2 (“Wildtype”); SARS-CoV-2 alpha variant; SARS-CoV-2 beta variant; SARS-CoV-2 delta variant; SARS-CoV-2 gamma variant; SARS-CoV-2 lambda variant; SARS-CoV-2 omicron BA.1 variant; SARS-CoV-2 omicron BA.2 variant; SARS-CoV-2 omicron BA.4/BA.5; and SARS-CoV-1.

Also provided is a method of treating a coronavirus infection in a subject. In some aspects, the method includes administering to the subject a therapeutically effective amount of a polypeptide (for example, a single-domain monoclonal antibody) disclosed herein, or administering to the subject a therapeutically effective amount of a fusion protein, CAR (or CAR T cells, CAR B cells, CAR NK cells, CAR macrophages or CAR iPSCs), immunoconjugate (such as an immunotoxin), ADC, multi-specific antibody, or antibody-nanoparticle conjugate comprising a polypeptide disclosed herein, or a nucleic acid molecule or vector encoding a disclosed polypeptide. In some examples, administration is via inhalation. In specific examples, the coronavirus is SARS-CoV-2 (including any variant thereof, such as SARS-CoV-2 alpha variant; SARS-CoV-2 beta variant; SARS-CoV-2 delta variant; SARS-CoV-2 gamma variant; SARS-CoV-2 lambda variant; SARS-CoV-2 omicron BA.1 variant; SARS-CoV-2 omicron BA.2 variant; or SARS-CoV-2 omicron BA.4/BA.5) or SARS-CoV. Such treatments can be combined with other therapeutic agents, such as other anti-viral therapies.

The foregoing and other objects and features of the disclosure will become more apparent from the following detailed description, which proceeds with reference to the accompanying figures.

### BRIEF DESCRIPTION OF THE DRAWINGS

FIG. 1: Schematic showing the procedure for isolation of shark and camel neutralizing nanobodies from phage display libraries panned against the S2 subunit of SARS-CoV-2 spike protein.

FIGS. 2A-2B: Enrichment of S2-specific phage by polyclonal phage ELISA. Phage panning was performed with shark (FIG. 2A) and camel (FIG. 2B) single domain libraries against the S2 subunit of the SARS-CoV-2 spike protein for one round. Eluted phages were amplified and panned against the same antigen for an additional two (shark) or three (camel) rounds.

FIGS. 3A-3D: Protein ELISA of top binders. Antigen (2 mg/ml) was coated on an ELISA plate and the wells were incubated with different concentrations of shark nanobody NCI-CoV-S2A9 (FIG. 3A) or NCI-CoV-S2G8 (FIG. 3B), or camel nanobody NCI-CoV-CLA2 (FIG. 3C) or NCI-CoV-CG2G2 (FIG. 3D). Secondary HRP-anti-Flag antibody was added and OD₄₅₀ was read by a plate reader after development with the TMB substrate.

FIGS. 4A-4B: Antibody cross-binding to S2 subunits of different coronaviruses. S2 subunits from SARS-CoV-2, SARS-CoV, MERS or human coronavirus 43 (HCoV-OC43) were coated on wells of an ELISA plate. The wells were incubated with different concentrations of shark nanobody NCI-CoV-S2A9 or NCI-CoV-S2G8 (FIG. 4A), or camel nanobody NCI-CoV-CLA2 or NCI-CoV-CG2G2 (FIG. 4B). Secondary HRP-anti-Flag antibody was added and OD₄₅₀ was read by a plate reader after development with the TMB substrate.

FIGS. 5A-5D: Neutralization measured by pseudovirus assays. (FIGS. 5A and 5C) 293-hACE2 cells were plated. Pseudovirus expressing SARS-CoV-2 spike protein was incubated with the indicated antibodies (FIG. 5A) or Fc fusions (FIG. 5C) at a concentration of 200 mg/ml. The luciferase signal was read by plate reader to calculate the inhibitory activity. (FIGS. 5B and 5D) The IC₅₀ of shark NCI-CoV-S2A9-V_{NAR} (FIG. 5B) and NCI-CoV-SA29-hFc (FIG. 5D) was determined based on inhibitory activity from various antibody concentrations.

FIGS. 6A-6D: Neutralization of SARS-CoV-2 Delta variant. (FIGS. 6A and 6C) 293T cells overexpressing hACE2 were plated. Pseudovirus expressing SARS-CoV-2 delta variant spike protein were incubated with the single domain format of S2A9 (S2A9-6×His-FLAG; FIG. 6A) and bivalent S2A9 with a human Fc tag (S2A9-hFc; FIG. 6C) at the indicated concentrations. The luciferase signal was read by plate reader to calculate the inhibitory activity. The IC₅₀ of shark S2A9-6×His-FLAG (FIG. 6B) and S2A9-hFc (FIG. 6D) was determined based on inhibitory activity from various antibody concentrations.

FIGS. 7A-7J: Inhibition of SARS-CoV-2, SARS-CoV-2 variant and SARS-CoV-1 pseudoviruses by S2A9 nanobody and S2A9-Fc. Shown is inhibition of the following pseudoviruses: (FIG. 7A) SARS-CoV-2 (“Wildtype”); (FIG. 7B) Alpha variant; (FIG. 7C) Beta variant; (FIG. 7D) Delta variant; (FIG. 7E) Gamma variant; (FIG. 7F) Lambda variant; (FIG. 7G) Omicron BA.1 variant; (FIG. 7H) Omicron BA.2 variant; (FIG. 7I) Omicron BA.4/BA.5; and (FIG. 7J) SARS-CoV-1.

FIGS. 8A-8B: Affinity of S2A9 (FIG. 8A) and CG2G (FIG. 8B) for binding to the S2 subunit of spike protein.

FIG. 9: Table summarizing binding characteristics and neutralization capacity of the S2-specific shark and camel nanobodies.

### SEQUENCE LISTING

The Sequence Listing is submitted as an ST.26 Sequence Listing XML file, named 4239-107163-02.xml, created on Oct. 14, 2022, having a size of 38,999 bytes, which is incorporated by reference herein. In the accompanying sequence listing:

SEQ ID NO: 1 is the amino acid sequence of shark VNAR NCI-CoV-S2A9.

SEQ ID NO: 2 is the amino acid sequence of shark VNAR NCI-CoV-S2G8.

SEQ ID NO: 3 is the amino acid sequence of shark VNAR NCI-CoV-S3A10.

SEQ ID NO: 4 is the amino acid sequence of shark VNAR NCI-CoV-S4A9.

SEQ ID NO: 5 is the amino acid sequence of shark VNAR NCI-CoV-S4C12.

SEQ ID NO: 6 is the amino acid sequence of shark VNAR NCI-CoV-S6E1.

SEQ ID NO: 7 is the amino acid sequence of camel VHH NCI-CoV-CLA2.

SEQ ID NO: 8 is the amino acid sequence of camel VHH NCI-CoV-CG2G2.

SEQ ID NO: 9 is the amino acid sequence of camel VHH NCI-CoV-CGH3.

SEQ ID NO: 10 is the amino acid sequence of camel VHH NCI-CoV-CGF10.

SEQ ID NO: 11 is the amino acid sequence of camel VHH NCI-CoV-CG2D11.

SEQ ID NO: 12 is the amino acid sequence of camel VHH NCI-CoV-CL3H4.

SEQ ID NO: 13 is a nucleic acid sequence encoding shark VNAR NCI-CoV-S2A9.

SEQ ID NO: 14 is a nucleic acid sequence encoding shark VNAR NCI-CoV-S2G8.

SEQ ID NO: 15 is a nucleic acid sequence encoding shark VNAR NCI-CoV-S3A10.

SEQ ID NO: 16 is a nucleic acid sequence encoding shark VNAR NCI-CoV-S4A9.

SEQ ID NO: 17 is a nucleic acid sequence encoding shark VNAR NCI-CoV-S4C12.

SEQ ID NO: 18 is a nucleic acid sequence encoding shark VNAR NCI-CoV-S6E1.

SEQ ID NO: 19 is a nucleic acid sequence encoding camel VHH NCI-CoV-CLA2.

SEQ ID NO: 20 is a nucleic acid sequence encoding camel VHH NCI-CoV-CG2G2.

SEQ ID NO: 21 is a nucleic acid sequence encoding camel VHH NCI-CoV-CGH3.

SEQ ID NO: 22 is a nucleic acid sequence encoding camel VHH NCI-CoV-CGF10.

SEQ ID NO: 23 is a nucleic acid sequence encoding camel VHH NCI-CoV-CG2D11.

SEQ ID NO: 24 is a nucleic acid sequence encoding camel VHH NCI-CoV-CL3H4.

SEQ ID NO: 25 is the amino acid sequence of a shark VNAR HV2.

SEQ ID NO: 26 is the amino acid sequence of a shark VNAR HV4.

### DETAILED DESCRIPTION

### I. Abbreviations

ACE2 angiotensin converting enzyme 2
ADC antibody-drug conjugate
CAR chimeric antigen receptor
CDR complementarity determining region
CoV coronavirus
COVID-19 coronavirus disease 2019
ELISA enzyme-linked immunosorbent assay
FR framework
hFc human Fc
HV hypervariable
IC₅₀ inhibitory concentration 50
MERS Middle East respiratory syndrome coronavirus
NK natural killer
NTD N-terminal domain
PE Pseudomonas exotoxin
PET positron emission tomography
RBD receptor binding domain
S spike protein
SARS severe acute respiratory syndrome
V_{NAR} variable new antigen receptor

### II. Terms and Methods

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references.

As used herein, the singular forms “a,” “an,” and “the,” refer to both the singular as well as plural, unless the context clearly indicates otherwise. For example, the term “an antigen” includes single or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting.

To facilitate review of the various aspects, the following explanations of terms are provided:

Administration: To provide or give a subject an agent, such as a polypeptide (e.g., single domain monoclonal antibody) provided herein, by any effective route. Exemplary routes of administration include, but are not limited to, oral, injection (such as subcutaneous, intramuscular, intradermal, intraperitoneal, intravenous, and intratumoral), sublingual, rectal, transdermal, intranasal, vaginal and inhalation routes.

Antibody: A polypeptide ligand comprising at least one variable region that recognizes and binds (such as specifically recognizes and specifically binds) an epitope of an antigen. Mammalian immunoglobulin molecules are composed of a heavy (H) chain and a light (L) chain, each of which has a variable region, termed the variable heavy (V_{H}) region and the variable light (V_{L}) region, respectively. Together, the V_{H} region and the V_{L} region are responsible for binding the antigen recognized by the antibody. There are five main heavy chain classes (or isotypes) of mammalian immunoglobulin, which determine the functional activity of an antibody molecule: IgM, IgD, IgG, IgA and IgE. Antibody isotypes not found in mammals include IgX, IgY, IgW and IgNAR. IgY is the primary antibody produced by birds and reptiles, and is functionally similar to mammalian IgG and IgE. IgW and IgNAR antibodies are produced by cartilaginous fish, while IgX antibodies are found in amphibians.

Antibody variable regions contain framework regions (FR) and hypervariable (HV) regions, known as “complementarity determining regions” or “CDRs.” The CDRs are primarily responsible for binding to an epitope of an antigen. The framework regions of an antibody serve to position and align the CDRs in three-dimensional space. The amino acid sequence boundaries of a given CDR can be readily determined using any of a number of numbering schemes, including those described by Kabat et al. (Sequences of Proteins of Immunological Interest, U.S. Department of Health and Human Services, 1991; the “Kabat” numbering scheme), Chothia et al. (see Chothia and Lesk, J Mol Biol 196:901-917, 1987; Chothia et al., Nature 342:877, 1989; and Al-Lazikani et al., JMB 273,927-948, 1997; the “Chothia” numbering scheme), Kunik et al. (see Kunik et al., PLoS Comput Biol 8:e1002388, 2012; and Kunik et al., Nucleic Acids Res 40(Web Server issue):W521-524, 2012; “Paratome CDRs”) and the ImMunoGeneTics (IMGT) database (see, Lefranc, Nucleic Acids Res 29:207-9, 2001; the “IMGT” numbering scheme). The Kabat, Paratome and IMGT databases are maintained online.

A “single-domain antibody” refers to an antibody having a single domain (a variable domain) that is capable of specifically binding an antigen, or an epitope of an antigen, in the absence of an additional antibody domain. Single-domain antibodies include, for example, V_{H} domain antibodies, V_{NAR} antibodies, camelid V_{H}H antibodies, and V_{L} domain antibodies. V_{NAR} antibodies are produced by cartilaginous fish, such as nurse sharks, wobbegong sharks, spiny dogfish and bamboo sharks. Shark V_{NAR} are comprised of the following regions (N-terminal to C-terminal): FR1-CDR1-FR2-HV2-FR3a-HV4-FR3b-CDR3-FR4. The positions of CDR1 and CDR3 of V_{NAR} antibodies can be determined, for example, using IMGT. HV2 and HV4 can be determined, for example, using annotation described in Stanfield et al. (Science 305:1770-1773, 2004) and Fennell et al. (J Mol Biol 400:155-170, 2010). Camelid V_{H}H antibodies are produced by several species including camel, llama, alpaca, dromedary, and guanaco, which produce heavy chain antibodies that are naturally devoid of light chains. Camel V_{H}H are comprised of the following regions (N-terminal to C-terminal): FR1-CDR1-FR2-CDR2-FR3-CDR3-FR4. Camel V_{H}H CDR residues can be determined, for example, according to IMGT, Kabat or Paratome.

A “monoclonal antibody” is an antibody produced by a single clone of lymphocytes or by a cell into which the coding sequence of a single antibody has been transfected. Monoclonal antibodies include humanized monoclonal antibodies.

A “chimeric antibody” has framework residues from one species, such as human, and CDRs (which generally confer antigen binding) from another species.

A “humanized” antibody is an immunoglobulin including a human framework region and one or more CDRs from a non-human (for example a mouse, rabbit, rat, shark or synthetic) immunoglobulin. The non-human immunoglobulin providing the CDRs is termed a “donor,” and the human immunoglobulin providing the framework is termed an “acceptor.” In one aspect, all CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A humanized antibody binds to the same antigen as the donor antibody that provides the CDRs. Humanized or other monoclonal antibodies can have additional conservative amino acid substitutions which have substantially no effect on antigen binding or other immunoglobulin functions.

Antibody-drug conjugate (ADC): A molecule that includes an antibody (or antigen-binding fragment of an antibody) conjugated to a drug, such as an anti-viral agent or a cytotoxic agent. ADCs can be used to specifically target a drug to particular cells through specific binding of the antibody to a target antigen expressed on the cell surface. Exemplary drugs for use with ADCs include anti-viral agents (such as remdesivir, galidesivir, arbidol, favipiravir, baricitinib, or lopinavir/ritonavir), anti-microtubule agents (such as maytansinoids, auristatin E and auristatin F) and interstrand crosslinking agents (for example, pyrrolobenzodiazepines; PBDs). In some cases, the ADC is a bi-specific ADC, which is comprised of two monoclonal antibodies or antigen-fragments thereof, each directed to a different antigen or epitope, conjugated to a drug. In one example, the agent attached to the antibody is IRDye® 700 DX (IR700, Li-cor, Lincoln, NE), which can then be used with near infrared light NIR light to kill target cells to which the antibody binds (photoimmunotherapy; see for example U.S. Pat. Nos. 8,524,239 and 10,538,590). For example, amino-reactive IR700 can be covalently conjugated to an antibody using the NHS ester of IR700.

Binding affinity: Affinity of an antibody for an antigen. In one aspect, affinity is calculated by a modification of the Scatchard method described by Frankel et al., Mol. Immunol., 16:101-106, 1979. In another aspect, binding affinity is measured by an antigen/antibody dissociation rate. In another aspect, a high binding affinity is measured by a competition radioimmunoassay. In another aspect, binding affinity is measured by ELISA. In some aspects, binding affinity is measured using the Octet system (Creative Biolabs), which is based on bio-layer interferometry (BLI) technology. In other aspects, Kd is measured using surface plasmon resonance assays using a BIACORES-2000 or a BIACORES-3000 (BIAcore, Inc., Piscataway, N.J.). In other aspects, antibody affinity is measured by flow cytometry or by surface plasmon reference. An antibody that “specifically binds” an antigen (such as CoV spike protein) is an antibody that binds the antigen with high affinity and does not significantly bind other unrelated antigens.

In some examples, a single-domain monoclonal antibody (such as an anti-S2 single-domain antibody provided herein) specifically binds to a target (for example, a CoV spike protein, such as a S2 subunit of the SARS-CoV S protein, S2 subunit of the SARS-CoV-2 S protein, or to both the S2 subunit of the SARS-CoV-2 S protein and the S2 subunit of the SARS-CoV S protein) with a binding constant that is at least 10³ M⁻¹ greater, 10⁴M⁻¹ greater or 10⁵ M⁻¹ greater than a binding constant for other molecules in a sample or subject. In some examples, an antibody (e.g., monoclonal antibody) has an equilibrium constant (Kd) of 10 nM or less, such as 9 nM or less, 8 nM or less, 7 nM or less, 6 nM or less, 5.7 nM or less, 5.5 nM or less, 5.3 nM or less, 5 nM or less, 4.3 nM or less, 4 nM or less, 3 nM or less, 2 nM or less, 1.5 nM or less, 1.5 nM or less, 1.4 nM or less, 1.3 nM or less, or 1.2 nM or less. For example, a monoclonal antibody binds to a target, such as the S2 subunit of the CoV S protein (such as the S2 subunit of the SARS-CoV S protein, the S2 subunit of the SARS-CoV-2 S protein, or to both the S2 subunit of the SARS-CoV-2 S protein and the S2 subunit of the SARS-CoV S protein) with a binding affinity of at least about 0.1×10⁻⁸ M, at least about 0.3×10⁻⁸ M, at least about 0.5×10⁻⁸ M, at least about 0.75×10⁻⁸ M, at least about 1.0×10⁻⁸ M, at least about 1.3×10⁻⁸ M at least about 1.5×10⁻⁸ M, or at least about 2.0×10⁻⁸ M, at least about 2.5×10⁻⁸, at least about 3.0×10⁻⁸, at least about 3.5×10⁻⁸, at least about 4.0×10⁻⁸, at least about 4.5×10⁻⁸, at least about 5.0×10⁻⁸ M, at least about 1×10⁻⁹ M, at least about 1.3×10⁻⁹ M, at least about 1.5×10⁻⁹ M, at least about 2×10⁻⁹ M, at least about 3×10⁻⁹ M, at least about 4×10⁻⁹ M, at least about 4.3×10⁻⁹ M, at least about 5×10⁻⁹ M, at least about 6×10⁻⁹ M, at least about 6.3×10⁻⁹ M, at least about 6.9×10⁻⁹ M, at least about 7×10⁻⁹ M, at least about 8×10⁻⁹ M, at least about 8.1×10⁻⁹ M, or at least about 10×10⁻⁹ M. In certain aspects, a specific binding agent that binds to its target has a dissociation constant (Kd) of ≤1 μM, ≤100 nM, ≤10 nM, ≤9 nM, ≤8 nM, ≤7 nM, ≤6.9 nM, ≤6.5 nM, ≤6.3 nM, ≤5 nM, ≤4 nM, ≤4.5 nM, ≤3 nM, ≤2 nM, ≤1.5 nM, ≤1 nM, ≤0.1 nM, ≤0.01 nM, or ≤0.001 nM (e.g., 10⁻⁸ M or less, e.g., from 10⁻⁸ M to 10⁻¹³M, e.g., from 10⁻⁹ M to 10⁻¹³ M).

Bispecific antibody: A recombinant protein that includes antigen-binding fragments of two different monoclonal antibodies and is thereby capable of binding two different antigens, such as the S2 subunit of the SARS-CoV-2 S protein and the S1 subunit of the SARS-CoV-2 S protein. Similarly, a multi-specific antibody is a recombinant protein that includes antigen-binding fragments of at least two different monoclonal antibodies, such as two, three or four different monoclonal antibodies.

Chimeric antigen receptor (CAR): A chimeric molecule that includes an antigen-binding portion (such as single-domain antibody) and a signaling domain, such as a signaling domain from a T cell receptor (for example, CD3ζ). Typically, CARs are comprised of an antigen-binding moiety, a transmembrane domain and an endodomain. The endodomain typically includes a signaling chain having an immunoreceptor tyrosine-based activation motif (ITAM), such as CD3ζ or FcERIγ. In some instances, the endodomain further includes the intracellular portion of at least one additional co-stimulatory domain, such as CD28, 4-1BB (CD137), ICOS, OX40 (CD134), CD27, MYD88-CD40, KIR2DS2 and/or DAP10. In some examples, the CAR is multispecific (such as bispecific) or bicistronic. A multispecific CAR is a single CAR molecule comprised of at least two antigen-binding domains (such as scFvs and/or single-domain antibodies) that each bind a different antigen or a different epitope on the same antigen (see, for example, US 2018/0230225). For example, a bispecific CAR refers to a single CAR molecule having two antigen-binding domains that each bind a different antigen. A bicistronic CAR refers to two complete CAR molecules, each containing an antigen-binding moiety that binds a different antigen. In some cases, a bicistronic CAR construct expresses two complete CAR molecules that are linked by a cleavage linker. T cells or NK cells (or other immune cells, such as macrophages) expressing a bispecific or bicistronic CAR can bind cells that express both of the antigens to which the binding moieties are directed (see, for example, Qin et al., Blood 130:810, 2017; and WO/2018/213337). In some aspects, the CAR is a two-chained antibody-T cell receptor (AbTCR) as described in Xu et al. (Cell Discovery 4:62, 2018) or a synthetic T cell receptor and antigen receptor (STAR) as described by Liu et al. (Sci Transl Med 13(586):eabb5191, 2021).

Complementarity determining region (CDR): A region of hypervariable amino acid sequence that defines the binding affinity and specificity of an antibody. The light and heavy chains of a mammalian immunoglobulin each have three CDRs, designated L-CDR1, L-CDR2, L-CDR3 and H-CDR1, H-CDR2, H-CDR3, respectively. Camel (V_{H}H) single-domain antibodies, VH single-domain antibodies and VL single-domain antibodies contain three CDRs, referred to as CDR1, CDR2 and CDR3. Shark VNAR single-domain antibodies include two CDRs (CDR1 and CDR3) as well as two hypervariable regions (HV2 and HV4).

Conjugate: In the context of the present disclosure, a “conjugate” is an antibody or antibody fragment (such as an antigen-binding fragment) covalently linked to an effector molecule or a second protein (such as a second antibody). The effector molecule can be, for example, a drug, toxin, therapeutic agent, detectable label, protein, nucleic acid, lipid, nanoparticle, photon absorber, carbohydrate or recombinant virus. An antibody conjugate is often referred to as an “immunoconjugate.” When the conjugate includes an antibody linked to a drug (such as a cytotoxic agent), the conjugate is often referred to as an “antibody-drug conjugate” or “ADC.” Other antibody conjugates include, for example, multi-specific (such as bispecific or trispecific) antibodies and chimeric antigen receptors (CARs).

Conservative variant: A protein containing conservative amino acid substitutions that do not substantially affect or decrease the affinity of a protein, such as an antibody to a spike (S) protein (such as an S2 subunit of the SARS-CoV S protein, SARS-CoV-2 S protein, or to both an S2 subunit of the SARS-CoV-2 S protein and an S2 subunit of the SARS-CoV S protein). For example, a monoclonal antibody that specifically binds to an S2 subunit of the S protein can include at most about 1, at most about 2, at most about 5, and most about 10, or at most about 15 conservative substitutions and specifically bind the S2 subunit of the S protein. The term “conservative variant” also includes the use of a substituted amino acid in place of an unsubstituted parent amino acid, provided that antibody specifically binds the S2 subunit of the S protein. Non-conservative substitutions are those that reduce an activity or binding to the S2 subunit of the S protein.

Conservative amino acid substitution tables providing functionally similar amino acids are well known. The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Contacting: Placement in direct physical association; includes both in solid and liquid form.

Coronavirus: A large family of positive-sense, single-stranded RNA viruses that can infect humans and non-human animals. Coronaviruses get their name from the crown-like spikes on their surface.

The viral envelope is comprised of a lipid bilayer containing the viral membrane (M), envelope (E) and spike (S) proteins. Most coronaviruses cause mild to moderate upper respiratory tract illness, such as the common cold. However, three coronaviruses have emerged that can cause more serious illness and death: severe acute respiratory syndrome coronavirus (SARS-CoV), SARS-CoV-2 (including variants thereof, such as: alpha (B.1.1.7 and Q lineages); beta (B.1.351 and descendent lineages); delta (B.1.617.2 and AY lineages); gamma (P.1 and descendent lineages); epsilon (B.1.427 and B.1.429); eta (B.1.525); iota (B.1.526); kappa (B.1.617.1); 1.617.3; mu (B.1.621, B.1.621.1); zeta (P.2); and omicron (B.1.1.529, BA.1, BA.1.1, BA.2, BA.3, BA.4 and BA.5 lineages)), and Middle East respiratory syndrome coronavirus (MERS-CoV). Other coronaviruses that infect humans include human coronavirus HKU1 (HKU1-CoV), human coronavirus OC43 (OC43-CoV), human coronavirus 229E (229E-CoV), human coronavirus NL63 (NL63-CoV).

COVID-19: The disease caused by the coronavirus SARS-CoV-2.

Cytotoxic agent: Any drug or compound that kills cells.

Cytotoxicity: The toxicity of a molecule, such as an immunotoxin, to the cells intended to be targeted, as opposed to the cells of the rest of an organism. In contrast, the term “toxicity” refers to toxicity of an immunotoxin to cells other than those that are the cells intended to be targeted by the targeting moiety of the immunotoxin, and the term “animal toxicity” refers to toxicity of the immunotoxin to an animal by toxicity of the immunotoxin to cells other than those intended to be targeted by the immunotoxin.

Degenerate variant: A polynucleotide encoding a polypeptide that includes a sequence that is degenerate as a result of the genetic code. There are 20 natural amino acids, most of which are specified by more than one codon. Therefore, all degenerate nucleotide sequences are included as long as the amino acid sequence of the polypeptide is unchanged.

Detect: To determine if a particular agent or analyte is present or absent, and in some examples further includes quantification of the agent/analyte if detected. In some examples, the agent detected is a coronavirus, such as SARS-CoV and/or SARS-CoV-2.

Diagnostic: Identifying the presence or nature of a pathologic condition, such as a coronavirus infection. Diagnostic methods differ in their sensitivity and specificity. The “sensitivity” of a diagnostic assay is the percentage of diseased individuals who test positive (percent of true positives). The “specificity” of a diagnostic assay is one minus the false positive rate, where the false positive rate is defined as the proportion of those without the disease who test positive. While a particular diagnostic method may not provide a definitive diagnosis of a condition, it suffices if the method provides a positive indication that aids in diagnosis. “Prognostic” is the probability of development (such as severity) of a pathologic condition, such as COVID-19.

Diagnostic imaging: Coupling antibodies and their derivatives with positron emitting radionuclides for positron emission tomography (PET) is a process often referred to as immunoPET. While full length antibodies can make good immunoPET agents, their biological half-life necessitates waiting several days prior to imaging, resulting in an increase in non-target radiation doses. Smaller, single domain antibodies, such as camel or shark nanobodies, have biological half-lives amenable to same day imaging.

Drug: Any compound used to treat, ameliorate or prevent a disease or condition in a subject. In some aspects herein, the drug is an anti-viral agent, such as an anti-SARS-CoV-2 agent.

Effector molecule: The portion of a chimeric molecule that is intended to have a desired effect on a cell to which the chimeric molecule is targeted. Effector molecule is also known as an effector moiety (EM), therapeutic agent, diagnostic agent, or similar terms. Therapeutic agents (or drugs) include such compounds as nucleic acids, proteins, peptides, amino acids or derivatives, glycoproteins, radioisotopes, photon absorbers, lipids, carbohydrates, or recombinant viruses. Nucleic acid therapeutic and diagnostic moieties include antisense nucleic acids, derivatized oligonucleotides for covalent cross-linking with single or duplex DNA, and triplex forming oligonucleotides. Alternatively, the molecule linked to a targeting moiety, such as an anti-S2 protein antibody, may be an encapsulation system, such as a liposome or micelle that contains a therapeutic composition such as a drug, a nucleic acid (such as an antisense nucleic acid), or another therapeutic moiety that can be shielded from direct exposure to the circulatory system. Means of preparing liposomes attached to antibodies are known (see, for example, U.S. Pat. No. 4,957,735; and Connor et al., Pharm Ther 28:341-365, 1985). Diagnostic agents or moieties include radioisotopes and other detectable labels. Detectable labels useful for such purposes include radioactive isotopes such as ³⁵S, ¹¹C, ¹³N, ¹⁵O, ¹⁸F, ¹⁹F, ^{99m}Tc, ¹³¹I, ³H, ¹⁴C, ¹⁵N, ⁹⁰Y, ⁹⁹Tc, ¹¹¹In and ¹²⁵I, fluorophores, chemiluminescent agents, and enzymes.

Epitope: An antigenic determinant. These are particular chemical groups or peptide sequences on a molecule that are antigenic (that elicit a specific immune response). An antibody specifically binds a particular antigenic epitope on a polypeptide, such as the S2 subunit of the SARS-CoV-2 S protein.

Framework region: Amino acid sequences interposed between CDRs (and/or hypervariable regions). Framework regions of an immunoglobulin molecule include variable light and variable heavy framework regions.

Fusion protein: A protein comprising at least a portion of two different (heterologous) proteins. In some aspects, a fusion protein includes a single-domain monoclonal antibody fused to an Fc region.

Heterologous: Originating from a separate genetic source or species.

Host cell: Cells in which a vector can be propagated and its DNA expressed. The cell may be prokaryotic or eukaryotic. In some examples, the prokaryotic cell is an E. coli cell. In some examples, the eukaryotic cell is a human cell, such as a human embryonic kidney (HEK) cell. The term also includes any progeny of the subject host cell. It is understood that all progeny may not be identical to the parental cell since there may be mutations that occur during replication. However, such progeny are included when the term “host cell” is used.

Immobilized: Bound to a surface, such as a solid support. In one aspect, the solid surface is in the form of a bead, multiwell plate, paper, or nitrocellulose. The surface can include one or more polypeptides (e.g., single-domain monoclonal antibodies) provided herein.

Immune response: A response of a cell of the immune system, such as a B cell, T cell, or monocyte, to a stimulus. In one aspect, the response is specific for a particular antigen (an “antigen-specific response”). In one aspect, an immune response is a T cell response, such as a CD4⁺ response or a CD8⁺ response. In another aspect, the response is a B cell response, and results in the production of specific antibodies.

Immunoconjugate: A covalent linkage of an effector molecule to an antibody (such as a single-domain monoclonal antibody) or functional fragment thereof. The effector molecule can be, for example, a detectable label, a photon absorber (such as IR700), or a toxin (to form an immunotoxin, such as an immunotoxin comprising Pseudomonas exotoxin or a variant thereof). Specific, non-limiting examples of toxins include, but are not limited to, abrin, ricin, Pseudomonas exotoxin (PE, such as PE35, PE37, PE38, and PE40), diphtheria toxin (DT), botulinum toxin, or modified toxins thereof, or other toxic agents that directly or indirectly inhibit cell growth or kill cells. For example, PE and DT are highly toxic compounds that typically bring about death through liver toxicity. PE and DT, however, can be modified into a form for use as an immunotoxin by removing the native targeting component of the toxin (such as the domain Ia of PE and the B chain of DT) and replacing it with a different targeting moiety, such as an antibody. In one aspect, an antibody is joined to an effector molecule. In another aspect, an antibody joined to an effector molecule is further joined to a lipid or other molecule, such as to increase its half-life in the body. The linkage can be either by chemical or recombinant means. In one aspect, the linkage is chemical, wherein a reaction between the antibody moiety and the effector molecule has produced a covalent bond formed between the two molecules to form one molecule. A peptide linker (short peptide sequence) can optionally be included between the antibody and the effector molecule. Because immunoconjugates were originally prepared from two molecules with separate functionalities, such as an antibody and an effector molecule, they are also sometimes referred to as “chimeric molecules.” The term “chimeric molecule,” as used herein, therefore refers to a targeting moiety, such as a ligand or an antibody, conjugated (coupled) to an effector molecule. The term “conjugated” or “linked” refers to making two polypeptides into one contiguous polypeptide molecule.

Immunoliposome: A liposome with antigen-binding polypeptides (such as antibodies or antibody fragments) conjugated to its surface. Immunoliposomes can carry cytotoxic agents or other drugs to antibody-targeted cells, such as virus-infected cells.

Isolated: An “isolated” biological component, such as a nucleic acid, protein (including antibodies) or organelle, has been substantially separated or purified away from other biological components in the environment (such as a cell) in which the component naturally occurs, for example other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Label: A detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes. In one example, a “labeled antibody” refers to incorporation of another molecule in the antibody. For example, the label is a detectable marker, such as the incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Various methods of labeling polypeptides and glycoproteins are known and may be used. Examples of labels for polypeptides include, but are not limited to, the following: radioisotopes or radionucleotides (such as ³⁵S, ¹¹C, ¹³N, ¹⁵O, ¹⁸F, ¹⁹F, ^{99m}Tc, ¹³¹I, ³H, ¹⁴C, ¹⁵N, ⁹⁰Y ⁹⁹Tc, ¹¹In and ¹²⁵I), fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents, such as gadolinium chelates. In some aspects, labels are attached by spacer arms of various lengths to reduce potential steric hindrance.

Linker: In some cases, a linker is a peptide within an antibody binding fragment (such as an Fv fragment) which serves to indirectly bond the variable heavy chain to the variable light chain. “Linker” can also refer to a peptide serving to link a targeting moiety, such as an antibody, to an effector molecule, such as a cytotoxin or a detectable label. The terms “conjugating,” “joining,” “bonding” or “linking” refer to making two polypeptides into one contiguous polypeptide molecule, or to covalently attaching a radionuclide or other molecule to a polypeptide, such as an antibody. The linkage can be either by chemical or recombinant means. “Chemical means” refers to a reaction between the antibody moiety and the effector molecule such that there is a covalent bond formed between the two molecules to form one molecule.

Neutralizing antibody: An antibody that reduces the infectious titer of an infectious agent by binding to a specific antigen on the infectious agent, such as a virus (e.g., a coronavirus). In some aspects, an antibody that is specific for a coronavirus spike protein (such as the S2 subunit of the spike protein) neutralizes the infectious titer of SARS-CoV-2 (or variant thereof) and/or SARS-CoV. For example, an antibody that neutralizes SARS-CoV-2 may interfere with the virus by binding it directly and limiting entry into cells. Alternately, a neutralizing antibody may interfere with one or more post-attachment interactions of the pathogen with a receptor, for example, by interfering with viral entry and/or fusion with host cells. In some aspects, an antibody that specifically binds to SARS-CoV-2 and neutralizes SARS-CoV-2 inhibits infection of cells, for example, by at least 50%, by at least 60%, by at least 70%, by at least 80% or by at least 90%, compared to a control antibody. Similarly, an antibody can neutralize SARS-CoV by specifically binding to a SARS-CoV antigen (such as the spike protein) in such a way as to inhibit a biological function associated with SARS-CoV that inhibits infection.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carriers: The pharmaceutically acceptable carriers of use are conventional. Remington: The Science and Practice of Pharmacy, 22^{nd} ed., London, UK: Pharmaceutical Press, 2013,), describes compositions and formulations suitable for pharmaceutical delivery of the polypeptides, antibodies and other compositions disclosed herein. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Photoimmunotherapy: A targeted therapy that utilizes an antigen-specific antibody-photoabsorber conjugate that can be activated by near-infrared light to kill targeted cells. The photon absorber is typically based on phthalocyanine dye, such as a near infrared (NIR) phthalocyanine dye (for example, IRDye® 700DX, also know known as IR700). The antibody (for example, a S2-specific antibody) binds to the appropriate antigen (e.g., S protein) and the photo-activatable dye induces lethal damage to cell membranes after NIR-light exposure. NIR-light exposure (e.g., 690 nm) induces highly selective, necrotic cell death within minutes without damage to adjoining cells (see, for example, U.S. Application No. 2018/0236076). Thus, such methods can be used to kill cells infected with a coronavirus (e.g., SARS-CoV and/or SARS-CoV-2), such as using the antibodies provided herein.

Polypeptide: A polymer in which the monomers are amino acid residues joined together through amide bonds. When the amino acids are alpha-amino acids, either the L-optical isomer or the D-optical isomer can be used. The terms “polypeptide” and “protein” are used herein interchangeably and include standard amino acid sequences as well as modified sequences, such as glycoproteins. The term “polypeptide” is specifically intended to cover naturally occurring proteins, as well as proteins that are recombinantly or synthetically produced. In the context of the present disclosure, a “polypeptide” is any protein or polypeptide (natural, recombinant or synthetic) that is capable of specific binding to a target antigen, such as a coronavirus S protein or portion thereof, such as the S2 subunit. Thus, the polypeptides disclosed herein include at least one, such as one, two or three, CDR sequences that mediate specific binding to the target antigen. In some aspects, the polypeptide is a single-domain monoclonal antibody, such as a camel single-domain monoclonal antibody (V_{H}H) or a shark V_{NAR} antibody, isolated from a phage display library, or a modified form thereof (such as a humanized or chimeric single-domain monoclonal antibody). In other aspects, the polypeptide comprises fibronectin (adectin), albumin, protein A (affibody), a peptide aptamer, an affimer, an affitin, an anticalin, or another antibody mimetic (see, e.g., Yu et al., Annu Rev Anal Chem 10(1): 293-320, 2017; Ta and McNaughton, Future Med Chem 9(12): 1301-1304, 2017; Koutsoumpeli et al., Anal Chem 89(5): 3051-3058, 2017), or a similar protein in which one or more CDR sequences (and/or HV sequences) have been incorporated to confer specific binding to the target antigen.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop, such as a reduction in viral load. “Ameliorating” refers to the reduction in the number or severity of signs or symptoms of a disease, such as a coronavirus infection.

Purified: The term purified does not require absolute purity; rather, it is intended as a relative term. Thus, for example, a purified peptide preparation is one in which the peptide or protein is more enriched than the peptide or protein is in its environment within a cell. In one aspect, a preparation is purified such that the protein or peptide represents at least 50% of the total peptide or protein content of the preparation. Substantial purification denotes purification from other proteins or cellular components. A substantially purified protein is at least 60%, 70%, 80%, 90%, 95%, 98%, 99%, 99.9% or 99/99% pure. Thus, in one specific, non-limiting example, a substantially purified protein is 90% free of other proteins or cellular components.

Recombinant: A recombinant nucleic acid or protein is one that has a sequence that is not naturally occurring or has a sequence that is made by an artificial combination of two otherwise separated segments of sequence. This artificial combination is often accomplished by chemical synthesis or by the artificial manipulation of isolated segments of nucleic acids, for example, by genetic engineering techniques.

Sample (or biological sample): A biological specimen containing genomic DNA, RNA (including mRNA), protein, or combinations thereof, which can be obtained from a subject or the environment. Examples include, but are not limited to, sputum, saliva, mucus, nasal wash, peripheral blood, tissue, cells, urine, tissue biopsy, fine needle aspirate, surgical specimen, feces, cerebral spinal fluid (CSF), bronchoalveolar lavage (BAL) fluid, nasopharyngeal samples, oropharyngeal samples, and autopsy material. Environmental samples include those obtained from an environmental media, such as water, air, soil, dust, wood, as well as samples obtained by wiping or swabbing a surface.

SARS-CoV-2: A coronavirus of the genus betacoronavirus that first emerged in humans in 2019. This virus is also known as Wuhan coronavirus, 2019-nCoV, or 2019 novel coronavirus. Symptoms of SARS-CoV-2 infection include fever, chills, dry cough, shortness of breath, fatigue, muscle/body aches, headache, new loss of taste or smell, sore throat, nausea or vomiting, and diarrhea. Patients with severe disease can develop pneumonia, multi-organ failure, and death. The time from exposure to onset of symptoms is approximately 2 to 14 days. The SARS-CoV-2 virion includes a viral envelope with large spike glycoproteins. The SARS-CoV-2 genome, like most coronaviruses, has a common genome organization with the replicase gene included in the 5′-two thirds of the genome, and structural genes included in the 3′-third of the genome. The SARS-CoV-2 genome encodes the canonical set of structural protein genes in the order 5′-spike (S)-envelope (E)-membrane (M) and nucleocapsid (N)-3′. The term “SARS-CoV-2” includes variants thereof, such as, but not limited to, alpha (B.1.1.7 and Q lineages); beta (B.1.351 and descendent lineages); delta (B.1.617.2 and AY lineages); gamma (P.1 and descendent lineages); epsilon (B.1.427 and B.1.429); eta (B.1.525); iota (B.1.526); kappa (B.1.617.1); 1.617.3; mu (B.1.621, B.1.621.1); zeta (P.2); and omicron (B.1.1.529, BA.1, BA.1.1, BA.2, BA.3, BA.4 and BA.5 lineages)).

SARS Spike (S) protein: A class I fusion glycoprotein initially synthesized as a precursor protein of approximately 1256 amino acids for SARS-CoV, and 1273 amino acids for SARS-CoV-2. Individual precursor S polypeptides form a homotrimer and undergo glycosylation within the Golgi apparatus as well as processing to remove the signal peptide, and cleavage by a cellular protease between approximately position 679/680 for SARS-CoV, and 685/686 for SARS-CoV-2, to generate separate S1 and S2 polypeptide chains, which remain associated as S1/S2 protomers within the homotrimer, thereby forming a trimer of heterodimers. The S1 subunit is distal to the virus membrane and contains the receptor-binding domain (RBD) that is believed to mediate virus attachment to its host receptor. The S2 subunit is believed to contain the fusion protein machinery, such as the fusion peptide. S2 also includes two heptad-repeat sequences (HR1 and HR2) and a central helix typical of fusion glycoproteins, a transmembrane domain, and a cytosolic tail domain.

Sequence identity: The similarity between amino acid or nucleic acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide or nucleic acid molecule will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Homologs and variants of an antibody that specifically binds the S2 subunit of a coronavirus spike protein are typically characterized by possession of at least about 75%, for example at least about 80%, 90%, 95%, 96%, 97%, 98% or 99% sequence identity counted over the full-length alignment with the amino acid sequence of the antibody using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Small molecule: A molecule, typically with a molecular weight less than about 1000 Daltons, or in some aspects, less than about 500 Daltons, wherein the molecule is capable of modulating, to some measurable extent, an activity of a target molecule.

Subject: Living multi-cellular vertebrate organisms, a category that includes both human and non-human animals (such as veterinary subjects or wild animals), for example birds, pigs, mice, rats, rabbits, sheep, horses, cows, dogs, cats, ferrets, deer, otters, bank voles, raccoon dogs, tree shrews, fruit bats, hamsters, mink, and non-human primates (e.g., rhesus macaques, cynomolgus macaques, baboons, grivets and common marmosets). In some examples, the subject treated with the methods provided herein is infected with a coronavirus, such as SARS-CoV or SARS-CoV-2. In some examples, the subject treated with the methods provided herein has COVID-19. In some examples, the subject treated with the methods provided herein is infected with MERS-CoV, HKU1-CoV, OC43-CoV, 229E-CoV, or NL63-CoV.

Synthetic: Produced by artificial means in a laboratory, for example a synthetic nucleic acid or protein (for example, an antibody) can be chemically synthesized in a laboratory.

Therapeutically effective amount: The amount of agent, such as a polypeptide (e.g., a single-domain monoclonal antibody) that is alone (or in combination with other therapeutic agents) sufficient to prevent, treat (including prophylaxis), reduce and/or ameliorate the symptoms and/or underlying causes of a disease or disorder, for example to prevent, inhibit, and/or treat a coronavirus infection, such as a SARS-CoV and/or SARS-CoV-2 infection. In some aspects, a therapeutically effective amount is sufficient to reduce or eliminate a symptom of a disease, such as a SARS-CoV and/or SARS-CoV-2 infection. For instance, this can be the amount necessary to inhibit or prevent viral replication or to measurably alter outward symptoms of the viral infection, such as fever, cough, or difficulty breathing. In general, this amount will be sufficient to measurably inhibit virus replication or infectivity.

In one example, a desired response is to inhibit or reduce or prevent a SARS-CoV and/or SARS-CoV-2 infection. The SARS-CoV and/or SARS-CoV-2 infection does not need to be completely eliminated or reduced or prevented for the method to be effective. For example, administration of a therapeutically effective amount of the agent can decrease the SARS-CoV and/or SARS-CoV-2 infection (for example, as measured by infection of cells, or by number or percentage of subjects infected by SARS-CoV and/or SARS-CoV-2) by a desired amount, for example by at least 10%, at least 20%, at least 50%, at least 60%, at least 70%, at least 80%, at least 85%, at least 90%, at least 95%, at least 98%, at least 99% or even at least 100% (elimination or prevention of detectable coronavirus infection, as compared to a suitable control).

A therapeutically effective amount of an agent can be administered in a single dose, or in several doses, for example daily, during a course of treatment. However, the therapeutically effective amount can depend on the subject being treated, the severity and type of the condition being treated, and the manner of administration. A unit dosage form of the agent can be packaged in a therapeutic amount, or in multiples of the therapeutic amount, for example, in a vial (e.g., with a pierceable lid) or syringe having sterile components.

Toxin: A molecule that is cytotoxic for a cell. Toxins include abrin, ricin, Pseudomonas exotoxin (PE), diphtheria toxin (DT), botulinum toxin, saporin, restrictocin or gelonin, or modified toxins thereof. For example, PE and DT are highly toxic compounds that typically bring about death through liver toxicity. PE and DT, however, can be modified into a form for use as an immunotoxin by removing the native targeting component of the toxin (such as domain Ia of PE or the B chain of DT) and replacing it with a different targeting moiety, such as an antibody.

Vector: A nucleic acid molecule as introduced into a host cell, thereby producing a transformed host cell. A vector may include nucleic acid sequences that permit it to replicate in a host cell, such as an origin of replication. A vector may also include one or more selectable marker genes and other genetic elements known in the art. In some aspects, the vector is a virus vector, such as a lentivirus vector, an adenovirus vector, or an adeno-associated virus (AAV) vector.

### III. Polypeptides Specific for the S2 Subunit of Coronavirus Spike Protein

The vast majority of previously described SARS-CoV-2 neutralizing antibodies target the receptor binding domain (RBD) of the SARS-CoV-2 spike protein. Blocking the interaction of the RBD with ACE2 expressed on human cells is one mechanism for achieving virus neutralization. However, the S1 subunit is prone to mutation, resulting in the emergence of several SARS-CoV-2 variants. Mutations are less common the S2 subunit of the SARS-CoV-2 spike protein. The S2 subunit is responsible for virus-host membrane fusion, providing a second target for neutralization. It is hypothesized that neutralizing antibodies targeting this region of the spike protein will provide broader protection against SARS-CoV-2 variants, and that these S2-specific antibodies will be useful as pan-coronavirus neutralizing antibodies.

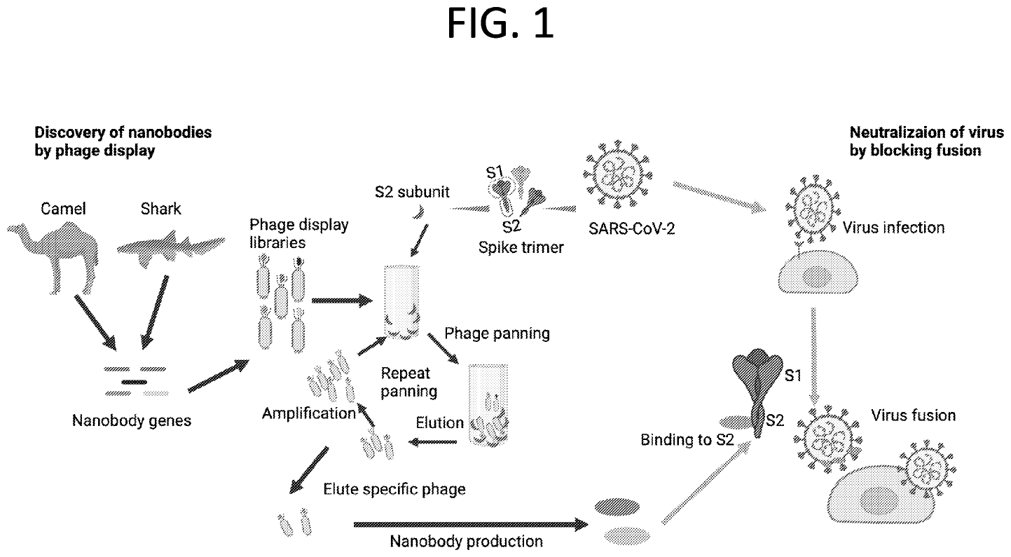

Naturally occurring single domain antibodies derived from camelid mammals or sharks are very small (approximately 15 kD) relative to mammalian immunoglobulins. Both camelid variable heavy homodimers (V_{H}H) and shark variable new antigen receptor (V_{NAR}) antibodies have unique conformations and can recognize protein cavities that are not accessible to conventional antibodies. The study disclosed herein screened nanobodies targeting S2 from both naïve adult nurse shark (Ginglymostoma cirratum) V_{NAR} and naïve dromedary camel (Camelus dromedaries) V_{H}H phage libraries. Both the SARS-CoV-2 S2 subunit and stabilized spike ectodomain trimer protein were used as baits to conduct phage panning for nanobody screening (FIG. 1).

Described herein is a panel of anti-S2 single-domain monoclonal antibodies (also called ‘nanobodies’) from shark V_{NAR} and camel V_{H}H single domain phage libraries. The disclosed antibodies can be used, for example, in the treatment or prevention of coronavirus infection, such as SARS-CoV-2 infection (such as treatment of COVID-19). The S2-specific antibodies can be used alone or in combination with other therapeutic agents, such as antibodies that target other spike protein epitopes, such as the RBD, as well as other anti-viral therapies. The present disclosure demonstrates that several nanobodies, including NCI-CoV-S2A9 (S2A9), can bind S2 subunits from other coronaviruses. S2A9 was identified as the most potent neutralizing antibody targeting the S2 subunit. S2A9 in the form of V_{NAR} (monovalent form) neutralized the original strain of SARS-CoV-2 pseudovirus at an IC₅₀ of 1385 nM. When the nanobodies were changed into a bivalent hFc fusion, their inhibitory activity significantly improved. The bivalent S2A9-hFc inhibited SARS-CoV-2 pseudovirus at an IC₅₀ of 80 nM. S2A9 V_{NAR} and S2A9-hFc also possess neutralizing activity against SARS-CoV-2 variants, including alpha, beta, gamma, delta, lambda, and omicron variants.

### Shark V_{NAR} Amino Acid Sequences

The amino acid sequences of the six shark nanobodies are provided below. Shark V_{NAR} are comprised of the following regions (N-terminal to C-terminal): FR1-CDR1-FR2-HV2-FR3a-HV4-FR3b-CDR3-FR4. CDRs (in bold) were determined using IMGT. HV2 and HV4 (underlined) were determined using annotation described in Stanfield et al. (Science 305:1770-1773, 2004) and Fennell et al. (J Mol Biol 400:155-170, 2010). Table 1 lists the amino acid positions of each CDR and HV.

NCI-CoV-S2A9
(SEQ ID NO: 1)
ARVDQTPRSVTKETGESLTINCVLRDASYALGRTCWYRKKSGSTNEESIS
KGGRYVETVNSGSKSFSLRINDLTVEDGGTYRCGVSTPWSCDYGPLCFYA
ACGDGTVVTVN
NCI-CoV-S2G8
(SEQ ID NO: 2)
ARVDQTPQTITKETGESLTINCVLRDSNCPLSNTYWYRKKSGSTNEESIS
KGGRYVETVNSGSKSFSLRINDLTVEDSGTYRCKVRTHWSICWEMGLEPN
YDVYGGGTVVTVN
NCI-CoV-S3A10
(SEQ ID NO: 3)
ARVDQTPRSVTKETGESLTINCVLRDASYALGSTCWYRKKSGSTNEESIS
KGGRYVETVNSGSKSFSLRINDLTVEDGGTYRCGRIQNTKAVLLPCVYAA
CGDGTVVTVN
NCI-CoV-S4A9
(SEQ ID NO: 4)
ARVDQTPRSVTKETGESLTINCVLRDASYALGSTCWYRKKSGSTNEESIS
KGGRYVETVNSGSKSFSLRINDLTVEDGGTYRCGVQSRWGAVPKLCSNYA
ACGDGTAVTVN
NCI-CoV-S4C12
(SEQ ID NO: 5)
ARVDQTPQTITKETGESLTINCVLRDSNCALSSTYWYRKKSGSTNEESIS
KGGRYVETVNSGSKSFSLRINDLTVEDSGTYRCKVGCWYGYESSLMNWSR
VYGGGTAVTVN
NCI-CoV-S6E1
(SEQ ID NO: 6)
ARVDQTPRSVTKETGESLTINCVLRDASYALGSTCWYRKKSGSTNEESIS
KGGRYVETVNSGSKSFSLRINDLTVEDSGTYRCKGYTPCMPDWVPDVYGM
GTVVTVN

All six of the shark VNAR antibodies include an HV2 having the amino acid sequence NEESISKG (SEQ ID NO: 25) and an HV4 having the amino acid sequence NSGSK (SEQ ID NO: 26).

TABLE 1
Positions of the CDRs and hypervariable regions of shark V_{NAR}

| | SEQ ID | | | | |
|---|---|---|---|---|---|
| VNAR | NO: | CDR1 | HV2 | HV4 | CDR3 |
| S2A9 | 1 | 26-33 | 45-52 | 60-64 | 86-101 |
| S2G8 | 2 | 26-33 | 45-52 | 60-64 | 86-103 |
| S3A10 | 3 | 26-33 | 45-53 | 60-64 | 86-100 |
| S4A9 | 4 | 26-33 | 45-52 | 60-64 | 86-101 |
| S4C12 | 5 | 26-33 | 45-52 | 60-64 | 86-101 |
| S6E1 | 6 | 26-33 | 45-52 | 60-64 | 86-97 |

### Camel V_{H}H Amino Acid Sequences

The amino acid sequences of the six camel nanobodies are provided below. Camel V_{H}H are comprised of the following regions (N-terminal to C-terminal): FR1-CDR1-FR2-CDR2-FR3-CDR3-FR4. Camel V_{H}H CDR residues were determined according to IMGT (italics), Kabat (underlined) and Paratome (bold). Tables 2-7 list the amino acid positions of CDR1, CDR2 and CDR3 for each antibody using IMGT, Kabat and Paratome.

NCI-CoV-CLA2
(SEQ ID NO: 7)
QVQLVESGGGSVQPGGSLRLSCAVSGYTSSSYCFGWFRQAPGKDREGIIA
MEPGNTYTAGAVKGRFTISRDNAKNMVYLQMNSLKPDDTAMYYCAASPYP
GTWLNPDLFDTWGQGTQVTVSS

TABLE 2
Positions of the CDRs in Camel V_{H}H CLA2 (SEQ ID NO: 7)

| CLA2 | CDR1 | CDR2 | CDR3 |
|---|---|---|---|
| IMGT | 26-32 | 50-55 | 95-111 |
| Kabat | 26-32 | 50-55 | 97-111 |
| Paratome | 27-35 | 47-58 | 96-111 |

NCI-CoV-CG2G2
(SEQ ID NO: 8)
AVQLVESGGGSVPPGGSLTLSCTVSDNIFRTYAVGWFRQAPEKRCQWIAT
LNADESTEYNVAVTGRFTISRDNSGRSVYLQMDNLNSEDTAIYYCASLAR
KWSCHDSWGRGTQVTVSS

TABLE 3
Positions of the CDRs in Camel V_{H}H CG2G2 (SEQ ID NO: 8)

| CG2G2 | CDR1 | CDR2 | CDR3 |
|---|---|---|---|
| IMGT | 26-33 | 51-58 | 96-107 |
| Kabat | 31-35 | 50-65 | 98-107 |
| Paratome | 26-35 | 44-61 | 97-107 |

NCI-CoV-CGH3
(SEQ ID NO: 9)
AVQLVESGGGLVQPGGSLRLSCAASGFTFSSYAMGWVRQAPGKGLEWISA
INSGGDIRYYADSVKGRFTISRDNAKNTLYLQMNSLKPEDTAVYYCATDD
EILSYGLGTSFDYWGQGTQVTVSS

TABLE 4
Positions of the CDRs in Camel V_{H}H CGH3 (SEQ ID NO: 9)

| CGH3 | CDR1 | CDR2 | CDR3 |
|---|---|---|---|
| IMGT | 26-33 | 51-58 | 97-113 |
| Kabat | 31-35 | 50-66 | 99-113 |
| Paratome | 27-35 | 47-60 | 98-113 |

NCI-CoV-CGF10
(SEQ ID NO: 10)
AVQLVESGGGLVQPGGSLRLSCAASGFTFSSYGMHWVRQAPGKGLERVAG
INWDGSNSYYTETVKGRFTISRDNAKNTAYLQMNSLKPEDTAMYYCRTLI
LDSDCDPGSTYRYAWGQGTQVTVSS

TABLE 5
Positions of the CDRs in Camel V_{H}H CGF10 (SEQ ID NO: 10)

| CGF10 | CDR1 | CDR2 | CDR3 |
|---|---|---|---|
| IMGT | 26-33 | 51-58 | 97-113 |
| Kabat | 31-35 | 50-66 | 99-114 |
| Paratome | 27-35 | 47-61 | 97-114 |

NCI-CoV-CG2D11
(SEQ ID NO: 11)
AVQLVDSGGGSVQAGGSLRLSCAGSAFTYSSYCMGWFRQAPGKEREAVAA
INSGTSSTYYADSVKGRFTVSQDNANNTVYLLMISLKPEDTAIYICAALV
APDYDDYDVAAPDNFDYWGQGTQVTVSS

TABLE 6
Positions of the CDRs in Camel V_{H}H CG2D11 (SEQ ID NO: 11)

| CG2D11 | CDR1 | CDR2 | CDR3 |
|---|---|---|---|
| IMGT | 26-33 | 51-58 | 97-117 |
| Kabat | 31-35 | 50-66 | 99-117 |
| Paratome | 26-35 | 47-60 | 98-117 |

NCI-CoV-CL3H4
(SEQ ID NO: 12)
DVQLVESGGGAVKPGGSLTLTCTVQEGSRNCVGWFRQGSGKDRQAVAMIN
GNGGTTNYDDRVKGRFQISRDGSLVHLRMNDVKLGDTDIYYCRQGQTYDC
WGGTWTDSGWGQGTQVTVSS

TABLE 7
Positions of the CDRs in Camel V_{H}H CL3H4 (SEQ ID NO: 12)

| CL3H4 | CDR1 | CDR2 | CDR3 |
|---|---|---|---|
| IMGT | 26-31 | 49-56 | 93-109 |
| Kabat | 31-33 | 48-64 | 95-109 |
| Paratome | 26-31 | 42-59 | 93-109 |

Provided herein are polypeptides that bind (for example, specifically bind) the S2 subunit of the coronavirus spike protein, such as SARS-CoV-2 (or variants thereof), SARS-CoV, MERS-CoV and/or HCoV-OC43 spike protein (such as the S2 subunit of the spike protein). In some aspects, the polypeptide is a monoclonal antibody, for example a single-domain monoclonal antibody, such as a shark V_{NAR} single-domain antibody or a camel V_{H}H single-domain antibody. Also provided are compositions that include one or more of such antibodies, for example a composition that includes a pharmaceutically acceptable carrier (such as water or saline).

In some aspects, the polypeptide (for example, single-domain monoclonal antibody) includes at least a portion of the amino acid sequence set forth herein as any one of SEQ ID NOs: 1-12, such as one or two CDR sequences from any one of antibodies S2A9, S2G8, S3A10, S4A9, S4C12 and 6E1 (SEQ ID NOs: 1-6, respectively), or one or more (such as one, two or three) CDR sequences from any one of antibodies CLA2, CG2G2, CGH3, CGF10, CG2D11 and CL3H4, as determined using any CDR numbering scheme (such as IMGT, Kabat, Paratome or Chothia, or any combination thereof; or using the annotation described in Stanfield et al. 2004 and/or Fennell et al. 2010 for shark V_{NAR}). In some examples, the polypeptide includes the CDR1 and CDR3 sequences of any one of SEQ ID NOs: 1-6. In particular examples, the CDR sequences are determined using IMGT. In specific non-limiting examples, the polypeptide further includes the HV2 sequence of SEQ ID NO: 25 and/or the HV4 sequence of SEQ ID NO: 26. In other examples, the polypeptide includes the CDR1, CDR2 and CDR3 sequences of any one of SEQ ID NOs: 7-12. In particular examples, the CDR sequences are determined using the Kabat, IMGT or Paratome numbering schemes, or a combination of the Kabat, IMGT and Paratome numbering schemes.

In some aspects, the polypeptide includes the CDR1 and CDR3 sequences of NCI-CoV-S2A9 (SEQ ID NO: 1). In some examples, the CDR1 and CDR3 sequences respectively include residues 26-33 and 86-101 of SEQ ID NO: 1. In some examples, the polypeptide further includes a HV2 region comprising the amino acid sequence of SEQ ID NO: 25 and/or a HV4 region comprising the amino acid sequence of SEQ ID NO: 26. In particular examples, the amino acid sequence of the polypeptide is at least 80%, at least 85%, at least 90%, at least 95%, at least 96%, at least 97%, at least 98% or at least 99% identical to SEQ ID NO: 1. In specific examples, the amino acid sequence of the polypeptide comprises or consists of SEQ ID NO: 1.

In some aspects, the polypeptide includes the CDR1 and CDR3 sequences of NCI-CoV-S2G8 (SEQ ID NO: 2). In some examples, the CDR1 and CDR3 sequences respectively include residues 26-33 and 86-103 of SEQ ID NO: 2. In some examples, the polypeptide further includes a HV2 region comprising the amino acid sequence of SEQ ID NO: 25 and/or a HV4 region comprising the amino acid sequence of SEQ ID NO: 26. In particular examples, the amino acid sequence of the polypeptide is at least 80%, at least 85%, at least 90%, at least 95%, at least 96%, at least 97%, at least 98% or at least 99% identical to SEQ ID NO: 2. In specific examples, the amino acid sequence of the polypeptide comprises or consists of SEQ ID NO: 2.

In some aspects, the polypeptide includes the CDR1 and CDR3 sequences of NCI-CoV-S3A10 (SEQ ID NO: 3). In some examples, the CDR1 and CDR3 sequences respectively include residues 26-33 and 86-100 of SEQ ID NO: 3. In some examples, the polypeptide further includes a HV2 region comprising the amino acid sequence of SEQ ID NO: 25 and/or a HV4 region comprising the amino acid sequence of SEQ ID NO: 26. In particular examples, the amino acid sequence of the polypeptide is at least 80%, at least 85%, at least 90%, at least 95%, at least 96%, at least 97%, at least 98% or at least 99% identical to SEQ ID NO: 3. In specific examples, the amino acid sequence of the polypeptide comprises or consists of SEQ ID NO: 3.

In some aspects, the polypeptide includes the CDR1 and CDR3 sequences of NCI-CoV-S4A9 (SEQ ID NO: 4). In some examples, the CDR1 and CDR3 sequences respectively include residues 26-33 and 86-101 of SEQ ID NO: 4. In some examples, the polypeptide further includes a HV2 region comprising the amino acid sequence of SEQ ID NO: 25 and/or a HV4 region comprising the amino acid sequence of SEQ ID NO: 26. In particular examples, the amino acid sequence of the polypeptide is at least 80%, at least 85%, at least 90%, at least 95%, at least 96%, at least 97%, at least 98% or at least 99% identical to SEQ ID NO: 4. In specific examples, the amino acid sequence of the polypeptide comprises or consists of SEQ ID NO: 4.

In some aspects, the polypeptide includes the CDR1 and CDR3 sequences of NCI-CoV-S4C12 (SEQ ID NO: 5). In some examples, the CDR1 and CDR3 sequences respectively include residues 26-33 and 86-101 of SEQ ID NO: 5. In some examples, the polypeptide further includes a HV2 region comprising the amino acid sequence of SEQ ID NO: 25 and/or a HV4 region comprising the amino acid sequence of SEQ ID NO: 26. In particular examples, the amino acid sequence of the polypeptide is at least 80%, at least 85%, at least 90%, at least 95%, at least 96%, at least 97%, at least 98% or at least 99% identical to SEQ ID NO: 5. In specific examples, the amino acid sequence of the polypeptide comprises or consists of SEQ ID NO: 5.

In some aspects, the polypeptide includes the CDR1 and CDR3 sequences of NCI-CoV-6E1 (SEQ ID NO: 6). In some examples, the CDR1 and CDR3 sequences respectively include residues 26-33 and 86-97 of SEQ ID NO: 6. In some examples, the polypeptide further includes a HV2 region comprising the amino acid sequence of SEQ ID NO: 25 and/or a HV4 region comprising the amino acid sequence of SEQ ID NO: 26. In particular examples, the amino acid sequence of the polypeptide is at least 80%, at least 85%, at least 90%, at least 95%, at least 96%, at least 97%, at least 98% or at least 99% identical to SEQ ID NO: 6. In specific examples, the amino acid sequence of the polypeptide comprises or consists of SEQ ID NO: 6.

In some aspects, the polypeptide includes the CDR1, CDR2 and CD3 sequences of NCI-CoV-CLA2 (SEQ ID NO: 7). In some examples, the CDR1, CDR2 and CDR3 sequences respectively include residues 26-32, 50-55 and 95-111 of SEQ ID NO: 7; residues 26-32, 50-55 and 97-111 of SEQ ID NO: 7; or residues 27-35, 47-58 and 96-111 of SEQ ID NO: 7. In particular examples, the amino acid sequence of the polypeptide is at least 80%, at least 85%, at least 90%, at least 95%, at least 96%, at least 97%, at least 98% or at least 99% identical to SEQ ID NO: 7. In specific examples, the amino acid sequence of the polypeptide comprises or consists of SEQ ID NO: 7.

In some aspects, the polypeptide includes the CDR1, CDR2 and CD3 sequences of NCI-CoV-CG2G2 (SEQ ID NO: 8). In some examples, the CDR1, CDR2 and CDR3 sequences respectively includes residues 26-33, 51-58 and 96-107 of SEQ ID NO: 8; residues 31-35, 50-65 and 98-107 of SEQ ID NO: 8; or residues 26-35, 44-61 and 97-107 of SEQ ID NO: 8. In particular examples, the amino acid sequence of the polypeptide is at least 80%, at least 85%, at least 90%, at least 95%, at least 96%, at least 97%, at least 98% or at least 99% identical to SEQ ID NO: 8. In specific examples, the amino acid sequence of the polypeptide comprises or consists of SEQ ID NO: 8.

In some aspects, the polypeptide includes the CDR1, CDR2 and CDR3 sequences of NCI-CoV-CGH3 (SEQ ID NO: 9). In some examples, the CDR1, CDR2 and CDR3 sequences respectively include residues 26-33, 51-58 and 97-113 of SEQ ID NO: 9; residues 31-35, 50-66 and 99-113 of SEQ ID NO: 9; or residues 27-35, 47-60 and 98-113 of SEQ ID NO: 9. In particular examples, the amino acid sequence of the polypeptide is at least 80%, at least 85%, at least 90%, at least 95%, at least 96%, at least 97%, at least 98% or at least 99% identical to SEQ ID NO: 9. In specific examples, the amino acid sequence of the polypeptide comprises or consists of SEQ ID NO: 9.

In some aspects, the polypeptide includes the CDR1, CDR2 and CDR3 sequences of NCI-CoV-CGF10 (SEQ ID NO: 10). In some examples, the CDR1, CDR2 and CDR3 sequences respectively include residues 26-33, 51-58 and 97-113 of SEQ ID NO: 10; residues 31-35, 50-66 and 99-114 of SEQ ID NO: 10; or residues 27-35, 47-61 and 97-114 of SEQ ID NO: 10. In particular examples, the amino acid sequence of the polypeptide is at least 80%, at least 85%, at least 90%, at least 95%, at least 96%, at least 97%, at least 98% or at least 99% identical to SEQ ID NO: 10. In specific examples, the amino acid sequence of the polypeptide comprises or consists of SEQ ID NO: 10.

In some aspects, the polypeptide includes the CDR1, CDR2 and CDR3 sequences of NCI-CoV-CG2D11 (SEQ ID NO: 11). In some examples, the CDR1, CDR2 and CDR3 sequences respectively include residues 26-33, 51-58 and 97-117 of SEQ ID NO: 11; residues 31-35, 50-66 and 99-117 of SEQ ID NO: 11; or residues 26-35, 47-60 and 98-117 of SEQ ID NO: 11. In particular examples, the amino acid sequence of the polypeptide is at least 80%, at least 85%, at least 90%, at least 95%, at least 96%, at least 97%, at least 98% or at least 99% identical to SEQ ID NO: 11. In specific examples, the amino acid sequence of the polypeptide comprises or consists of SEQ ID NO: 11.

In some aspects, the polypeptide includes the CDR1, CDR2 and CDR3 sequences of NCI-CoV-CL3H4 (SEQ ID NO: 12). In some examples, the CDR1, CDR2 and CDR3 sequences respectively include residues 26-31, 49-56 and 93-109 of SEQ ID NO: 12; residues 31-33, 48-64 and 95-109 of SEQ ID NO: 12; or residues 26-31, 42-59 and 93-109 of SEQ ID NO: 12. In particular examples, the amino acid sequence of the polypeptide is at least 80%, at least 85%, at least 90%, at least 95%, at least 96%, at least 97%, at least 98% or at least 99% identical to SEQ ID NO: 12. In specific examples, the amino acid sequence of the polypeptide comprises or consists of SEQ ID NO: 12.

In some aspects, the polypeptide is a single-domain monoclonal antibody. In some examples, the single-domain monoclonal antibody is a camel V_{H}H single-domain antibody. In other examples, the single-domain monoclonal antibody is a shark V_{NAR} single-domain antibody. In some examples, the single-domain monoclonal antibody is a humanized single-domain monoclonal antibody or a chimeric single-domain monoclonal antibody. In other examples, the polypeptide is a recombinant fibronectin or albumin.

Further provided herein are polypeptide (for example, antibody) compositions that include at least two, at least three, at least four or at least five different polypeptides specific for the S2 subunit as disclosed herein. The polypeptides can each bind a separate epitope of the S2 subunit or can bind overlapping epitopes. Also provided are antibody compositions that include at least one polypeptide disclosed herein and at least one antibody that specifically binds a different coronavirus antigen or a different epitope of the spike protein, such as the S1 subunit and/or RBD.

In some examples, the polypeptide compositions further include a pharmaceutically acceptable carrier, such as water or saline. In some examples, the polypeptide composition is formulated for administration by inhalation. In other examples, the polypeptide is formulated for oral administration. In specific examples, the composition for oral administration includes bacteria or yeast engineered to express a disclosed polypeptide (such as a single-domain monoclonal antibody).

In some examples, the polypeptide composition is lyophilized.

Also provided are fusion proteins that include an S2 subunit-specific polypeptide (for example, antibody) disclosed herein and a heterologous protein. In some aspects, the heterologous protein is an Fc protein or a leucine zipper. A single-domain antibody can be fused to an Fc region to generate a bivalent antibody (V_{H}H-Fc or V_{N}AR-Fc). In some examples, the Fc protein is a human Fc protein, such as the human IgG1 Fc. In particular non-limiting examples, the fusion protein comprises a single-domain antibody disclosed herein, a hinge region and an Fc domain (such as the human IgG1 Fc domain). In one specific example, the fusion protein further includes a linker, such as an Ala-Ala-Ala linker located between the single-domain monoclonal antibody and the hinge region. In some aspects, provided is a disclosed single-domain monoclonal antibody in an IgG, IgA or IgM format.

Also provided herein are chimeric antigen receptors (CARs) that include a polypeptide (such as a single-domain monoclonal antibody) disclosed herein. In some aspects, the CAR further includes a hinge region, a transmembrane domain, a costimulatory signaling moiety, a signaling domain, or any combination thereof. In specific non-limiting examples, the hinge region comprises a CD8a hinge region, the transmembrane domain comprises a CD8a transmembrane domain, the costimulatory signaling moiety comprises a 4-1BB signaling moiety and/or the signaling domain comprises a CD3ζ signaling domain.

Also provided herein are S2 subunit-specific polypeptides (for example, antibodies) modified to enable their use with a universal CAR system. In some aspects, the S2 subunit-specific polypeptide is fused to one component of a specific binding pair. In some examples, the polypeptide is fused to a leucine zipper or biotin.

Further provided are cells expressing an S2 subunit-specific CAR. In some examples, the cell is a T lymphocyte, such as a CTL, a B cell, a natural killer (NK) cell, a macrophage or an induced pluripotent stem cell. In some examples, the cells are allogeneic cells, such as allogeneic cells obtained from a healthy donor. In specific non-limiting examples, the cells, such as T cells, are genetically modified to express the CAR and optionally to disrupt expression of the endogenous TCR. CARs and CAR-expressing cells are further described in section IV.

Also provided herein are immunoconjugates that include a polypeptide (for example, single-domain antibody) disclosed herein and an effector molecule. In some aspects, the effector molecule is a toxin, such as, but not limited to, Pseudomonas exotoxin or a variant thereof, such as PE38. In other aspects, the effector molecule is a detectable label, such as, but not limited to, a fluorophore, an enzyme or a radioisotope. In other aspects, the effector molecule is a photon absorber, such as IR700. Immunoconjugates comprising a photon absorber can be used for photoimmunotherapy or in vivo diagnostic imaging. Immunoconjugates are further described in section V.

Further provided herein are antibody-drug conjugates (ADCs) that include a drug conjugated to a polypeptide (for example, single-domain antibody) disclosed herein. In some aspects, the drug is a small molecule, for example an anti-viral agent, anti-microtubule agent, an anti-mitotic agent and/or a cytotoxic agent. In some examples, the anti-viral agent is ribavirin, remdesivir, galidesivir, arbidol, favipiravir, baricitinib, or lopinavir/ritonavir. ADCs are further described in section VI.

Also provided herein are multi-specific antibodies that include a polypeptide (for example, single-domain antibody) disclosed herein and at least one additional monoclonal antibody or antigen-binding fragment thereof. In some aspects, the multi-specific antibody is a bispecific antibody. In other aspects, the multi-specific antibody is a trispecific antibody. Multi-specific antibodies are further described in section VII.

Further provided herein are antibody-nanoparticle conjugates that include a nanoparticle conjugated to a polypeptide (for example, single-domain antibody) disclosed herein. In some aspects, the nanoparticle comprises a polymeric nanoparticle, nanosphere, nanocapsule, liposome, dendrimer, polymeric micelle, or niosome. In some aspects, the nanoparticle includes a cytotoxic agent or an anti-viral agent. In some examples, the anti-viral agent is ribavirin, remdesivir, galidesivir, arbidol, favipiravir, baricitinib, molnupiravir, or lopinavir/ritonavir. Antibody-nanoparticle conjugates are further described in section VIII.

Further provided herein are nucleic acid molecules that encode a polypeptide, an antibody, fusion protein, single-domain monoclonal antibody in an IgG, IgA or IgM format, CAR, immunoconjugate, or multiple-specific antibody disclosed herein. In some aspects, the nucleic acid molecule is operably linked to a promoter, such as an inducible or constitutive promoter. Vectors that include the disclosed nucleic acid molecules are also provided. In some examples, the vector is an expression vector. In other examples, the vector is a viral vector. Isolated cells that include a nucleic acid molecule are vector disclosed herein are further provided. In some examples, the isolated cell is a prokaryotic cell, such as an E. coli cell. In other examples, the isolated cell is a mammalian cell, such as a human cell. Nucleic acid molecules are further described in section IX.

Compositions that include a pharmaceutically acceptable carrier and a polypeptide (for example, single-domain monoclonal antibody), fusion protein (such as an Fc fusion, or nanobody in an IgG, IgA or IgM format), CAR, isolated cell (such as a CAR expressing cell, for example a CAR T cell, a CAR B cell, a CAR NK cell, a CAR macrophage, or a CAR iPSC), immunoconjugate, ADC, multi-specific antibody, antibody-nanoparticle conjugate, isolated nucleic acid molecule or vector disclosed herein are further provided by the present disclosure. In some examples, the composition includes a single-domain monoclonal antibody-Fc fusion protein (which forms a bivalent antibody). Compositions are further described in section X.

Further provided are methods of treating a coronavirus infection in a subject, such as a subject infected with SARS-CoV-2 or SARS-CoV, and/or a subject with COVID-19 disease. In some aspects, the method includes administering to the subject a therapeutically effective amount of a polypeptide (for example, single-domain monoclonal antibody), fusion protein (such as a V_{H}H-Fc or V_{N}AR-Fc), CAR, isolated cell (such as a CAR expressing cell, for example a CAR T cell, a CAR B cell, a CAR NK cell or a CAR macrophage), immunoconjugate, ADC, multi-specific antibody, antibody-nanoparticle conjugate, isolated nucleic acid molecule or vector disclosed herein, thereby treating the coronavirus infection. In some aspects, administration is by inhalation, such as with an inhaler or nebulizer. In some aspects, administration is by injection, such as intravenous injection. In some examples, the coronavirus is SARS-CoV, SARS-CoV-2 (or variant thereof), MERS-CoV, HKU1-CoV, OC43-CoV, 229E-CoV, or NL63-CoV. In specific examples, the coronavirus is SARS-CoV-2 or SARS-CoV. Such polypeptides (for example, monoclonal antibodies) can be used alone or in combination with other therapeutic agents. In some aspects, the subject is a human subject. In other aspects, the subject is a non-human animal subject, such as a non-human primate, cat, dog, bank vole, ferret, fruit bat, hamster, mink, otter, pig, rabbit, raccoon dog, tree shrew or deer. Therapeutic methods are further described in section XI.

Also provided are methods of detecting a coronavirus, or a coronavirus S protein, in a sample, such as one obtained from a subject or the environment. In some aspects, the method includes contacting the sample with a polypeptide (for example, antibody) disclosed herein and detecting binding of the polypeptide to the sample. Further provided are methods of detecting coronavirus in the environment. Further provided are methods of diagnosing a subject as having a coronavirus infection. In some aspects, the method includes contacting a sample obtained from the subject with a polypeptide disclosed herein and detecting binding of the polypeptide to the sample, thereby diagnosing the subject as having a coronavirus infection. In some examples of these methods, the polypeptide is directly labeled. In other examples, the method includes contacting the polypeptide with a detection antibody, and detecting the binding of the detection antibody to the polypeptide, thereby detecting the coronavirus in the sample or diagnosing the subject as having a coronavirus infection. In some examples, the sample is obtained from a subject suspected of having a coronavirus infection. In some examples, the coronavirus is SARS-CoV, SARS-CoV-2, MERS-CoV, HKU1-CoV, OC43-CoV, 229E-CoV, or NL63-CoV. In specific examples, the coronavirus is SARS-CoV-2 or SARS-CoV. In some aspects, the subject is a human subject. In other aspects, the subject is a non-human animal subject, such as a non-human primate, cat, dog, bank vole, ferret, fruit bat, hamster, mink, otter, pig, rabbit, raccoon dog, tree shrew or deer. Diagnostic and detection methods are further described in section XII.

Further provided herein are solid supports that include one or more of the S2 subunit-specific polypeptides disclosed herein. In some aspects, the solid support comprises a bead, microchip, multiwell plate, or nitrocellulose having attached thereto one or more of the disclosed polypeptides (such as single-domain antibodies). Further provided is a method of detecting a coronavirus in a sample that includes contacting the sample with the solid support having attached thereto one or more of the disclosed antibodies and detecting binding of the coronavirus to the one or more antibodies attached to the solid support, thereby detecting coronavirus in the sample. In some examples, the coronavirus is SARS-CoV, SARS-CoV-2 (or variant thereof), MERS-CoV, HKU1-CoV, OC43-CoV, 229E-CoV, or NL63-CoV. In specific examples, the coronavirus is SARS-CoV-2 or SARS-CoV. In some aspects, the sample is an environmental sample or a biological sample obtained from a subject. In some examples, the environmental sample is a water, air, or soil sample, or a sample from a swabbed surface.

### IV. Chimeric Antigen Receptors (CARs)

The disclosed nanobodies can be used to produce CARs and/or immune cells (such as T cells, B cells, natural killer (NK) cells, or macrophages) or induced pluripotent stem cells (iPSCs) engineered to express CARs. In some aspects, CARs include a binding moiety, an extracellular hinge and spacer element, a transmembrane region and an endodomain that performs signaling functions (Cartellieri et al., J Biomed Biotechnol 2010:956304, 2010; Dai et al., J Natl Cancer Inst 108(7):djv439, 2016). In some instances, the binding moiety is an antigen binding fragment of a monoclonal antibody, such as a scFv, or a single-domain antibody (such as a camel V_{H}H or shark V_{NAR}). The spacer/hinge region typically includes sequences from IgG subclasses, such as IgG1, IgG4, IgD, CD8, or CD28 domains. The transmembrane domain can be derived from a variety of different T cell proteins, such as CD3ζ, CD4, CD8, CD28 or inducible T cell co-stimulator (ICOS). Several different endodomains have been used to generate CARs. For example, the endodomain can consist of a signaling chain having an ITAM, such as CD3ζ or FcERIγ. In some instances, the endodomain further includes the intracellular portion of at least one additional co-stimulatory domain, such as CD28, 4-1BB (CD137, TNFRSF9), OX-40 (CD134), ICOS, CD27, MYD88-CD40, killer cell immunoglobulin-like receptor 2DS2 (KIR2DS2) and/or DAP10.

In some aspects, the CAR is a two-chained antibody-T cell receptor (AbTCR), which includes the transmembrane and intracellular domains of the γ and δ chains of the TCR (see, e.g., Xu et al., Cell Discovery 4:62, 2018), wherein each of the γ and δ chains is fused to a spike protein-specific single-domain antibody disclosed herein. When expressed in T cells, the AbTCR engages endogenous CD3 complexes to induce T cell activation (Xu et al., Cell Discovery 4:62, 2018).

In some aspects, the CAR is a synthetic T cell receptor and antigen receptor (STAR), which is a double-chain chimeric receptor that includes an antigen-binding domain (such a spike protein-specific monoclonal antibody) and the constant regions of the TCR that engage endogenous CD3 complexes (Liu et al., Sci Transl Med 13(586):eabb5191, 2021).

Immune cells (e.g., T cells, B cells, NK cells or macrophages) or iPSCs expressing CARs can be used to target a specific cell type, such as a coronavirus-infected cell. Thus, the nanobodies disclosed herein can be used to engineer immune cells or iPSCs that express a CAR containing the S2 subunit-specific monoclonal antibody, thereby targeting the engineered immune cells or iPSCs to cells infected with coronavirus (such as SARS-CoV-2 or SARS-CoV) and thereby expressing coronavirus spike protein.

Multispecific (such as bispecific) or bicistronic CARs are also contemplated by the present disclosure. In some aspects, the multispecific or bispecific CAR includes a nanobody specific for the S2 subunit of SARS-CoV-2 spike protein and a monoclonal antibody specific for a different antigen, or a different epitope of the spike protein, such as the S1 subunit. Similarly, a bicistronic CAR includes two CAR molecules expressed from the same construct where one CAR molecule is an S2 subunit-targeted CAR and the second CAR targets a second antigen, or a different epitope of the spike protein, such as the S1 subunit. See, for example, Qin et al., Blood 130:810, 2017; and WO/2018/213337.

Accordingly, provided herein are CARs that include an S2 subunit-specific antibody, such as any one of the nanobodies disclosed herein. Also provided are isolated nucleic acid molecules and vectors encoding the CARs (including bispecific and bicistronic CARs), and host cells, such as immune cells (e.g., T cells, B cells, NK cells, or macrophages) or induced pluripotent stem cells (iPSCs) expressing the CARs, bispecific CAR or bicistronic CARs. Immune cells or iPSCs expressing CARs comprised of an S2 subunit-specific monoclonal antibody can be used for the treatment of a coronavirus infection (such as a SARS-CoV-2 or SARS-CoV infection). In some aspects herein, the CAR is a bispecific CAR. In other aspects herein, the CAR is a bicistronic CAR.

In some aspects, the CAR includes a signal peptide sequence, for example, N-terminal to the antigen binding domain. The signal peptide sequence can be any suitable signal peptide sequence, such as a signal sequence from granulocyte-macrophage colony-stimulating factor receptor (GMCSFR), immunoglobulin light chain kappa, or IL-2. While the signal peptide sequence may facilitate expression of the CAR on the surface of the cell, the presence of the signal peptide sequence in an expressed CAR is not necessary in order for the CAR to function. Upon expression of the CAR on the cell surface, the signal peptide sequence may be cleaved off of the CAR. Accordingly, in some aspects, the CAR lacks a signal peptide sequence.

In some aspects, the CARs disclosed herein are expressed from a construct (such as from a lentivirus or other viral vector) that also expresses a truncated version of human EGFR (huEGFRt). The CAR and huEGFRt are separated by a self-cleaving peptide sequence (such as T2A) such that upon expression in a transduced cell, the CAR is cleaved from huEGFRt (see, e.g., WO 2019/094482, which is herein incorporated by reference).

The human epidermal growth factor receptor is comprised of four extracellular domains, a transmembrane domain and three intracellular domains. The EGFR domains are found in the following N-terminal to C-terminal order: Domain I-Domain II-Domain III-Domain IV-transmembrane (TM) domain-juxtamembrane domain-tyrosine kinase domain-C-terminal tail. Domain I and Domain III are leucine-rich domains that participate in ligand binding. Domain II and Domain IV are cysteine-rich domains and do not make contact with EGFR ligands. Domain II mediates formation of homo- or hetero-dimers with analogous domains from other EGFR family members, and Domain IV can form disulfide bonds with Domain II. The EGFR TM domain makes a single pass through the cell membrane and may play a role in protein dimerization. The intracellular domain includes the juxtamembrane domain, tyrosine kinase domain and C-terminal tail, which mediate EGFR signal transduction (Wee and Wang, Cancers 9(52), doi:10.3390/cancers9050052; Ferguson, Annu Rev Biophys 37:353-373, 2008; Wang et al., Blood 118(5):1255-1263, 2011).

A truncated version of human EGFR, referred to as “huEGFRt” includes only Domain III, Domain IV and the TM domain. Thus, huEGFRt lacks Domain I, Domain II, and all three intracellular domains. huEGFRt is not capable of binding EGF and lacks signaling activity. However, this molecule retains the capacity to bind particular EGFR-specific monoclonal antibodies, such as FDA-approved cetuximab (PCT Publication No. WO 2011/056894).

Transduction of immune cells (such as T cells, B cells, NK cells or macrophages) or iPSCs with a construct (such as a lentivirus vector) encoding both huEGFRt and an S2 subunit-specific CAR disclosed herein allows for selection of transduced cells using labelled EGFR monoclonal antibody cetuximab (ERBITUX™). For example, cetuximab can be labeled with biotin, and transduced immune cells or iPSCs can be selected using anti-biotin magnetic beads, which are commercially available (such as from Miltenyi Biotec). Co-expression of huEGFRt also allows for in vivo tracking of adoptively transferred CAR-expressing cells. Furthermore, binding of cetuximab to cells expressing huEGFRt induces cytotoxicity of ADCC effector cells, thereby providing a mechanism to eliminate transduced cells in vivo (Wang et al., Blood 118(5):1255-1263, 2011), such as at the conclusion of therapy.

Also provided herein are S2 subunit-specific monoclonal antibodies (such as a nanobody disclosed herein) modified to enable their use with a universal CAR system. Universal CAR systems increase CAR flexibility and expand their use to additional antigens. Currently, for each patient who receives CAR T cell therapy, autologous T cells are cultured, expanded, and modified to express an antigen-specific CAR. This process is lengthy and expensive, limiting its use. Universal CARs are based on a system in which the signaling components of the CAR are split from the antigen-binding portion of the molecule, but come together using a “lock-key” system. For example, biotin-binding immune receptor (BBIR) CARs are comprised of an intracellular T cell signaling domain fused to an extracellular domain comprising avidin. Biotinylated antigen-specific (such as S2 subunit-specific) monoclonal antibodies can then bind the BBIR to direct immune cells or iPSCs to tumor antigen-expressing cells. Another example is the split, universal and programmable (SUPRA) CAR system. In the SUPRA system, the CAR includes the intracellular signaling domains fused to an extracellular leucine zipper, which is paired with an antigen-specific monoclonal antibody fused to a cognate leucine zipper. For a review of universal CAR systems, see, for example, Zhao et al., J Hematol Oncol 11(1):132, 2018; and Cho et al., Cell 173:1426-1438, 2018. In some aspects herein, the S2 subunit-specific monoclonal antibody is fused to one component of a specific binding pair. In some examples, the monoclonal antibody is fused to a leucine zipper or biotin.

Another type of universal CAR can be generated using a sortase enzyme. A sortase is a prokaryotic enzyme that modifies surface proteins by recognizing and cleaving a carboxyl-terminal sorting signal. Sortase catalyzes transpeptidation between a sortase recognition motif and a sortase acceptor motif. Thus, antigen-specific CARs can be generated by contacting an antigen-specific antibody fused to a sortase recognition motif with a portion of a CAR molecule that includes the intracellular signaling domain(s), a transmembrane region and an extracellular portion comprising a sortase acceptor motif. In the presence of the sortase enzyme, the two components become covalently attached to form a complete antigen-specific CAR. Accordingly, in some aspects herein, an S2 subunit-specific monoclonal antibody is modified to include a sortase recognition motif (see, for example, PCT Publication No. WO 2016/014553).

In some aspects, the S2 subunit-targeted CAR is expressed in allogeneic immune cells (such as T cells, B cells, NK cells, or macrophages), such as allogeneic immune cells from a healthy donor(s). In some examples, the allogeneic cells are genetically engineered to express the S2 subunit-targeted CAR, for example by disrupting expression of an endogenous T cell receptor by insertion of the CAR (see, for example, MacLeod et al., Mol Ther 25(4): 949-961, 2017). Gene editing can be performed using any appropriate gene editing system, such as CRISPR/Cas9, zinc finger nucleases or transcription activator-like effector nucleases (TALEN).

### V. Immunoconjugates

The disclosed single-domain monoclonal antibodies can be conjugated to a therapeutic agent or effector molecule. Immunoconjugates include, but are not limited to, molecules in which there is a covalent linkage of a therapeutic agent to an antibody. A therapeutic agent is an agent with a particular biological activity directed against a particular target molecule or a cell bearing a target molecule. One of skill in the art will appreciate that therapeutic agents can include various drugs such as vinblastine, daunomycin and the like, cytotoxins such as native or modified Pseudomonas exotoxin or diphtheria toxin, encapsulating agents (such as liposomes) that contain pharmacological compositions, radioactive agents such as ¹²⁵I, ³²P, ¹⁴C, ³H and ³⁵S, photon absorbers such as IR700, and other labels, target moieties and ligands.

The choice of a particular therapeutic agent depends on the particular target molecule or cell, and the desired biological effect. Thus, for example, the therapeutic agent can be a cytotoxin that is used to bring about the death of a particular target cell (such as a coronavirus-infected cell). Conversely, where it is desired to invoke a non-lethal biological response, the therapeutic agent can be conjugated to a non-lethal pharmacological agent or a liposome containing a non-lethal pharmacological agent.

With the therapeutic agents and antibodies described herein, one of skill can readily construct a variety of clones containing functionally equivalent nucleic acids, such as nucleic acids which differ in sequence but which encode the same effector moiety or antibody sequence. Thus, the present disclosure provides nucleic acids encoding antibodies and conjugates and fusion proteins thereof.

Effector molecules can be linked to an antibody of interest using any number of means known to those of skill in the art. Both covalent and noncovalent attachment means may be used. The procedure for attaching an effector molecule to an antibody varies according to the chemical structure of the effector. Polypeptides typically contain a variety of functional groups; such as carboxylic acid (COOH), free amine (—NH₂) or sulfhydryl (—SH) groups, which are available for reaction with a suitable functional group on an antibody to result in the binding of the effector molecule. Alternatively, the antibody is derivatized to expose or attach additional reactive functional groups. The derivatization may involve attachment of any of a number of known linker molecules. The linker can be any molecule used to join the antibody to the effector molecule. The linker is capable of forming covalent bonds to both the antibody and to the effector molecule. Suitable linkers are well-known to those of skill in the art and include, but are not limited to, straight or branched-chain carbon linkers, heterocyclic carbon linkers, or peptide linkers. Where the antibody and the effector molecule are polypeptides, the linkers may be joined to the constituent amino acids through their side groups (such as through a disulfide linkage to cysteine) or to the alpha carbon amino and carboxyl groups of the terminal amino acids.

In some circumstances, it is desirable to free the effector molecule from the antibody when the immunoconjugate has reached its target site. Therefore, in these circumstances, immunoconjugates include linkages that are cleavable in the vicinity of the target site. Cleavage of the linker to release the effector molecule from the antibody may be prompted by enzymatic activity or conditions to which the immunoconjugate is subjected either inside the target cell or in the vicinity of the target site.

In view of the large number of methods that have been reported for attaching a variety of radiodiagnostic compounds, radiotherapeutic compounds, labels (such as enzymes or fluorescent molecules), drugs, toxins, and other agents to antibodies, one skilled in the art can determine a suitable method for attaching a given agent to an antibody or other polypeptide.

The antibodies disclosed herein can be derivatized or linked to another molecule (such as another peptide or protein). In general, the antibodies or portion thereof is derivatized such that the binding to the target antigen is not affected adversely by the derivatization or labeling. For example, the antibody can be functionally linked (by chemical coupling, genetic fusion, noncovalent association or otherwise) to one or more other molecular entities, such as another antibody (for example, a bispecific antibody or a diabody), a detection agent, a photon absorber, a pharmaceutical agent, and/or a protein or peptide that can mediate association of the antibody or antibody portion with another molecule (such as a streptavidin core region or a polyhistidine tag).

One type of derivatized antibody is produced by cross-linking two or more antibodies (of the same type or of different types, such as to create bispecific antibodies). Suitable crosslinkers include those that are heterobifunctional, having two distinctly reactive groups separated by an appropriate spacer (such as m-maleimidobenzoyl-N-hydroxysuccinimide ester) or homobifunctional (such as disuccinimidyl suberate). Such linkers are commercially available.

The antibody provided herein can be conjugated with a detectable marker; for example, a detectable marker capable of detection by ELISA, spectrophotometry, flow cytometry, microscopy or diagnostic imaging techniques (such as computed tomography (CT), computed axial tomography (CAT) scans, magnetic resonance imaging (MRI), nuclear magnetic resonance imaging NMRI), magnetic resonance tomography (MTR), ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). For example, useful detectable markers include fluorescent compounds, including fluorescein, fluorescein isothiocyanate, rhodamine, 5-dimethylamine-1-napthalenesulfonyl chloride, phycoerythrin, lanthanide phosphors and the like. Bioluminescent markers are also of use, such as luciferase, green fluorescent protein (GFP) and yellow fluorescent protein (YFP). An antibody can also be conjugated with enzymes that are useful for detection, such as horseradish peroxidase, β-galactosidase, luciferase, alkaline phosphatase, glucose oxidase and the like. When an antibody or antigen binding fragment is conjugated with a detectable enzyme, it can be detected by adding additional reagents that the enzyme uses to produce a reaction product that can be discerned. For example, when the agent horseradish peroxidase is present the addition of hydrogen peroxide and diaminobenzidine leads to a colored reaction product, which is visually detectable. An antibody or antigen binding fragment may also be conjugated with biotin, and detected through indirect measurement of avidin or streptavidin binding. The avidin itself can also be conjugated with an enzyme or a fluorescent label.

An antibody provided herein may be labeled with a magnetic agent, such as gadolinium. Antibodies can also be labeled with lanthanides (such as europium and dysprosium), and manganese. Paramagnetic particles such as superparamagnetic iron oxide are also of use as labels. An antibody may also be labeled with a predetermined polypeptide epitopes recognized by a secondary reporter (such as leucine zipper pair sequences, binding sites for secondary antibodies, metal binding domains, epitope tags). In some aspects, labels are attached by spacer arms of various lengths to reduce potential steric hindrance.

An antibody provided herein can also be labeled with a radiolabeled amino acid. The radiolabel may be used for both diagnostic and therapeutic purposes. For instance, the radiolabel may be used to detect expression of a target antigen by x-ray, emission spectra, or other diagnostic techniques. Examples of labels for polypeptides include, but are not limited to, the following radioisotopes or radionucleotides: ³H, ¹⁴C, ¹⁵N, ³⁵S, ⁹⁰Y ⁹⁹Tc, ¹¹¹In, ¹²⁵I, ¹³¹I.

An antibody disclosed herein can also be conjugated to a photon absorber. In some aspects, the photon absorber is a phthalocyanine dye, such as, but not limited to, IRDye® 700DX (also known as “IR700”). Antibody-photoabsorber conjugates can be used for photoimmunotherapy (for example to kill cells infected with a coronavirus, such as SARS-CoV and/or SARS-CoV-2).

An antibody can also be derivatized with a chemical group such as polyethylene glycol (PEG), a methyl or ethyl group, or a carbohydrate group. These groups may be useful to improve the biological characteristics of the antibody, such as to increase serum half-life or to increase tissue binding.

Toxins can be employed with the monoclonal antibodies described herein to produce immunotoxins. Exemplary toxins include ricin, abrin, diphtheria toxin and subunits thereof, as well as botulinum toxins A through F. These toxins are readily available from commercial sources (for example, Sigma Chemical Company, St. Louis, MO). Contemplated toxins also include variants of the toxins described herein (see, for example, see, U.S. Pat. Nos. 5,079,163 and 4,689,401). In one aspect, the toxin is Pseudomonas exotoxin (PE) (U.S. Pat. No. 5,602,095). As used herein “Pseudomonas exotoxin” refers to a full-length native (naturally occurring) PE or a PE that has been modified. Such modifications can include, but are not limited to, elimination of domain Ia, various amino acid deletions in domains Ib, II and III, single amino acid substitutions and the addition of one or more sequences at the carboxyl terminus (for example, see Siegall et al., J. Biol. Chem. 264:14256-14261, 1989).

PE employed with the monoclonal antibodies described herein can include the native sequence, cytotoxic fragments of the native sequence, and conservatively modified variants of native PE and its cytotoxic fragments. Cytotoxic fragments of PE include those which are cytotoxic with or without subsequent proteolytic or other processing in the target cell. Cytotoxic fragments of PE include PE40, PE38, and PE35. For additional description of PE and variants thereof, see for example, U.S. Pat. Nos. 4,892,827; 5,512,658; 5,602,095; 5,608,039; 5,821,238; and 5,854,044; U.S. Patent Application Publication No. 2015/0099707; PCT Publication Nos. WO 99/51643 and WO 2014/052064; Pai et al., Proc. Natl. Acad. Sci. USA 88:3358-3362, 1991; Kondo et al., J. Biol. Chem. 263:9470-9475, 1988; Pastan et al., Biochim. Biophys. Acta 1333:C1-C6, 1997.

Also contemplated herein are protease-resistant PE variants and PE variants with reduced immunogenicity, such as, but not limited to PE-LR, PE-6X, PE-8X, PE-LR/6X and PE-LR/8X (see, for example, Weldon et al., Blood 113(16):3792-3800, 2009; Onda et al., Proc Natl Acad Sci USA 10⁵(32):11311-11316, 2008; and PCT Publication Nos. WO 2007/016150, WO 2009/032954 and WO 2011/032022).

In some examples, the PE is a variant that is resistant to lysosomal degradation, such as PE-LR (Weldon et al., Blood 113(16):3792-3800, 2009; PCT Publication No. WO 2009/032954). In other examples, the PE is a variant designated PE-LR/6X (PCT Publication No. WO 2011/032022). In other examples, the PE variant is PE with reducing immunogenicity. In yet other examples, the PE is a variant designated PE-LR/8M (PCT Publication No. WO 2011/032022).

Modification of PE may occur in any previously described variant, including cytotoxic fragments of PE (for example, PE38, PE-LR and PE-LR/8M). Modified PEs may include any substitution(s), such as for one or more amino acid residues within one or more T-cell epitopes and/or B cell epitopes of PE, or deletion of one or more T-cell and/or B-cell epitopes (see, for example, U.S. Patent Application Publication No. 2015/0099707).

Contemplated forms of PE also include deimmunized forms of PE, for example versions with domain II deleted (for example, PE24). Deimmunized forms of PE are described in, for example, PCT Publication Nos. WO 2005/052006, WO 2007/016150, WO 2007/014743, WO 2007/031741, WO 2009/32954, WO 2011/32022, WO 2012/154530, and WO 2012/170617.

The antibodies described herein can also be used to target any number of different diagnostic or therapeutic compounds to cells expressing coronavirus S protein on their surface (e.g., SARS-CoV-2 or SARS-CoV infected cells). Thus, an antibody of the present disclosure can be attached directly or via a linker to a drug that is to be delivered directly to cells expressing coronavirus spike protein. This can be done for therapeutic, diagnostic or research purposes. Therapeutic agents include such compounds as nucleic acids, proteins, peptides, amino acids or derivatives, glycoproteins, radioisotopes, photon absorbers, lipids, carbohydrates, or recombinant viruses. Nucleic acid therapeutic and diagnostic moieties include antisense nucleic acids, derivatized oligonucleotides for covalent cross-linking with single or duplex DNA, and triplex forming oligonucleotides.

Alternatively, the molecule linked to an antibody can be an encapsulation system, such as a nanoparticle, liposome or micelle that contains a therapeutic composition such as a drug, a nucleic acid (for example, an antisense nucleic acid), or another therapeutic moiety that is preferably shielded from direct exposure to the circulatory system. Means of preparing liposomes attached to antibodies are well known to those of skill in the art (see, for example, U.S. Pat. No. 4,957,735; Connor et al., Pharm. Ther. 28:341-365, 1985).

Antibodies described herein can also be covalently or non-covalently linked to a detectable label. Detectable labels suitable for such use include any composition detectable by spectroscopic, photochemical, biochemical, immunochemical, electrical, optical or chemical means. Useful labels include magnetic beads, fluorescent dyes (for example, fluorescein isothiocyanate, Texas red, rhodamine, green fluorescent protein, and the like), radiolabels (for example, ³H, ¹²⁵I, ³⁵S, ¹⁴C, or ³²P), enzymes (such as horseradish peroxidase, alkaline phosphatase and others commonly used in an ELISA), and colorimetric labels such as colloidal gold or colored glass or plastic (such as polystyrene, polypropylene, latex, and the like) beads.

Means of detecting such labels are known. Thus, for example, radiolabels may be detected using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

### VI. Antibody-Drug Conjugates (ADCs)

ADCs are compounds comprised of an antigen-specific antibody (such as a single-domain antibody or antigen-binding fragment of an immunoglobulin) and a drug, for example an anti-viral agent (such as remdesivir, galidesivir, arbidol, favipiravir, baricitinib, molnupiravir, or lopinavir/ritonavir) or a cytotoxic agent (such as an anti-microtubule agent or cross-linking agent). Because ADCs are capable of specifically targeting cells expressing a particular antigen, the drug can be much more potent than agents used for standard systemic therapy. For example, cytotoxic drugs used with ADCs typically have an IC₅₀ that is 100- to 1000-fold more potent than conventional chemotherapeutic agents. Exemplary cytotoxic drugs include anti-microtubule agents, such as maytansinoids and auristatins (such as auristatin E and auristatin F). Other cytotoxins for use with ADCs include pyrrolobenzodiazepines (PBDs), which covalently bind the minor groove of DNA to form interstrand crosslinks. In some instances, ADCs include a 1:2 to 1:4 ratio of antibody provided herein to drug (Bander, Clinical Advances in Hematology & Oncology 10(8; suppl 10):3-7, 2012).

The antibody and drug can be linked by a cleavable or non-cleavable linker. However, in some instances, it is desirable to have a linker that is stable in the circulation to prevent systemic release of the cytotoxic drug that could result in significant off-target toxicity. Non-cleavable linkers prevent release of the cytotoxic agent before the ADC is internalized by the target cell. Once in the lysosome, digestion of the antibody by lysosomal proteases results in the release of the cytotoxic agent (Bander, Clinical Advances in Hematology & Oncology 10(8; suppl 10):3-7, 2012).

One method for site-specific and stable conjugation of a drug to a monoclonal antibody (or a V_{H}H-Fc protein) is via glycan engineering. Monoclonal antibodies have one conserved N-linked oligosaccharide chain at the Asn297 residue in the CH2 domain of each heavy chain (Qasba et al., Biotechnol Prog 24:520-526, 2008). Using a mutant β1,4-galactosyltransferase enzyme (Y289L-Gal-T1; U.S. Patent Application Publication Nos. 2007/0258986 and 2006/0084162), 2-keto-galactose is transferred to free GlcNAc residues on the antibody heavy chain to provide a chemical handle for conjugation.

The oligosaccharide chain attached to monoclonal antibodies can be classified into three groups based on the terminal galactose residues—fully galactosylated (two galactose residues; IgG-G2), one galactose residue (IgG-G1) or completely degalactosylated (IgG-G0). Treatment of a monoclonal antibody with (1,4-galactosidase converts the antibody to the IgG-G0 glycoform. The mutant β1,4-galactosyltransferase enzyme can transfer 2-keto-galactose or 2-azido-galactose from their respective UDP derivatives to the GlcNAc residues on the IgG-G1 and IgG-G0 glycoforms. The chemical handle on the transferred sugar enables conjugation of a variety of molecules to the monoclonal antibody via the glycan residues (Qasba et al., Biotechnol Prog 24:520-526, 2008).

Provided herein are ADCs that include a drug (such as an anti-viral agent) conjugated to a monoclonal antibody that binds (such as specifically binds) the S2 subunit of coronavirus S protein. In some aspects, the drug is a small molecule. In some examples, the drug is an anti-viral agent, such as remdesivir, galidesivir, arbidol, favipiravir, baricitinib, molnupiravir, or lopinavir/ritonavir. In some examples, the drug is a cross-linking agent, an anti-microtubule agent and/or anti-mitotic agent, or any cytotoxic agent suitable for mediating killing of tumor cells. Exemplary cytotoxic agents include, but are not limited to, a PBD, an auristatin, a maytansinoid, dolastatin, calicheamicin, nemorubicin and its derivatives, PNU-159682, anthracycline, vinca alkaloid, taxane, trichothecene, CC1065, camptothecin, elinafide, a combretastain, a dolastatin, a duocarmycin, an enediyne, a geldanamycin, an indolino-benzodiazepine dimer, a puromycin, a tubulysin, a hemiasterlin, a spliceostatin, or a pladienolide, as well as stereoisomers, isosteres, analogs, and derivatives thereof that have cytotoxic activity.

In some aspects, the ADC can further include a linker. In some examples, the linker is a bifunctional or multifunctional moiety that can be used to link one or more drug moieties to an antibody to form an ADC. In some aspects, ADCs are prepared using a linker having reactive functionalities for covalently attaching to the drug and to the antibody. For example, a cysteine thiol of an antibody can form a bond with a reactive functional group of a linker or a drug-linker intermediate to make an ADC.

In some examples, a linker has a functionality that is capable of reacting with a free cysteine present on an antibody to form a covalent bond. Exemplary linkers with such reactive functionalities include maleimide, haloacetamides, α-haloacetyl, activated esters such as succinimide esters, 4-nitrophenyl esters, pentafluorophenyl esters, tetrafluorophenyl esters, anhydrides, acid chlorides, sulfonyl chlorides, isocyanates, and isothiocyanates.

In some examples, a linker has a functionality that can react with an electrophilic group present on an antibody. Examples of such electrophilic groups include, but are not limited to, aldehyde and ketone carbonyl groups. In some cases, a heteroatom of the reactive functionality of the linker can react with an electrophilic group on an antibody and form a covalent bond to an antibody unit. Non-limiting examples include hydrazide, oxime, amino, hydrazine, thiosemicarbazone, hydrazine carboxylate and arylhydrazide.

In some examples, the linker is a cleavable linker, which facilitates release of the drug. Examples of cleavable linkers include acid-labile linkers (for example, comprising hydrazone), protease-sensitive linkers (for example, peptidase-sensitive), photolabile linkers, and disulfide-containing linkers (Chari et al., Cancer Res 52:127-131, 1992; U.S. Pat. No. 5,208,020).

The ADCs disclosed herein can be used for the treatment of a coronavirus infection alone or in combination with another therapeutic agent and/or in combination with any standard therapy for the treatment of a coronavirus infection, such as COVID-19 (e.g., remdesivir, galidesivir, lenzilumab, molnupiravir (Merck), hydroxychloroquine, dexamethasone, arbidol, favipiravir, baricitinib, lopinavir/ritonavir, zinc ions, and interferon beta-tb).

### VII. Multi-Specific Antibodies

Multi-specific antibodies are recombinant proteins comprised of two or more monoclonal antibodies (such as single-domain antibodies) or antigen-binding fragments of two or more different monoclonal antibodies. For example, bispecific antibodies are comprised of two different monoclonal antibodies or antigen-binding fragments thereof. Thus, bispecific antibodies bind two different antigens or epitopes and trispecific antibodies bind three different antigens or epitopes. Multi-specific antibodies can be used for treating a coronavirus infection by simultaneously targeting, for example, a coronavirus S protein (including N-terminal domain (NTD), receptor binding domain (RBD), or whole S1 or S2 subunits) and a carbohydrate (including N-glycans), envelope protein, or hemagglutinin-esterase dimer (HE). In some examples, the multi-specific antibody includes a first binding domain that targets a portion of a coronavirus S protein (such as the NTD, RBD, S1 subunit or S2 subunit) and a second binding domain that targets a different portion of the same coronavirus S protein (such as the NTD, RBD, S1 subunit or S2 subunit). For example, monoclonal antibodies that bind the S1 subunit of the spike protein (such as the RBD) include bamlanivimab, etesevimab, casirivimab, imdevimab, and sotrovimab. Monoclonal antibodies that bind the NTD include, for example, 4A8 (Chi et al., Science 369:650-655, 2020). In a specific example, the multi-specific antibody includes a first binding domain that targets a coronavirus S1 subunit (such as a S1 subunit-specific monoclonal antibody, such as bamlanivimab, etesevimab, casirivimab, imdevimab, sotrovimab), and a second binding domain that targets a coronavirus S2 subunit (such as an S2 subunit-specific monoclonal antibody, such as one provided herein).

In some aspects, the multi-specific antibodies include a monoclonal antibody that specifically binds a SARS-CoV-2 S2 subunit and an immune cell engager, for example, a T cell engager (e.g., an antibody that specifically binds CD3) or an NK cell engager (e.g., an antibody that specifically binds NKp46 or CD16). The S2 subunit-specific single-domain monoclonal antibodies disclosed herein can be used to generate multi-specific (such as bispecific or trispecific) antibodies that target both spike protein and CTLs, or target both spike protein and NK cells, thereby providing a means to treat coronavirus-infected cells.

Bi-specific T-cell engagers (BiTEs) are a type of bispecific monoclonal antibody that are fusions of a first monoclonal antibody (such as a scFv or a single-domain antibody) that targets a specific antigen (such as the S2 subunit of a SARS-CoV-2 spike protein) and a second antibody that binds T cells, such as CD3 on T cells.

Bi-specific killer cell engagers (BiKEs) are a type of bispecific monoclonal antibody that are fusions of a first monoclonal antibody (such as a scFv or single-domain antibody) that targets a specific antigen (such as the S2 subunit of a SARS-CoV-2 spike protein) and a second scFv that binds a NK cell activating receptor, such as CD16.

Provided herein are multi-specific, such as trispecific or bispecific, monoclonal antibodies comprising an S2 subunit-specific monoclonal antibody. In some aspects, the multi-specific monoclonal antibody further comprises a monoclonal antibody that specifically binds S protein RBD, NTD, or S1 subunit, or a carbohydrate (such as an N-glycan), or other viral proteins (such as envelope or HE). In other aspects, the multi-specific monoclonal antibody includes an S2 subunit-specific monoclonal antibody and an immune cell engager. Also provided are isolated nucleic acid molecules and vectors encoding the multi-specific antibodies, and host cells comprising the nucleic acid molecules or vectors. Multi-specific antibodies comprising an S2 subunit-specific antibody can be used for the treatment of a coronavirus infection. Thus, provided herein are methods of treating a subject with a coronavirus infection by administering to the subject a therapeutically effective amount of the S2 subunit-targeting multi-specific antibody.

### VIII. Antibody-Nanoparticle Conjugates

The monoclonal antibodies disclosed herein can be conjugated to a variety of different types of nanoparticles to deliver cytotoxic agents or anti-viral agents (such as remdesivir, galidesivir, arbidol, favipiravir, baricitinib, molnupiravir, or lopinavir/ritonavir) directly to coronavirus-infected cells via binding of the antibody to the spike protein expressed on the surface of infected cells. The use of nanoparticles reduces off-target side effects and can also improve drug bioavailability and reduce the dose of a drug required to achieve a therapeutic effect. Nanoparticle formulations can be tailored to suit the drug that is to be carried or encapsulated within the nanoparticle. For example, hydrophobic molecules can be incorporated inside the core of a nanoparticle, while hydrophilic drugs can be carried within an aqueous core protected by a polymeric or lipid shell. Examples of nanoparticles include, but are not limited to, nanospheres, nanocapsules, liposomes, dendrimers, polymeric micelles, niosomes, and polymeric nanoparticles (Fay and Scott, Immunotherapy 3(3):381-394, 2011).

Liposomes are common types of nanoparticles used for drug delivery. An antibody conjugated to a liposome is often referred to as an “immunoliposome.” The liposomal component of an immunoliposome is typically a lipid vesicle of one or more concentric phospholipid bilayers. In some cases, the phospholipids are composed of a hydrophilic head group and two hydrophobic chains to enable encapsulation of both hydrophobic and hydrophilic drugs. Conventional liposomes are rapidly removed from the circulation via macrophages of the reticuloendothelial system (RES). To generate long-circulating liposomes, the composition, size and charge of the liposome can be modulated. The surface of the liposome may also be modified, such as with a glycolipid or sialic acid. For example, the inclusion of polyethylene glycol (PEG) significantly increases circulation half-life. Liposomes for use as drug delivery agents, including for preparation of immunoliposomes, have been described in the art (see, for example, Paszko and Senge, Curr Med Chem 19(31)5239-5277, 2012; Immordino et al., Int J Nanomedicine 1(3):297-315, 2006; U.S. Patent Application Publication Nos. 2011/0268655; 2010/00329981).

Niosomes are non-ionic surfactant-based vesicles having a structure similar to liposomes. The membranes of niosomes are composed only of nonionic surfactants, such as polyglyceryl-alkyl ethers or N-palmitoylglucosamine. Niosomes range from small, unilamellar to large, multilamellar particles. These nanoparticles are monodisperse, water-soluble, chemically stable, have low toxicity, are biodegradable and non-immunogenic, and increase bioavailability of encapsulated drugs.

Dendrimers include a range of branched polymer complexes. These nanoparticles are water-soluble, biocompatible and are sufficiently non-immunogenic for human use. Generally, dendrimers consist of an initiator core, surrounded by a layer of a selected polymer that is grafted to the core, forming a branched macromolecular complex. Dendrimers are typically produced using polymers such as poly(amidoamine) or poly(L-lysine). Dendrimers have been used for a variety of therapeutic and diagnostic applications, including for the delivery of DNA, RNA, bioimaging contrast agents, chemotherapeutic agents and other drugs.

Polymeric micelles are composed of aggregates of amphiphilic co-polymers (consisting of both hydrophilic and hydrophobic monomer units) assembled into hydrophobic cores, surrounded by a corona of hydrophilic polymeric chains exposed to the aqueous environment. In many cases, the polymers used to prepare polymeric micelles are heterobifunctional copolymers composed of a hydrophilic block of PEG, poly(vinyl pyrrolidone) and hydrophobic poly(L-lactide) or poly(L-lysine) that forms the particle core. Polymeric micelles can be used to carry drugs that have poor solubility. These nanoparticles have been used to encapsulate a number of drugs, including doxorubicin and camptothecin. Cationic micelles have also been developed to carry DNA or RNA molecules.

Polymeric nanoparticles include both nanospheres and nanocapsules. Nanospheres consist of a solid matrix of polymer, while nanocapsules contain an aqueous core. The formulation selected typically depends on the solubility of the therapeutic agent to be carried/encapsulated; poorly water-soluble drugs are more readily encapsulated within nanospheres, while water-soluble and labile drugs, such as DNA and proteins, are more readily encapsulated within nanocapsules. The polymers used to produce these nanoparticles include, for example, poly(acrylamide), poly(ester), poly(alkylcyanoacrylates), poly(lactic acid) (PLA), poly(glycolic acids) (PGA), and poly(D,L-lactic-co-glycolic acid) (PLGA).

Antibodies provided herein can be conjugated to a suitable nanoparticle according to standard methods. For example, conjugation can be either covalent or non-covalent. In some aspects in which the nanoparticle is a liposome, the antibody is attached to a sterically stabilized, long circulation liposome via a PEG chain. Coupling of antibodies or antibody fragments to a liposome can also involve thioester bonds, for example by reaction of thiols and maleimide groups. Cross-linking agents can be used to create sulfhydryl groups for attachment of antibodies to nanoparticles (Paszko and Senge, Curr Med Chem 19(31)5239-5277, 2012).

### IX. Nucleic Acid Molecules

Nucleic acid molecules (for example, DNA, cDNA, mIRNA, or RNA molecules) encoding the amino acid sequences of the disclosed antibodies, fusion proteins, and conjugates that specifically bind to an S2 subunit of coronavirus spike protein, are provided. Nucleic acid molecules encoding these molecules can readily be produced using the amino acid sequences provided herein (such as the CDR sequences and the V_{H}H or VNAR sequences), sequences available in the art (such as framework or constant region sequences), and the genetic code. In some aspects, the nucleic acid molecules can be expressed in a host cell (such as a mammalian cell, yeast cell or a bacterial cell) to produce a disclosed antibody, fusion protein or antibody conjugate (e.g., CAR, immunotoxin, multi-specific antibody). In some examples, the nanobody is in an IgG, IgA or IgM format.

Provided below are exemplary nucleic acid sequences of the disclosed shark and camel nanobodies specific for the S2 subunit of SARS-CoV-2 spike protein.

NCI-CoV-S2A9
(SEQ ID NO: 13)
GCTCGAGTGGACCAAACACCGAGATCAGTAACAAAGGAGACGGGCGAATCACTGACCATCAA
CTGTGTCCTACGAGATGCGAGCTATGCATTGGGCAGGACGTGCTGGTATCGAAAAAAATCGG
GCTCAACAAACGAGGAGAGCATATCGAAAGGTGGACGATATGTTGAAACAGTTAACAGCGGA
TCAAAGTCCTTTTCTTTGAGAATTAATGATCTAACAGTTGAAGACGGTGGCACGTATCGTTG
CGGTGTCTCGACGCCCTGGTCGTGTGACTACGGGCCGCTGTGCTTCTATGCTGCATGCGGAG
ATGGCACTGTCGTGACTGTGAAT
NCI-CoV-S2G8
(SEQ ID NO: 14)
GCTCGAGTGGACCAAACACCGCAAACAATAACAAAGGAGACGGGCGAATCACTGACCATCAA
CTGTGTCCTACGAGATAGTAACTGTCCATTGTCCAACACCTACTGGTATCGCAAAAAATCGG
GCTCAACAAACGAGGAGAGCATATCGAAAGGTGGACGATATGTTGAAACAGTTAACAGCGGA
TCAAAGTCCTTTTCTTTGAGAATTAATGATCTAACAGTTGAAGACAGTGGCACGTATCGATG
CAAGGTCCGAACTCACTGGTCGATATGCTGGGAGATGGGACTGGAGCCCAACTATGATGTAT
ACGGAGGTGGCACTGTCGTGACTGTGAAT
NCI-CoV-S3A10
(SEQ ID NO: 15)
GCTCGAGTGGACCAAACACCGAGATCAGTAACAAAGGAGACGGGCGAATCACTGACCATCAA
CTGTGTCCTACGAGATGCGAGCTATGCATTGGGCAGCACGTGCTGGTATCGAAAAAAATCGG
GCTCAACAAACGAGGAGAGCATATCGAAAGGTGGACGATATGTTGAAACAGTTAACAGCGGA
TCAAAGTCCTTTTCTTTGAGAATTAATGATCTAACAGTTGAAGACGGTGGCACGTATCGTTG
CGGTCGGATCCAAAACACGAAAGCTGTGCTTCTCCCATGTGTCTATGCTGCATGCGGAGATG
GCACTGTCGTGACTGTGAATAGTGGCCAGGCCGGCCACCACCACCACCACCACGGCGACTAC
AAGGACGATGACGATAAGGG
NCI-CoV-S4A9
(SEQ ID NO: 16)
GCTCGAGTGGACCAAACACCGAGATCAGTAACAAAGGAGACGGGCGAATCACTGACCATCAA
CTGTGTCCTACGAGATGCGAGCTATGCATTGGGCAGCACGTGCTGGTATCGAAAAAAATCGG
GCTCAACAAACGAGGAGAGCATATCGAAAGGTGGACGATATGTTGAAACAGTTAACAGCGGA
TCAAAGTCCTTTTCTTTGAGAATTAATGATCTAACAGTTGAAGACGGTGGCACGTATCGTTG
CGGTGTCCAAAGCAGGTGGGGAGCTGTGCCCAAGCTGTGCTCTAACTATGCTGCATGCGGAG
ATGGCACTGCCGTGACTGTGAATAGTGGCCAGGCCGGCCACCACCACCACCACCACGGCGAC
TACAAGGACGATGACGATAAGGG
NCI-CoV-S4C12
(SEQ ID NO: 17)
GCTCGAGTGGACCAAACACCGCAAACAATAACAAAGGAGACGGGCGAATCACTGACCATCAA
CTGTGTCCTACGAGATAGTAACTGTGCATTGTCCAGCACGTACTGGTATCGCAAAAAATCGG
GCTCAACAAACGAGGAGAGCATATCGAAAGGTGGACGATATGTTGAAACAGTTAACAGCGGA
TCAAAGTCCTTTTCTTTGAGAATTAATGATCTAACAGTTGAAGACAGTGGCACGTATCGATG
CAAGGTAGGCTGCTGGTACGGCTATGAGTCGTCACTCATGAACTGGAGCAGGGTATACGGAG
GTGGCACTGCCGTGACTGTGAAT
NCI-CoV-S6E1
(SEQ ID NO: 18)
GCTCGAGTGGACCAAACACCGAGATCAGTAACAAAGGAGACGGGCGAATCACTGACCATCAA
CTGTGTCCTACGAGATGCGAGCTATGCATTGGGCAGCACGTGCTGGTATCGAAAAAAATCGG
GCTCAACAAACGAGGAGAGCATATCGAAAGGTGGACGATATGTTGAAACAGTTAACAGCGGA
TCAAAGTCCTTTTCTTTGAGAATTAATGATCTAACAGTTGAAGACAGTGGCACGTATCGATG
CAAGGGGTACACGCCCTGTATGCCCGACTGGGTCCCCGATGTATACGGAATGGGCACTGTCG
TGACTGTGAAT
NCI-CoV-CLA2
(SEQ ID NO: 19)
CAGGTGCAGCTGGTGGAGTCTGGCGGAGGATCCGTGCAGCCAGGAGGAAGCCTGAGGCTGTC
TTGCGCCGTGTCCGGCTACACCTCCAGCTCTTATTGTTTCGGCTGGTTTAGACAGGCTCCAG
GCAAGGACAGGGAGGGAATCATCGCTATGGAGCCAGGAAACACCTACACAGCTGGAGCTGTG
AAGGGCAGGTTCACCATCAGCCGGGATAACGCCAAGAATATGGTGTATCTGCAGATGAACTC
TCTGAAGCCTGACGATACAGCTATGTACTATTGCGCTGCTTCCCCATACCCTGGAACCTGGC
TGAATCCAGACCTGTTTGATACATGGGGCCAGGGCACCCAGGTGACAGTGTCCAGC
NCI-CoV-CG2G2
(SEQ ID NO: 20)
GCCGTGCAGCTGGTGGAGAGCGGCGGAGGATCTGTGCCACCTGGAGGCTCCCTGACCCTGAG
CTGCACAGTGTCTGACAACATCTTCCGCACCTACGCCGTGGGCTGGTTTCGCCAGGCTCCAG
AGAAGAGGTGTCAGTGGATCGCCACCCTGAACGCTGACGAGAGCACAGAGTACAATGTGGCC
GTGACCGGCAGGTTCACAATCTCTCGGGATAATTCTGGCAGATCCGTGTATCTGCAGATGGA
CAACCTGAATTCTGAGGATACAGCTATCTACTATTGCGCCTCCCTGGCTCGGAAGTGGTCCT
GTCACGATAGCTGGGGCAGAGGCACCCAGGTGACAGTGTCCAGC
NCI-CoV-CGH3
(SEQ ID NO: 21)
GCCGTGCAGCTAGTTGAGAGCGGAGGCGGTCTTGTCCAACCAGGAGGGAGCCTGAGACTGTC
TTGTGCCGCATCCGGCTTCACATTCTCATCCTATGCTATGGGGTGGGTGCGGCAGGCCCCTG
GAAAAGGTCTGGAATGGATATCCGCAATCAATAGTGGAGGCGACATTCGATATTACGCTGAC
AGTGTCAAGGGCAGGTTTACAATCTCTCGCGACAATGCGAAGAACACCCTGTATTTACAAAT
GAACTCTCTCAAACCCGAAGATACCGCTGTGTACTACTGCGCCACTGATGATGAGATTTTGT
CGTACGGGCTCGGGACCTCATTTGACTATTGGGGTCAGGGCACTCAGGTAACGGTGTCCAGC
NCI-CoV-CGF10
(SEQ ID NO: 22)
GCCGTGCAGCTTGTGGAGAGTGGCGGGGGCCTGGTCCAACCAGGAGGAAGTCTGCGGCTCTC
CTGCGCCGCCTCTGGGTTCACATTTTCCAGCTATGGAATGCACTGGGTAAGACAGGCGCCCG
GTAAGGGGCTCGAACGTGTGGCAGGTATAAACTGGGATGGGTCTAATTCTTATTACACCGAG
ACGGTTAAAGGCCGCTTCACTATCTCAAGAGACAACGCTAAGAATACAGCTTACCTACAAAT
GAACAGCCTGAAACCTGAAGACACCGCAATGTACTACTGCCGAACATTAATTTTGGACTCGG
ATTGTGATCCGGGCTCAACCTATAGGTATGCCTGGGGACAGGGCACCCAGGTCACTGTTCCA
GC
NCI-CoV-CG2D11
(SEQ ID NO: 23)
GCGGTCCAATTGGTGGATAGCGGCGGCGGGAGCGTCCAGGCTGGAGGGAGTCTGAGGTTATC
TTGCGCAGGAAGCGCATTTACGTACAGTTCCTATTGTATGGGCTGGTTTCGCCAGGCGCCGG
GAAAGGAAAGAGAGGCCGTGGCTGCTATCAACTCCGGGACAAGCTCAACTTATTACGCTGAT
TCAGTGAAGGGTCGGTTCACTGTTTCGCAGGATAACGCCAATAATACCGTATACCTGCTCAT
GATATCTCTTAAACCCGAGGACACAGCCATTTATATCTGCGCCGCCCTGGTGGCCCCTGACT
ATGACGACTACGATGTCGCAGCACCAGACAACTTCGATTACTGGGGTCAGGGCACCCAAGTG
ACCGTTTCCAGC
NCI-CoV-CL3H4
(SEQ ID NO: 24)
GACGTCCAGTTGGTGGAGAGTGGCGGCGGAGCTGTGAAACCCGGAGGATCGCTCACTCTGAC
CTGCACCGTGCAGGAAGGCTCCCGTAATTGCGTTGGTTGGTTCCGCCAGGGCTCCGGTAAAG
ATAGACAAGCAGTGGCCATGATTAACGGCAATGGGGGTACCACAAACTATGACGACAGGGTA
AAGGGAAGATTTCAGATCAGCAGGGACGGCTCTTTAGTCCACCTTCGGATGAACGATGTCAA
GCTGGGTGATACTGATATATACTACTGTCGACAGGGGCAGACATATGATTGTTGGGGGGGCA
CCTGGACGGACAGCGGATGGGGGCAAGGGACACAGGTTACTGTGTCCAGC

The genetic code can be used to construct a variety of functionally equivalent nucleic acid sequences, such as nucleic acids that differ in their sequence but which encode the same antibody sequence, or encode a conjugate or fusion protein including the single-domain antibody sequence. In some aspects, the nucleic acid sequence is at least 80%, at least 85%, at least 90%, at least 95%, at least 96%, at least 97%, at least 98%, or at least 99% identical to any one of SEQ ID NOs: 13-24. In some examples, the nucleic acid sequence comprises or consists of any one of SEQ ID NOs: 13-24, or a degenerate variant thereof.

Nucleic acid molecules encoding the antibodies, fusion proteins, and conjugates that specifically 25 bind to an S2 subunit can be prepared by any suitable method including, for example, cloning of appropriate sequences or by direct chemical synthesis by standard methods. Chemical synthesis produces a single stranded oligonucleotide. This can be converted into double stranded DNA by hybridization with a complementary sequence or by polymerization with a DNA polymerase using the single strand as a template.

Exemplary nucleic acids can be prepared by cloning techniques. Examples of appropriate cloning and sequencing techniques can be found, for example, in Green and Sambrook (Molecular Cloning: A Laboratory Manual, 4^{th} ed., New York: Cold Spring Harbor Laboratory Press, 2012) and Ausubel et al. (Eds.) (Current Protocols in Molecular Biology, New York: John Wiley and Sons, including supplements).

Nucleic acids can also be prepared by amplification methods. Amplification methods include the 35 polymerase chain reaction (PCR), the ligase chain reaction (LCR), the transcription-based amplification system (TAS), and the self-sustained sequence replication system (3SR).

The nucleic acid molecules can be expressed in a recombinantly engineered cell such as in bacterial, plant, yeast, insect or mammalian cells. The antibodies and conjugates can be expressed as individual proteins including the single-domain monoclonal antibody (linked to an effector molecule or detectable marker as needed), or can be expressed as a fusion protein. Any suitable method of expressing and purifying antibodies and antigen binding fragments may be used; non-limiting examples are provided in Al-Rubeai (Ed.), Antibody Expression and Production, Dordrecht; New York: Springer, 2011).

One or more DNA sequences encoding the antibodies, fusion proteins, or conjugates can be expressed in vitro by DNA transfer into a suitable host cell. The cell may be prokaryotic or eukaryotic. Numerous expression systems available for expression of proteins including E. coli, other bacterial hosts, yeast, and various higher eukaryotic cells, for example mammalian cells, such as the COS, CHO, HeLa and myeloma cell lines, can be used to express the disclosed antibodies and antigen binding fragments. Methods of stable transfer, meaning that the foreign DNA is continuously maintained in the host may be used.

The expression of nucleic acids encoding the antibodies and conjugates described herein can be achieved by operably linking the DNA or cDNA to a promoter (which is either constitutive or inducible), followed by incorporation into an expression cassette. The promoter can be any promoter of interest, including a cytomegalovirus promoter. Optionally, an enhancer, such as a cytomegalovirus enhancer, is included in the construct. The cassettes can be suitable for replication and integration in either prokaryotes or eukaryotes. Typical expression cassettes contain specific sequences useful for regulation of the expression of the DNA encoding the protein. For example, the expression cassettes can include appropriate promoters, enhancers, transcription and translation terminators, initiation sequences, a start codon (i.e., ATG) in front of a protein-encoding gene, splicing signals for introns, sequences for the maintenance of the correct reading frame of that gene to permit proper translation of mRNA, and stop codons. The vector can encode a selectable marker, such as a marker encoding drug resistance (for example, ampicillin or tetracycline resistance).

To obtain high level expression of a cloned gene, expression cassettes can include, for example, a strong promoter to direct transcription, a ribosome binding site for translational initiation (e.g., internal ribosomal binding sequences), and a transcription/translation terminator. For E. coli, this can include a promoter such as the T7, trp, lac, or lambda promoters, a ribosome binding site, and a transcription termination signal. For eukaryotic cells, the control sequences can include a promoter and/or an enhancer derived from, for example, an immunoglobulin gene, HTLV, SV40 or cytomegalovirus, and a polyadenylation sequence, and can further include splice donor and/or acceptor sequences (for example, CMV and/or HTLV splice acceptor and donor sequences). The cassettes can be transferred into the chosen host cell by any suitable method such as transformation or electroporation for E. coli and calcium phosphate treatment, electroporation or lipofection for mammalian cells. Cells transformed by the cassettes can be selected by resistance to antibiotics conferred by genes contained in the cassettes, such as the amp, gpt, neo and hyg genes.

Modifications can be made to a nucleic acid encoding an antibody described herein without diminishing its biological activity. Some modifications can be made to facilitate the cloning, expression, or incorporation of the antibody into a fusion protein. Such modifications include, for example, termination codons, sequences to create conveniently located restriction sites, and sequences to add a methionine at the amino terminus to provide an initiation site, or additional amino acids (such as poly His) to aid in purification steps.

Once expressed, the antibodies, fusion proteins, and conjugates can be purified according to standard procedures, including ammonium sulfate precipitation, affinity columns, column chromatography, and the like (see, generally, Simpson et al. (Eds.), Basic methods in Protein Purification and Analysis: A Laboratory Manual, New York: Cold Spring Harbor Laboratory Press, 2009). The antibodies, fusion proteins, and conjugates need not be 100% pure. Once purified, partially or to homogeneity as desired, if to be used prophylactically, the antibodies should be substantially free of endotoxin.

Methods for expression of antibodies, fusion proteins, and conjugates, and/or refolding to an appropriate active form, from mammalian cells, and bacteria such as E. coli have been described and are applicable to the antibodies disclosed herein. See, e.g., Greenfield (Ed.), Antibodies: A Laboratory Manual, 2^{nd} ed. New York: Cold Spring Harbor Laboratory Press, 2014, Simpson et al. (Eds.), Basic methods in Protein Purification and Analysis: A Laboratory Manual, New York: Cold Spring Harbor Laboratory Press, 2009, and Ward et al., Nature 341(6242):544-546, 1989.

### X. Compositions

Compositions are provided that include one or more of the disclosed polypeptides (such as single-domain monoclonal antibodies) that bind (for example specifically bind) coronavirus spike protein S2 subunit in a carrier. Compositions comprising fusion proteins (such as V_{H}H-Fc or V_{NAR}-Fc fusion proteins, or nanobodies in IgG, IgA or IgM format), ADCs, CARs (and immune cells expressing CARs), multi-specific (such as bispecific or trispecific) antibodies, antibody-nanoparticle conjugates, immunoliposomes and immunoconjugates are also provided, as are nucleic acid molecule and vectors encoding the antibodies or antibody conjugates. The compositions can be prepared in unit dosage form for administration to a subject. The amount and timing of administration are at the discretion of the treating clinician to achieve the desired outcome. The polypeptide, antibody, fusion protein, ADC, CAR, CAR-expressing cell, multi-specific antibody, antibody-nanoparticle conjugate, immunoliposome or immunoconjugate can be formulated for systemic or local administration. In one example, the composition is formulated for inhalation administration. In one example, the composition is formulated for intravenous administration. In one example, the composition is lyophilized. In another example, the composition is formulated for oral administration. For example, a composition can include yeast or bacteria formulated to express a disclosed polypeptide (such as a single-domain monoclonal antibody).

In some aspects, the composition includes more than one S2 subunit-specific single-domain monoclonal antibody disclosed herein, such as 2, 3, 4 or 5 different antibodies.

The compositions for administration can include a solution of the antibody, fusion protein, ADC, CAR, CAR-expressing cell (such as a T cell, B cell, NK cell, macrophage or iPSC), multi-specific (such as bispecific or trispecific) antibody, antibody-nanoparticle conjugate, immunoliposome or immunoconjugate in a pharmaceutically acceptable carrier, such as an aqueous carrier. A variety of aqueous carriers can be used, for example, buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of antibody, conjugate, nucleic acid/vector or immune cell in these formulations can vary, and can be selected based on fluid volumes, viscosities, body weight and the like in accordance with the particular mode of administration selected and the subject's needs.

An exemplary pharmaceutical composition for intravenous administration includes about 0.1 to 10 mg of antibody (or fusion protein, ADC, CAR, multi-specific antibody, antibody-nanoparticle conjugate, or immunoconjugate) per subject per day. Dosages from 0.1 up to about 100 mg per subject per day may be used, particularly if the agent is administered to a secluded site and not into the circulatory or lymph system, such as into a body cavity or into a lumen of an organ. In some aspects, the composition can be a liquid formulation including one or more antibodies in a concentration range from about 0.1 mg/ml to about 20 mg/ml, or from about 0.5 mg/ml to about 20 mg/ml, or from about 1 mg/ml to about 20 mg/ml, or from about 0.1 mg/ml to about 10 mg/ml, or from about 0.5 mg/ml to about 10 mg/ml, or from about 1 mg/ml to about 10 mg/ml. Actual methods for preparing administrable compositions will be known or apparent to those skilled in the art and are described in more detail in such publications as Remington: The Science and Practice of Pharmacy, The University of the Sciences in Philadelphia, Editor, Lippincott, Williams, & Wilkins, Philadelphia, PA, 21^{st} Edition (2005).

The monoclonal antibodies disclosed herein can also be administered by other routes, including via inhalation or orally, such as by oral administration of yeast or bacteria (e.g., Lactococcus lactis) engineered to express a disclosed antibody (see, e.g., Vandenbroucke et al., Mucosal Immunol 3(1):49-56, 2010).

Antibodies (or antibody conjugates, or nucleic acid molecules encoding such molecules) may be provided in lyophilized form and rehydrated with sterile water before administration, although they are also provided in sterile solutions of known concentration. The antibody solution can be added to an infusion bag containing 0.9% sodium chloride, USP, and in some cases administered at a dosage of from 0.5 to 15 mg/kg of body weight. Considerable experience is available in the art in the administration of antibody drugs, which have been marketed in the U.S. since the approval of RITUXAN™ in 1997. Antibodies, fusion proteins, ADCs, CARs (or CAR-expressing cells), multi-specific (such as bispecific or trispecific) antibodies, antibody-nanoparticle conjugates, immunoliposomes or immunoconjugates can be administered by slow infusion, rather than in an intravenous push or bolus. In one example, a higher loading dose is administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30-minute period if the previous dose was well tolerated.

Controlled release parenteral formulations can be made as implants, oily injections, or as particulate systems. For a broad overview of protein delivery systems see, Banga, A. J., Therapeutic Peptides and Proteins: Formulation, Processing, and Delivery Systems, Technomic Publishing Company, Inc., Lancaster, PA, (1995). Particulate systems include, for example, microspheres, microparticles, microcapsules, nanocapsules, nanospheres, and nanoparticles. Microcapsules contain the therapeutic protein, such as a cytotoxin or a drug, as a central core. In microspheres the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 m so that only nanoparticles are administered intravenously. Microparticles are typically around 100 μm in diameter and are administered subcutaneously or intramuscularly. See, for example, Kreuter, J., Colloidal Drug Delivery Systems, J. Kreuter, ed., Marcel Dekker, Inc., New York, NY, pp. 219-342 (1994); and Tice & Tabibi, Treatise on Controlled Drug Delivery, A. Kydonieus, ed., Marcel Dekker, Inc. New York, NY, pp. 315-339, (1992).

Polymers can be used for ion-controlled release of the antibody-based compositions disclosed herein. Various degradable and nondegradable polymeric matrices for use in controlled drug delivery are known (Langer, Accounts Chem. Res. 26:537-542, 1993). For example, the block copolymer, polaxamer 407, exists as a viscous yet mobile liquid at low temperatures but forms a semisolid gel at body temperature. It is an effective vehicle for formulation and sustained delivery of recombinant interleukin-2 and urease (Johnston et al., Pharm. Res. 9:425-434, 1992; and Pec et al., J. Parent. Sci. Tech. 44(2):58-65, 1990). Alternatively, hydroxyapatite has been used as a microcarrier for controlled release of proteins (Ijntema et al., Int. J. Pharm. 112:215-224, 1994). In yet another aspect, liposomes are used for controlled release as well as drug targeting of the lipid-capsulated drug (Betageri et al., Liposome Drug Delivery Systems, Technomic Publishing Co., Inc., Lancaster, PA (1993)). Numerous additional systems for controlled delivery of therapeutic proteins are known (see U.S. Pat. Nos. 5,055,303; 5,188,837; 4,235,871; 4,501,728; 4,837,028; 4,957,735; 5,019,369; 5,055,303; 5,514,670; 5,413,797; 5,268,164; 5,004,697; 4,902,505; 5,506,206; 5,271,961; 5,254,342 and 5,534,496).

### XI. Therapeutic Methods

Methods are disclosed herein for the reduction or inhibition of a coronavirus infection in a subject, such as a SARS-CoV and/or a SARS-CoV-2 (including variants thereof) infection. In some examples, the subject has COVID-19. The methods include administering to the subject a therapeutically effective amount (that is, an amount effective to reduce or inhibit the infection in the subject) of a disclosed antibody, fusion protein, ADC, CAR, CAR-expressing cell (such as a T cell, B cell, NK cell, macrophage or iPSC), multi-specific (such as bispecific or trispecific) antibody, antibody-nanoparticle conjugate, immunoliposome or immunoconjugate, or a nucleic acid encoding such an antibody or antibody conjugate, to a subject at risk of a coronavirus infection or having a coronavirus infection. The methods can be used pre-exposure or post-exposure.

The infection does not need to be completely eliminated or inhibited for the method to be effective. For example, the method can decrease the infection by a desired amount, for example by at least 10%, at least 20%, at least 30%, at least 40%, at least 50%, at least 60%, at least 70%, at least 80%, at least 90%, at least 95%, at least 98%, at least 99%, or even 100% (elimination or prevention of detectable coronavirus infection) as compared to the coronavirus infection in the absence of the treatment. In some aspects, the subject can also be treated with an effective amount of an additional agent, such as an anti-viral agent (such as remdesivir).

In some aspects, administration of a therapeutically effective amount of a disclosed antibody, fusion protein, ADC, CAR, CAR-expressing cell (such as a T cell, B cell, NK cell, macrophage or iPSC), multi-specific (such as bispecific or trispecific) antibody, antibody-nanoparticle conjugate, immunoliposome or immunoconjugate, or nucleic acid molecule or vector encoding such molecules, inhibits the establishment of an infection and/or subsequent disease progression in a subject, which can encompass any statistically significant reduction in activity (for example, virus replication) or symptoms of the coronavirus infection in the subject (such as fever or cough).

Methods are disclosed herein for the reduction or inhibition of coronavirus replication in a subject, such as inhibition of SARS-CoV, SARS-CoV-2, MERS-CoV, HKU1-CoV, OC43-CoV, 229E-CoV, or NL63-CoV replication. In specific examples, the coronavirus is SARS-CoV-2 or SARS-CoV. In specific examples, the method can reduce or inhibit replication of more than one type of SARS-CoV-2, such as native SARS-CoV-2 and one or more variants thereof, such as one or more of: alpha (B.1.1.7 and Q lineages); beta (B.1.351 and descendent lineages); delta (B.1.617.2 and AY lineages); gamma (P.1 and descendent lineages); epsilon (B.1.427 and B.1.429); eta (B.1.525); iota (B.1.526); kappa (B.1.617.1); 1.617.3; mu (B.1.621, B.1.621.1); zeta (P.2); and omicron (B.1.1.529, BA.1, BA.1.1, BA.2, BA.3, BA.4 and BA.5). In specific examples, the method can reduce or inhibit replication of SARS-CoV-2 and one, two three, four, five or all of SARS-CoV, MERS-CoV, HKU1-CoV, OC43-CoV, 229E-CoV, or NL63-CoV. The methods include administering to the subject a therapeutically effective amount (that is, an amount effective to inhibit replication in the subject) of a disclosed antibody, antigen binding fragment, or a nucleic acid encoding such an antibody or antigen binding fragment, to a subject at risk of a coronavirus infection or having a coronavirus infection. In some examples, replication of SARS-CoV, SARS-CoV-2 (native and/or variants thereof), MERS-CoV, HKU1-CoV, OC43-CoV, 229E-CoV, or NL63-CoV is inhibited by at least 10%, at least 20%, at least 30%, at least 40%, at least 50%, at least 60%, at least 70%, at least 80%, at least 90%, at least 95%, at least 98%, at least 99%, or even 100% (elimination of detectable coronavirus infection). The methods can be used pre-exposure or post-exposure.

Methods are disclosed for treating a coronavirus infection in a subject. Methods are also disclosed for preventing a coronavirus infection in a subject. These methods include administering one or more of the disclosed antibodies, fusion proteins, ADC, CAR, CAR-expressing cells (such as a T cells, B cells, NK cells, macrophages or iPSCs), multi-specific (such as bispecific or trispecific) antibody, antibody-nanoparticle conjugate, immunoliposome or immunoconjugate, or nucleic acid molecule or vector encoding such molecules, or a composition including such molecules, as disclosed herein. In some examples, the coronavirus is SARS-CoV, SARS-CoV-2, MERS-CoV, HKU1-CoV, OC43-CoV, 229E-CoV, or NL63-CoV. In specific examples, the coronavirus is SARS-CoV-2 or SARS-CoV. In specific examples, the coronavirus is SARS-CoV-2 or a variant thereof, such as: alpha (B.1.1.7 and Q lineages); beta (B.1.351 and descendent lineages); delta (B.1.617.2 and AY lineages); gamma (P.1 and descendent lineages); epsilon (B.1.427 and B.1.429); eta (B.1.525); iota (B.1.526); kappa (B.1.617.1); 1.617.3; mu (B.1.621, B.1.621.1); zeta (P.2); or omicron (B.1.1.529, BA.1, BA.1.1, BA.2, BA.3, BA.4 and BA.5). In some aspects, the subject is a human subject. In other aspects, the subject is a non-human animal subject, such as a non-human primate, cat, dog, bank vole, ferret, fruit bat, hamster, mink, otter, pig, rabbit, raccoon dog, tree shrew or deer.

Antibodies can be administered, for example, by intravenous infusion. Doses of the antibody or conjugate thereof can vary, but generally range between about 0.5 mg/kg to about 50 mg/kg, such as a dose of about 1 mg/kg, about 5 mg/kg, about 10 mg/kg, about 20 mg/kg, about 30 mg/kg, about 40 mg/kg, or about 50 mg/kg. In some aspects, the dose of the antibody or conjugate can be from about 0.5 mg/kg to about 5 mg/kg, such as a dose of about 1 mg/kg, about 2 mg/kg, about 3 mg/kg, about 4 mg/kg or about 5 mg/kg. The antibody or conjugate is administered according to a dosing schedule determined by a medical practitioner. In some examples, the antibody or conjugate is administered weekly, every two weeks, every three weeks or every four weeks.

In some aspects, a subject is administered DNA or RNA encoding a disclosed antibody to provide in vivo antibody production, for example using the cellular machinery of the subject. Any suitable method of nucleic acid administration may be used; non-limiting examples are provided in U.S. Pat. Nos. 5,643,578, 5,593,972 and 5,817,637. U.S. Pat. No. 5,880,103 describes several methods of delivery of nucleic acids encoding proteins to an organism. One approach to administration of nucleic acids is direct administration with plasmid DNA, such as with a mammalian expression plasmid. The nucleotide sequence encoding the disclosed antibody, or antigen binding fragments thereof, can be placed under the control of a promoter to increase expression. The methods include liposomal delivery of the nucleic acids. Such methods can be applied to the production of an antibody, or antigen binding fragments thereof.

In several aspects, a subject (such as a human subject at risk of a coronavirus infection or having a coronavirus infection) is administered an effective amount of a viral vector that includes one or more nucleic acid molecules encoding a disclosed antibody. The viral vector is designed for expression of the nucleic acid molecules encoding a disclosed antibody, and administration of the effective amount of the viral vector to the subject leads to expression of an effective amount of the antibody in the subject. Non-limiting examples of viral vectors that can be used to express a disclosed antibody or antigen binding fragment in a subject include those provided in Johnson et al., Nat. Med., 15(8):901-906, 2009 and Gardner et al., Nature, 519(7541):87-91, 2015.

In one aspect, a nucleic acid encoding a disclosed antibody, or conjugate thereof, is introduced directly into tissue. For example, the nucleic acid can be loaded onto gold microspheres by standard methods and introduced into the skin by a device such as Bio-Rad's HELIOS™ Gene Gun. The nucleic acids can be “naked,” consisting of plasmids under control of a strong promoter.

Typically, the DNA is injected into muscle, although it can also be injected directly into other sites. Dosages for injection are usually around 0.5 μg/kg to about 50 mg/kg, and typically are about 0.005 mg/kg to about 5 mg/kg (see, e.g., U.S. Pat. No. 5,589,466).

Single or multiple administrations of a composition including a disclosed antibody or antibody conjugate, or nucleic acid molecule encoding such molecules, can be administered depending on the dosage and frequency as required and tolerated by the patient. The dosage can be administered once, but may be applied periodically until either a desired result is achieved or until side effects warrant discontinuation of therapy. Generally, the dose is sufficient to inhibit a coronavirus infection without producing unacceptable toxicity to the patient.

Data obtained from cell culture assays and animal studies can be used to formulate a range of dosage for use in humans. The dosage normally lies within a range of circulating concentrations that include the ED₅₀, with little or minimal toxicity. The dosage can vary within this range depending upon the dosage form employed and the route of administration utilized.

The coronavirus S2 subunit-specific antibody, antibody conjugate, or nucleic acid molecule encoding such molecules, or a composition including such molecules, can be administered to subjects in various ways, including local and systemic administration, such as, e.g., by injection subcutaneously, intravenously, intra-arterially, intraperitoneally, intramuscularly, intradermally, or intrathecally. In some aspects, the composition is administered by inhalation, such as by using an inhaler. In one aspect, the antibody, antigen binding fragment, or nucleic acid molecule encoding such molecules, or a composition including such molecules, is administered by a single subcutaneous, intravenous, intra-arterial, intraperitoneal, intramuscular, intradermal or intrathecal injection once a day. The antibody, antigen binding fragment, bispecific antibody, conjugate, or nucleic acid molecule encoding such molecules, or a composition including such molecules, can also be administered by direct injection at or near the site of disease. A further method of administration is by osmotic pump (e.g., an Alzet pump) or mini-pump (e.g., an Alzet mini-osmotic pump), which allows for controlled, continuous and/or slow-release delivery of the antibody, antibody conjugate, or nucleic acid molecule encoding such molecules, or a composition including such molecules, over a pre-determined period. The osmotic pump or mini-pump can be implanted subcutaneously, or near a target site.

In one example, an S2 subunit-specific polypeptide (such as antibody) provided herein is conjugated to IR700, and photoimmunotherapy is used to treat a coronavirus infection. For example, such a method can include administering to the subject with a coronavirus infection a therapeutically effective amount of one or more S2 subunit-specific antibody-IR700 conjugates, wherein the S protein-specific antibody specifically binds to S protein on infected cells. Following administration of the conjugate, irradiation is performed at a wavelength of 660 to 740 nm (such as 660 to 710 nm, for example, 680 nm) and at a dose of at least 1 J cm 2, thereby treating the coronavirus infection in the subject. In some examples, the coronavirus infection is irradiated at a wavelength of 660 to 740 nm (such as 660 to 710 nm, for example, 680 nm) at a dose of at least 1 J cm⁻² (such as at least 1 J cm⁻², at least 4 J cm-2, at least 10 J cm-2, at least 50 J cm², or at least 100 J cm⁻²) thereby treating the coronavirus infection in the subject. In some examples, multiple rounds of treatment are performed, such as 2, 3, 4, 5, 6, 7, 8, 9 or 10 treatment cycles. In particular examples, a therapeutically effective dose of an S2 subunit-specific antibody-IR700 conjugate is at least 0.5 milligram per 60 kilogram (mg/kg), at least 5 mg/60 kg, at least 10 mg/60 kg, at least 20 mg/60 kg, at least 30 mg/60 kg, at least 50 mg/60 kg, for example 0.5 to 50 mg/60 kg, such as a dose of 1 mg/60 kg, 2 mg/60 kg, 5 mg/60 kg, 20 mg/60 kg, or 50 mg/60 kg, for example when administered intravenously. In another example, a therapeutically effective dose of an S2 subunit-specific antibody-IR700 conjugate is at least 10 μg/kg, such as at least 100 μg/kg, at least 500 μg/kg, or at least 500 μg/kg, for example 10 μg/kg to 1000 μg/kg, such as a dose of 100 μg/kg, 250 μg/kg, about 500 μg/kg, 750 μg/kg, or 1000 μg/kg, for example when administered i.p. In one example, a therapeutically effective dose of an S2 subunit-specific antibody-IR700 conjugate is at least 1 μg/ml, such as at least 500 μg/ml, such as between 20 μg/ml to 100 μg/ml, such as 10 μg/ml, 20 μg/ml, 30 μg/ml, 40 μg/ml, 50 μg/ml, 60 μg/ml, 70 μg/ml, 80 μg/ml, 90 μg/ml or 100 μg/ml administered in a topical solution.

In some aspects, the method of treating a coronavirus infection in a subject further includes administration of one or more additional agents to the subject. Additional exemplary agents that can be used include, but are not limited to, anti-viral agents such as remdesivir, galidesivir, favipiravir, baricitinib, lopinavir/ritonavir, hydroxychloroquine, dexamethasone, molnupiravir (Merck), arbidol, zinc ions, and interferon beta-Ib, or their combinations.

Kits are provided for treating or preventing coronavirus infection, such as SARS-CoV-2 infection. Kits for treating or preventing a coronavirus infection include a monoclonal antibody that specifically binds coronavirus S2 subunit, such as one or more of the nanobodies disclosed herein. In some examples, such a kit includes a means for administering the antibody in the kit to a subject, such as a syringe, needle and/or nebulizer. In some examples, such a kit includes additional therapeutic agents, such as one or more additional anti-viral agents, such as remdesivir, galidesivir, favipiravir, baricitinib, lopinavir/ritonavir, hydroxychloroquine, dexamethasone, molnupiravir (Merck), arbidol, zinc ions, and/or interferon beta-lb.

### XII. Methods for Diagnosis and Detection

Methods are also provided for the detection of the presence of a coronavirus spike protein in vitro or in vivo. For example, the disclosed nanobodies can be used for in vivo imaging to detect a coronavirus infection. To use the disclosed antibodies as diagnostic reagents in vivo, the antibodies are labelled directly or indirectly with a detectable moiety, such as a radioisotope, fluorescent label, or positron emitting radionuclides. As one example, the nanobodies disclosed herein can be conjugated to a positron emitting radionuclide for use in positron emission tomography (PET); this diagnostic process is often referred to as immunoPET. While full length antibodies can make good immunoPET agents, their biological half-life necessitates waiting several days prior to imaging, which increases associated non-target radiation doses. Smaller, single domain antibodies/nanobodies, such as those disclosed herein, have biological half-lives amenable to same day imaging.

In some examples, the presence of a coronavirus spike protein is detected in a biological sample from a subject and can be used to identify a subject with a coronavirus infection. In some examples, the coronavirus is SARS-CoV, SARS-CoV-2, MERS-CoV, HKU1-CoV, OC43-CoV, 229E-CoV, or NL63-CoV. In specific examples, the coronavirus is SARS-CoV-2 or SARS-CoV. The sample can be any sample, including, but not limited to, sputum, saliva, mucus, nasal wash, nasopharyngeal samples, oropharyngeal samples, peripheral blood, tissue, cells, urine, tissue biopsy, fine needle aspirate, surgical specimen, feces, cerebral spinal fluid (CSF), and bronchoalveolar lavage (BAL) fluid. Biological samples also include sections of tissues, for example, frozen sections taken for histological purposes. The method of detection can include contacting a cell or sample, with an antibody or antibody conjugate (e.g., a conjugate including a detectable marker) that specifically binds to a coronavirus spike protein, under conditions sufficient to form an immune complex, and detecting the immune complex (e.g., by detecting a detectable marker conjugated to the antibody or antigen binding fragment). In some aspects, the subject from which the sample is obtained is a human subject. In other aspects, the subject from which the sample is obtained is a non-human animal subject, such as a non-human primate, cat, dog, bank vole, ferret, fruit bat, hamster, mink, otter, pig, rabbit, raccoon dog, tree shrew or deer.

In one aspect, the antibody or antigen binding fragment is directly labeled with a detectable marker. In another aspect, the antibody that binds the coronavirus spike protein (the primary antibody) is unlabeled and a secondary antibody or other molecule that can bind the primary antibody is utilized for detection. The secondary antibody can specifically bind the specific species and class of the first antibody. For example, if the first antibody is a human IgG, then the secondary antibody may be an anti-human-IgG. Other molecules that can bind to antibodies include, without limitation, Protein A and Protein G, both of which are available commercially.

Suitable labels for the antibody or secondary antibody include various enzymes, prosthetic groups, fluorescent materials, luminescent materials, magnetic agents and radioactive materials. Non-limiting examples of suitable enzymes include horseradish peroxidase, alkaline phosphatase, beta-galactosidase, or acetylcholinesterase. Non-limiting examples of suitable prosthetic group complexes include streptavidin/biotin and avidin/biotin. Non-limiting examples of suitable fluorescent materials include umbelliferone, fluorescein, fluorescein isothiocyanate, rhodamine, dichlorotriazinylamine fluorescein, dansyl chloride or phycoerythrin. A non-limiting exemplary luminescent material is luminol; a non-limiting exemplary a magnetic agent is gadolinium, and non-limiting exemplary radioactive labels include ¹²⁵I, ¹³¹I, ³⁵S or ³H.

In an alternative aspect, S protein can be assayed in a biological sample by a competition immunoassay utilizing S protein standards labeled with a detectable substance and an unlabeled antibody that specifically binds S protein. In this assay, the biological sample, the labeled S protein standards and the antibody that specifically bind S protein are combined and the amount of labeled S protein standard bound to the unlabeled antibody is determined. The amount of S protein in the biological sample is inversely proportional to the amount of labeled S protein standard bound to the antibody that specifically binds the S2 subunit of a coronavirus S protein.

The immunoassays and methods disclosed herein can be used for a number of purposes. In one aspect, the antibody that specifically binds the S2 subunit of coronavirus S protein may be used to detect the production of S protein in cells in cell culture. In another aspect, the antibody can be used to detect the amount of S protein in a biological sample, such as a sample obtained from a subject having or suspected or having a coronavirus infection.

In one aspect, a kit is provided for detecting coronavirus S protein in a biological sample, such as a nasopharyngeal, oropharyngeal, sputum, saliva, or blood sample. Kits for detecting a coronavirus infection can include a monoclonal antibody that specifically binds coronavirus S protein, such as any of the nanobodies disclosed herein. In a further aspect, the antibody is labeled (for example, with a fluorescent, radioactive, or an enzymatic label). In some examples, such a kit includes a collection device for obtaining the sample, such as collection tubes/vials, syringes, swabs, and the like.

In one aspect, a kit includes instructional materials disclosing means of use of an antibody that binds coronavirus S protein. The instructional materials may be written, in an electronic form or may be visual (such as video files). The kits may also include additional components to facilitate the particular application for which the kit is designed. Thus, for example, the kit may additionally contain means of detecting a label (such as enzyme substrates for enzymatic labels, filter sets to detect fluorescent labels, appropriate secondary labels such as a secondary antibody, or the like). The kits may additionally include buffers and other reagents used for the practice of a particular method.

In one aspect, the diagnostic kit comprises an immunoassay. Although the details of the immunoassays may vary with the particular format employed, the method of detecting S protein in a biological sample generally includes the steps of contacting the biological sample with an antibody which specifically reacts, under immunologically reactive conditions, to coronavirus S protein. The antibody is allowed to specifically bind under immunologically reactive conditions to form an immune complex, and the presence of the immune complex (bound antibody) is detected directly or indirectly.

The antibodies disclosed herein can also be utilized in immunoassays, such as, but not limited to radioimmunoassays (RIAs), ELISA, lateral flow assay (LFA), or immunohistochemical assays. The antibodies can also be used for fluorescence activated cell sorting (FACS), such as for identifying/detecting virus-infected cells. FACS employs a plurality of color channels, low angle and obtuse light-scattering detection channels, and impedance channels, among other more sophisticated levels of detection, to separate or sort cells (see U.S. Pat. No. 5,061,620). Any of the monoclonal antibodies that bind the S2 subunit of spike protein, as disclosed herein, can be used in these assays. Thus, the antibodies can be used in a conventional immunoassay, including, without limitation, ELISA, RIA, LFA, FACS, tissue immunohistochemistry, Western blot or immunoprecipitation. The disclosed nanobodies can also be used in nanotechnology methods, such as microfluidic immunoassays, which can be used to capture SARS-CoV and/or SARS-CoV-2, or exosomes containing these viruses. Suitable samples for use with a microfluidic immunoassay or other nanotechnology method, include but are not limited to, saliva, blood, and fecal samples. Microfluidic immunoassays are described in U.S. Patent Application No. 2017/0370921, 2018/0036727, 2018/0149647, 2018/0031549, 2015/0158026 and 2015/0198593; and in Lin et al., JALA June 2010, pages 254-274; Lin et al., Anal Chem 92: 9454-9458, 2020; and Herr et al., Proc Natl Acad Sci USA 104(13): 5268-5273, 2007). Thus, in some examples, a kit further includes secondary antibodies (which may be labeled), multiwell plates, nitrocellulose, FACS bead standards, and the like.

The following examples are provided to illustrate certain particular features and/or aspects. These examples should not be construed to limit the disclosure to the particular features or aspects described.

### EXAMPLES

### Example 1: Materials and Methods

This example describes the experimental procedures for the studies disclosed in Examples 2-3.

### Construction of Shark and Camel Nanobody Libraries

The shark V_{NAR} phage library was constructed previously (Feng et al., Antib Ther 2, 1-11, 2019) and the camel V_{H}H library construction was performed following the shark phage library protocol with some modifications. Briefly, peripheral blood leukocytes from six adult nurse sharks or six camels were isolated, and cDNA were prepared from those cells. Two-step PCR was performed with a first set of primers covering the region outside the antibody genes using the cDNA as the template, and a second set of primers covering the single domain antibodies using the DNA from the first PCR as the template. The amplified nanobody fragments were assembled with vector backbone pComb3X by over-lapping extension PCR. The V_{NAR} DNA from six shark together was assembled with the phagemid vector for generation of one shark V_{NAR} library with a size of 10¹⁰. Six dromedary camel V_{H}H genes were assembled with the phagemid vector for generation of six individual V_{H}H nanobody libraries with a size of 10¹⁰ for each library. The total diversity of the six V_{H}H libraries used in the present study is greater than 10¹¹.

### Phage Panning and ELISA

Phage panning was performed as previously described. For shark library panning, MAXISORP™ Immuno Tubes (Thermo Scientific) were coated with SARS-CoV-2 S2 (Sino Biological, 40590-V08B) for three rounds of panning. For camel library panning, two approaches were used. In the first approach, MAXISORP™ Immuno Tubes were coated with SARS-CoV-2 S2 for four rounds of panning. In the second approach, SARS-CoV-2 S2 was used for the first round and SARS-CoV-2 spike trimer (40589-V08H4) was used in the second and third rounds of panning.

After phage panning, single colonies were picked for monoclonal ELISA. A MAXISORP™ 96-well plate (Fisher Scientific, 12565136) was coated with SARS-CoV-2 S2 subunit and spike trimer. Phage ELISA was performed accordingly to previously described protocols and results were read using a spectrophotometer (Molecular Devices) at 450 nm.

### Affinity Measurement by Octet

Binding kinetics and competition assays were conducted using the Octet RED96 system (FortéBio). For binding kinetics, the S2 antigen was immobilized onto streptavidin sensor tips. The antigen-coated tips were dipped into PBS 0.1% Tween 20 with 0.1% BSA to stabilize the curve and then dipped into wells containing the single domain antibody for association, followed by dipping into PBS 0.1% Tween20 with 0.1% BSA for dissociation. Raw data was processed using Octet Data Analysis Software 9.0 to determine K_{D} value.

### Flow Cytometry

A431 cells expressing the spike protein from either SARS-CoV-2 or SARS-CoV were constructed for flow cytometry testing of nanobody binding. Either A431-CoV-2-S or A431-CoV-S cell line was stained with the nanobodies followed by anti-FLAG-APC (Jackson ImmunoResearch). Data was collected using Sony Cell Analyzer.

### Construction of Mutant Spike Protein for Virus Variants

Mutant spike proteins for expressing pseudovirus of SARS-CoV-2 variants were constructed into the pVRC8400 plasmid by GenScript. The mutations are listed below. Alpha: H69del, V70del, Y144del, N501Y, A570D, D614G, P681H, T716I, S982A, D1118H. Beta: L18F, D80A, D215G, LAL242-244deletion, R246I, K417N, E484K, N501Y, D614G, A701V. Delta: 157-158deletion, T19R, G142D, E156G, L452R, T478K, D614G, P681R, D950N. Gamma: L18F, T20N, P26S, D138Y, R190S, K417T, E484K, N501Y, D614G, H655Y, T1027I, V1176F. Lambda: G75V, T76I, R246del, S247del, Y248del, L249del, T250del, P251del, G252del, D253N, L452Q, F490S, D614G, T859N. The pseudoviruses were produced following a standard protocol.

### Pseudovirus Neutralization Assays

For the lentivirus-based pseudovirus infection assay, 5000 HEK293T cells expressing human ACE2 (HEK293T-ACE2) were seeded in 96-well plates. An appropriate volume of SARS-CoV-2 (or variant) spike pseudovirus supernatant that can produce a luciferase signal 1000× higher than the baseline was used. Nanobody or hFc fusion proteins were prepared in serial dilutions starting with 50 or 100 μg/ml. The antibodies and viruses were mixed for 45 minutes before adding to HEK 293T-hACE2 cells. After incubation for 72 hours, the luciferase signal was measured by a plate reader. All experiments were performed in triplicate. The infectivity was calculated from the luciferase activity of different groups normalized by cells with virus only group (100%). The graphs were made, and IC₅₀ doses were calculated by GraphPad Prism.

### Example 2: Isolation of Nanobodies Against SARS-CoV-2 S2 Subunit

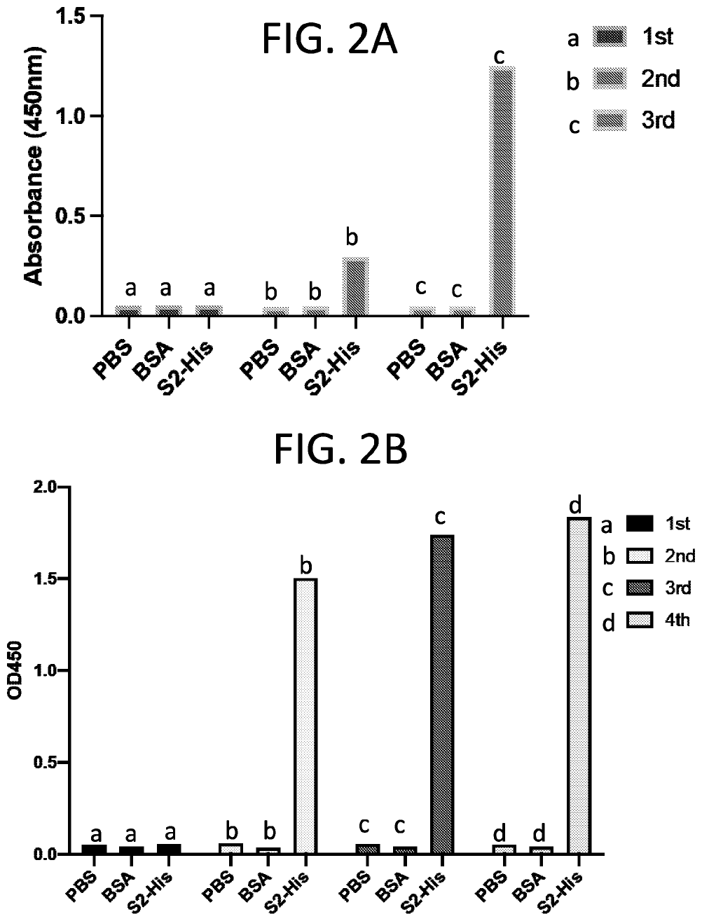

To identify nanobodies against the S2 subunit of SARS-CoV-2, phage panning was performed to screen S2 or the spike trimer protein using either shark V_{NAR} or camel V_{H}H single domain phage display libraries (FIG. 1). Only SARS-CoV-2 S2 was used for shark phage panning, while both SARS-CoV-2 S2 and spike trimer were used for camel phage panning. There was good enrichment of S2-specific phages from polyclonal phage ELISA with phage pools from different rounds of panning (FIGS. 2A-2B). For shark panning, 960 V_{NAR} phage clones were isolated; among them, 327 V_{NAR} clones exhibited a good binding signal for S2. Of the 327 clones, there were 42 unique V_{NAR} sequences. For camel panning, 768 V_{H}H phage clones were isolated; among them, 300 V_{H}H clones had a good binding signal for S2. Of the 300 clones, there were 56 unique V_{H}H sequences.

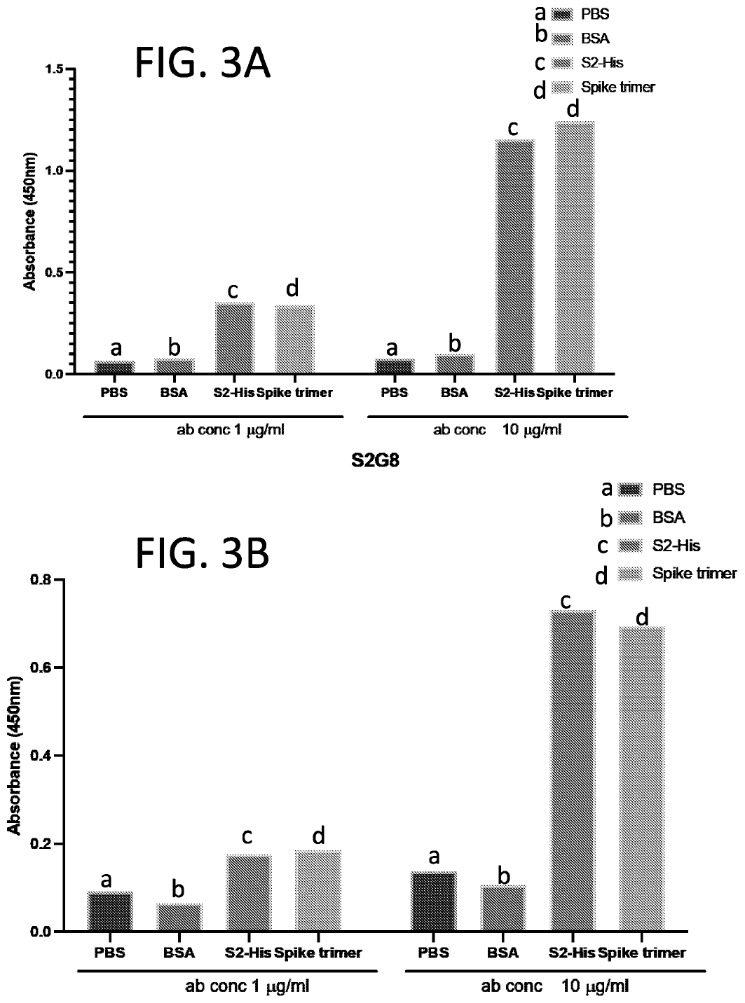

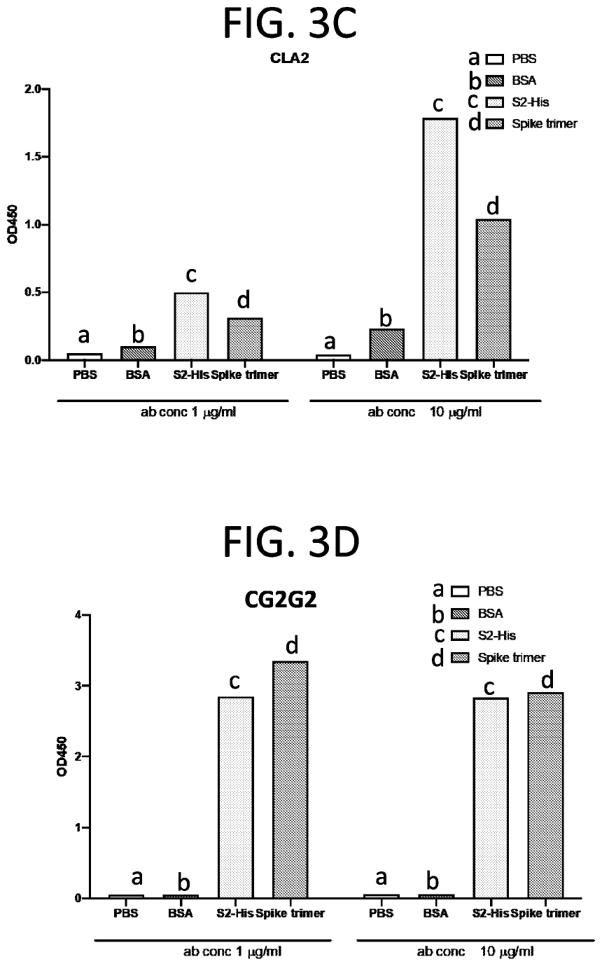

The top 6 V_{NAR} and top 6 V_{H}H single domains that bound both S2 and spike trimer protein of SARS-CoV-2 were selected for further analysis. The sequence alignment of the top 6 V_{NAR} showed that NCI-CoV-S2A9, NCI-CoV-S4A9 and NCI-CoV-S6E1 are type I V_{NAR} containing an even number of cysteines (6, 4 and 4, respectively); and NCI-CoV-S3A10, NCI-CoV-S2G8 and NCI-CoV-S4C12 are type II V_{NAR} containing an odd number of cysteines (5, 3 and 3, respectively). The sequence alignment of the top 6 V_{H}H showed that NCI-CoV-CGH3 is the only V_{H}H containing two canonical cysteines, one N-terminal to CDR1 and the other N-terminal to CDR3, whereas NCI-CoV-CLA2, NCI-CoV-CGF10 and NCI-CoV-CG2D11 have an additional non-canonical cysteine and NCI-CoV-CG2G2 has an additional two non-conical cysteines. A protein ELISA for the top binders was performed to validate the binding of nanobodies to the antigens S2 or spike trimer (FIGS. 3A-3D). NCI-CoV-S2A9 (FIG. 3A), NCI-CoV-S2G8 (FIG. 3B), NCI-CoV-CLA2 (FIG. 3C) and NCI-CoV-CG2G2 (FIG. 3C) showed specific binding to S2 or spike trimer with good binding affinity.

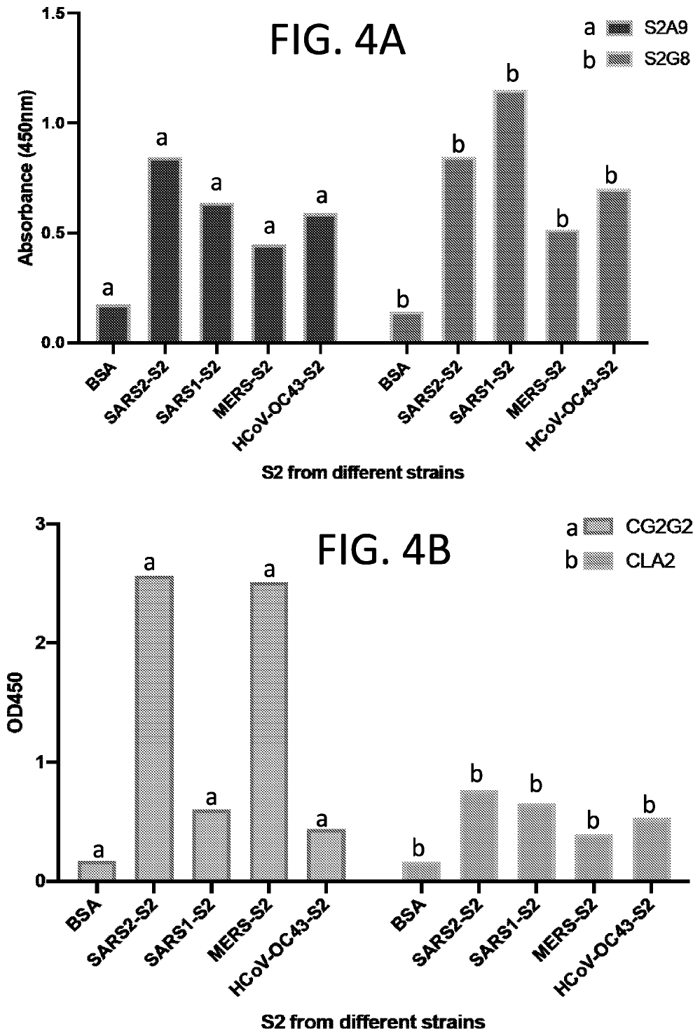

An ELISA with S2 subunits from other coronaviruses was performed to test the cross binding of S2 binders (FIGS. 4A-4B). In addition to SARS-CoV-2, NCI-CoV-S2A9, NCI-CoV-S2G8 and NCI-CoV-CLA2 bound to S2 subunits from SARS-CoV, MERS and HCoV-OC43. NCI-CoV-CG2G2 showed strong cross-binding to MERS S2 and weak binding to SARS-CoV and HCoV-OC43.

### Example 3: Nanobodies Targeting S2 Neutralize SARS-CoV-2 and its Variants

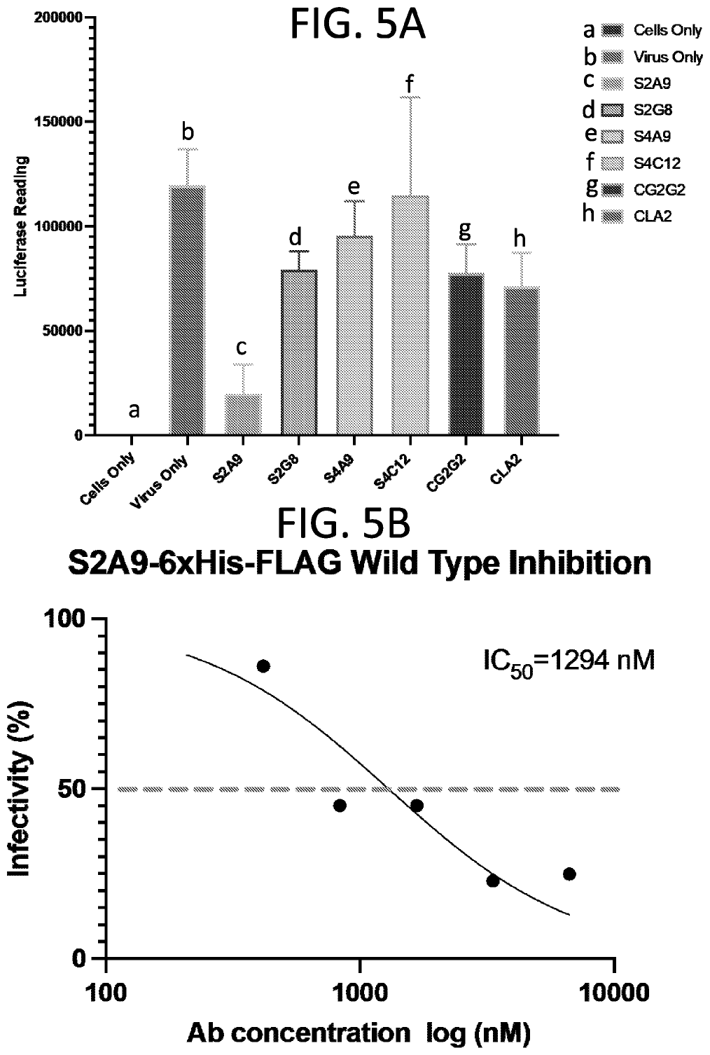

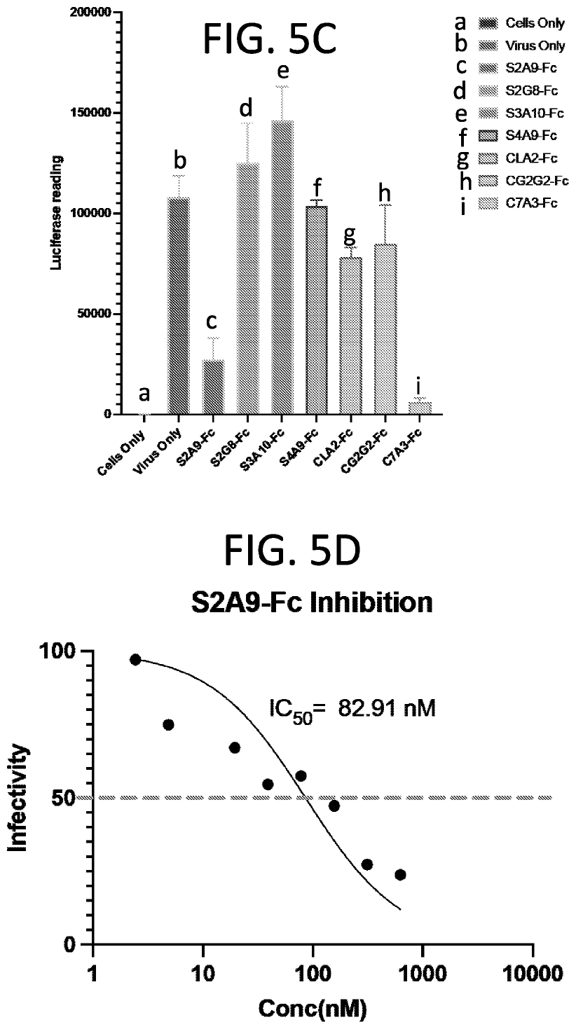

To determine whether the selected nanobodies can neutralize SARS-CoV-2, pseudovirus neutralization assays were performed using the monovalent nanobodies as well as their respective Fc fusions (bivalent). NCI-CoV-S2A9 exhibited the greatest potency to inhibit pseudovirus with an IC₅₀ of 1294 nM (FIGS. 5A-5C). Bivalent NCI-CoV-S2A9-hFc had an IC₅₀ of approximately 83 nM (FIG. 5D), which is an approximately 20-fold increase in neutralization activity compared to monovalent NCI-CoV-S2A9-V_{NAR}.

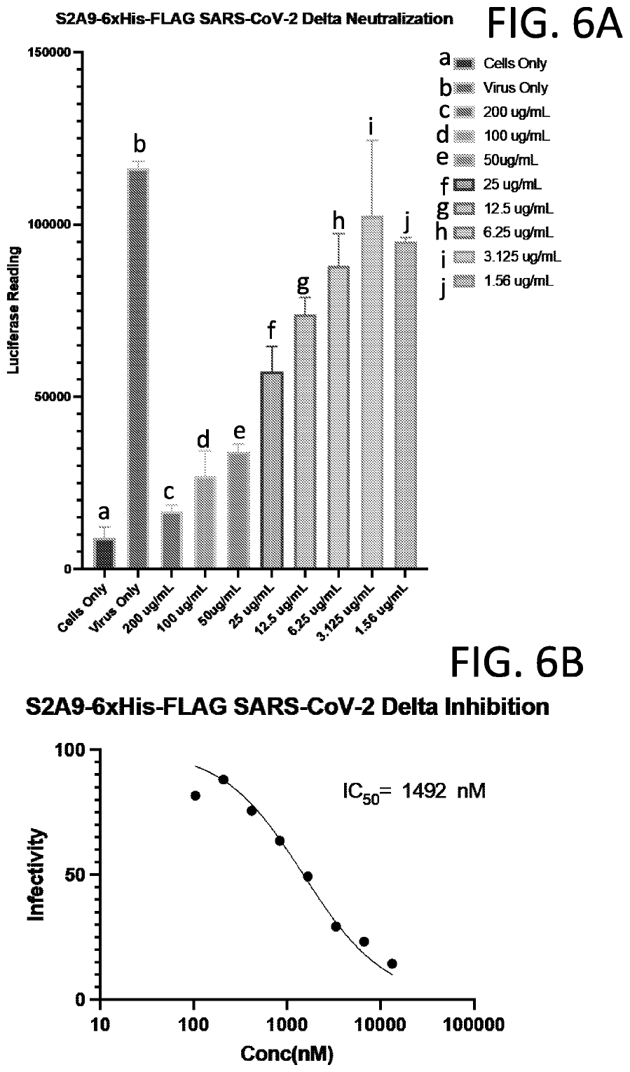

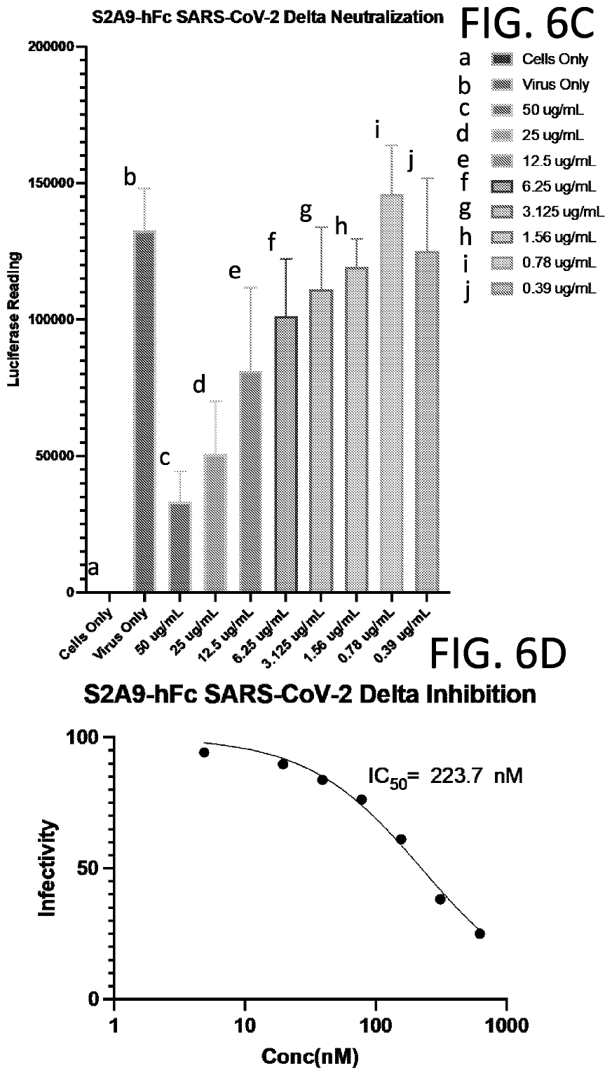

An additional study was conducted to evaluate neutralization of SARS-CoV-2 Delta variant. 293T cells overexpressing hACE2 were plated. Pseudovirus expressing SARS-CoV-2 delta variant spike protein were incubated with the single domain format of S2A9 (S2A9-6×His-FLAG) or bivalent S2A9 with a human Fc tag (S2A9-hFc) at concentrations of 100 μg/ml, 50 μg/ml, 25 μg/ml, 12.5 μg/ml, 6.25 μg/ml, 3.125 g/ml, 1.56 μg/ml or 0.78 μg/ml for S2A9-6×His-FLAG, or at concentrations of 50 μg/ml, 25 μg/ml, 12.5 μg/ml, 6.25 μg/ml, 3.125 μg/ml, 1.56 μg/ml, 0.78 μg/ml or 0.39 μg/ml for S2A9-hFc. The luciferase signal was read by plate reader to calculate the inhibitory activity. IC₅₀ was determined based on inhibitory activity from various antibody concentrations. The results are shown in FIGS. 6A-6D. The IC₅₀ of shark S2A9-6×His-FLAG was 1492 nM and the IC₅₀ of S2A9-hFc was 223.7.

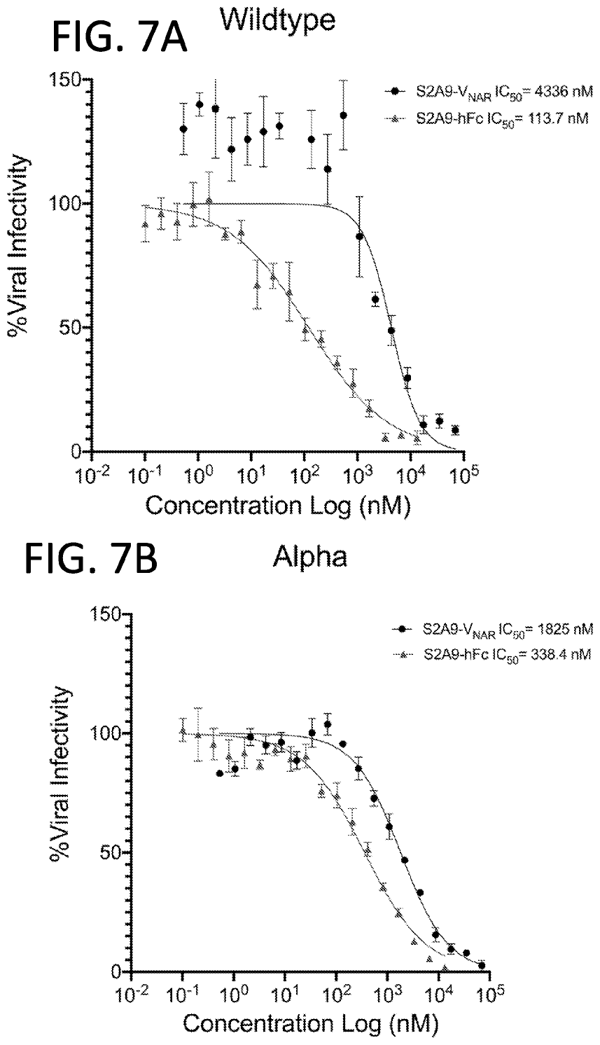

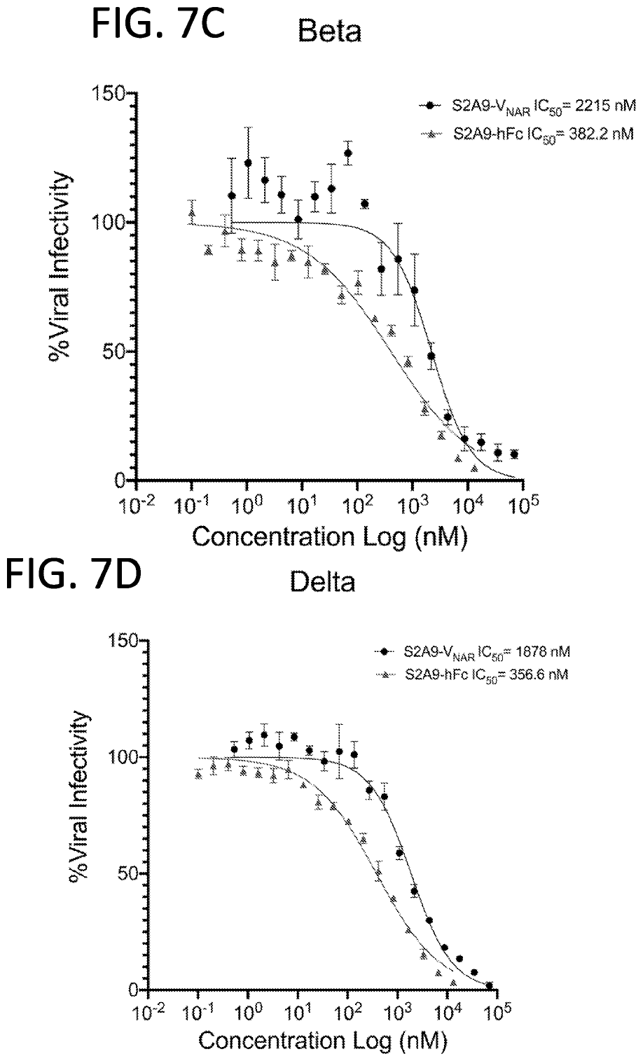

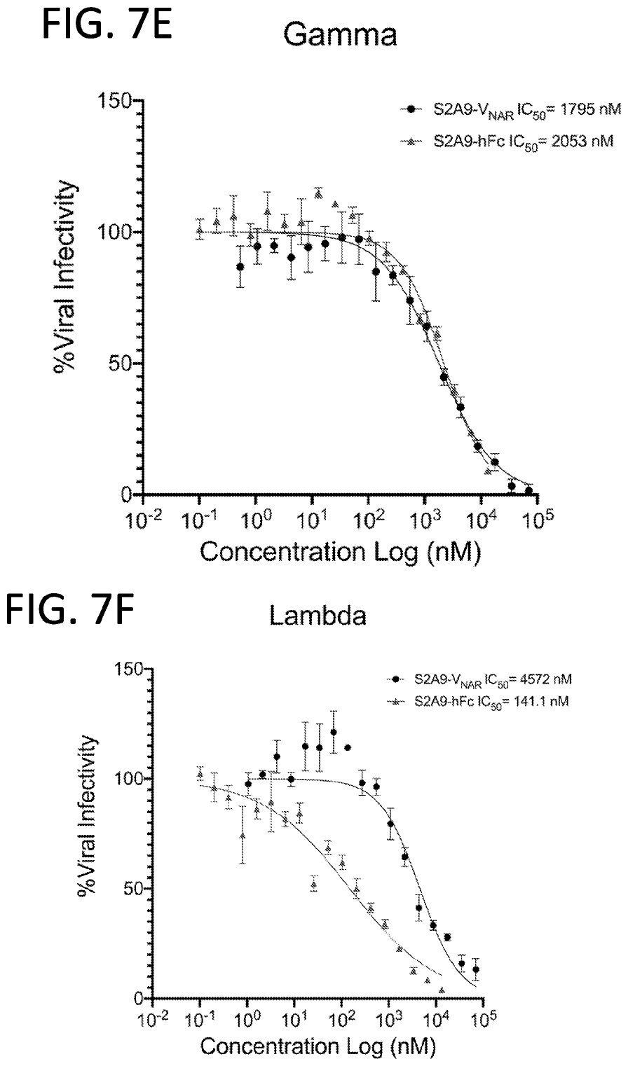

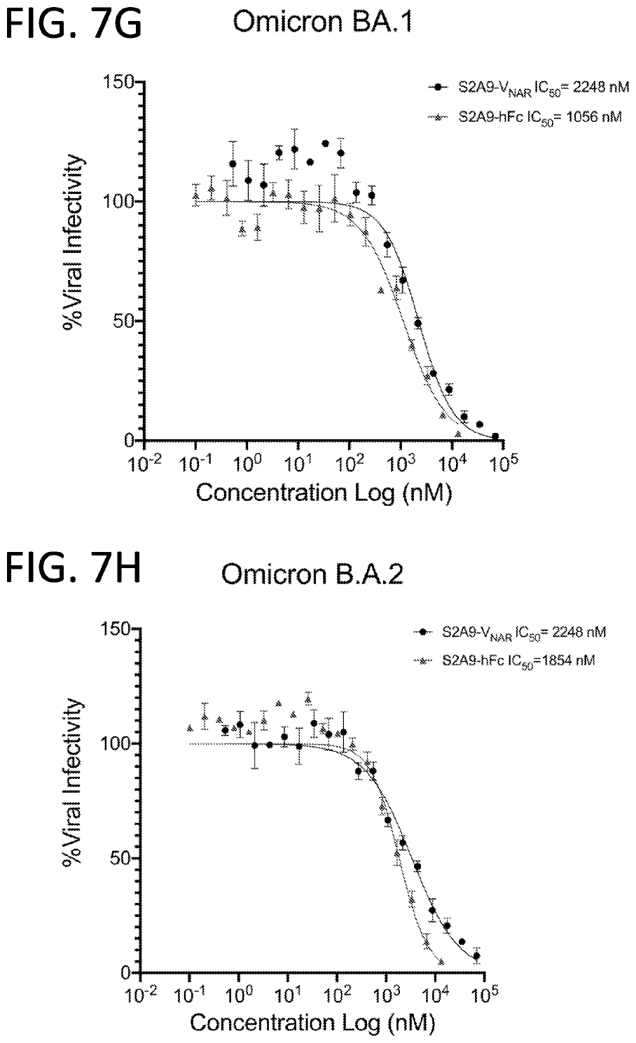

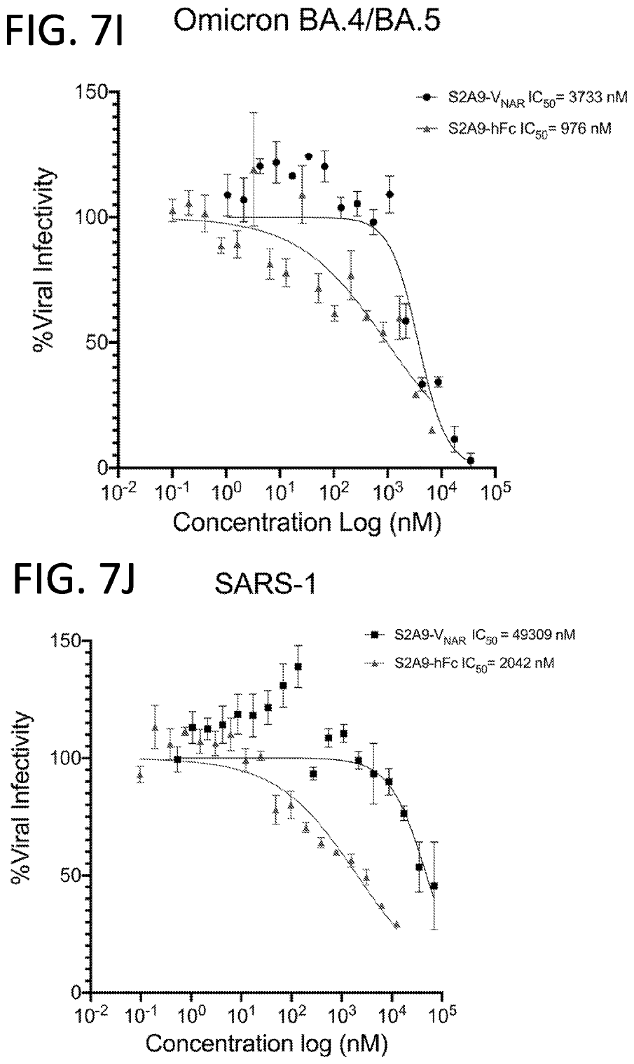

A further study evaluated the ability of S2A9 nanobody and S2A9-Fc to neutralize pseudovirus expressing spike protein from the SARS-CoV-2 Alpha, Beta, Delta, Gamma, Lambda, Omicron BA.1, Omicron BA.2, and Omicron BA.4/BA.5 variants. Pseudovirus expressing spike protein from SARS-CoV-2 (WT) and SARS-CoV-1 were also included. The results are shown in FIGS. 7A-7J and IC₅₀ concentrations are listed in Table 8.

TABLE 8
IC₅₀ of S2A9 and S2A9-hFc for neutralizing
SARS-CoV-2 pseudovirus

| Pseudovirus | S2A9 nanobody IC₅₀ | | S2A9-hFc IC₅₀ | |
|---|---|---|---|---|
| SARS-CoV-2 (WT) | 4336 | nM | 113.7 | nM |
| Alpha variant | 1825 | nM | 338.4 | nM |
| Beta variant | 2215 | nM | 382.2 | nM |
| Delta variant | 1878 | nM | 356.6 | nM |
| Gamma variant | 1795 | nM | 2053 | nM |
| Lambda variant | 4572 | nM | 141.1 | nM |
| Omicron BA.1 variant | 2248 | nM | 1056 | nM |
| Omicron BA.2 variant | 2248 | nM | 1854 | nM |
| Omicron BA.4/BA.5 variant | 3733 | nM | 976 | nM |
| SARS-CoV-1 | 49,309 | nM | 2042 | nM |

Both S2A9 nanobody and S2A9-hFc showed inhibition activity against SARS-CoV-2 and all variants, with S2A9-Fc Fc exhibiting greater inhibition than S2A9 in nanobody form.

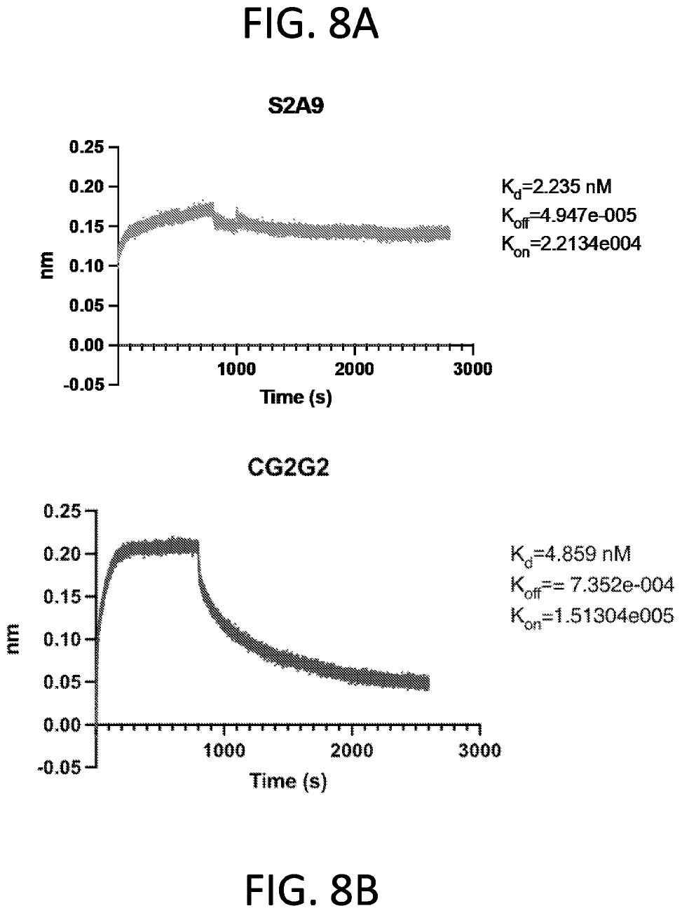

Binding affinity of S2A9 and CG2G2 to the S2 subunit of SARS-CoV-2 was measured by Octet. The results are shown in FIGS. 8A-8B. The K_{d} of S2A9 and CG2G2 binding to S2 was 2.235 nM and 4.859 nM, respectively.

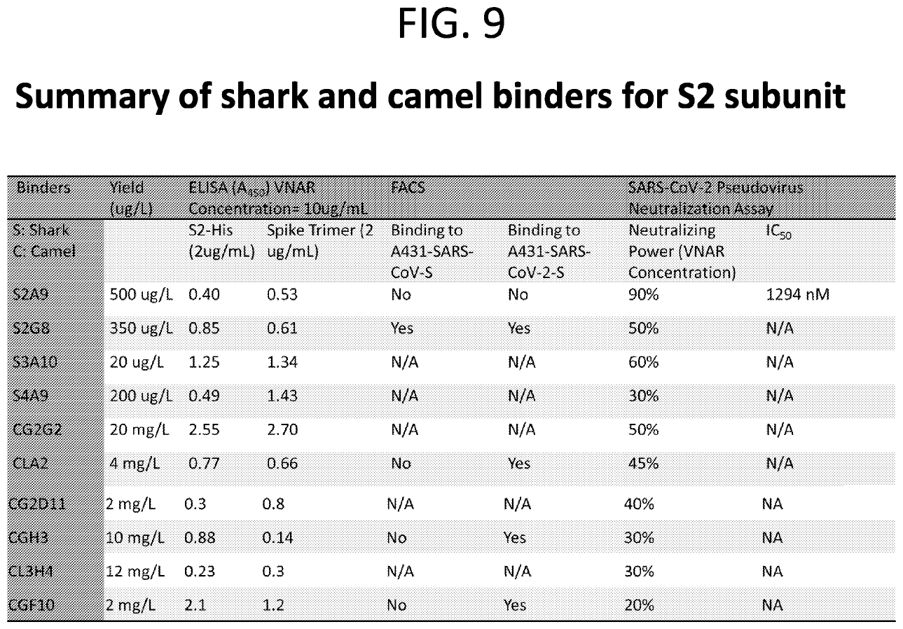

A table summarizing binding characteristics and neutralizing capacity of each nanobody tested is shown in FIG. 9.

In view of the many possible aspects to which the principles of the disclosed subject matter may be applied, it should be recognized that the illustrated aspects are only examples of the disclosure and should not be taken as limiting the scope of the disclosure. Rather, the scope of the disclosure is defined by the following claims. We therefore claim all that comes within the scope and spirit of these claims.

1. A polypeptide that specifically binds the S2 subunit of a severe acute respiratory syndrome coronavirus 2 (SARS-CoV-2) spike protein, wherein the polypeptide comprises:
(i) the complementarity determining region 1 (CDR1) and CDR3 sequences of SEQ ID NO: 1, SEQ ID NO: 2, SEQ ID NO: 3, SEQ ID NO: 4, SEQ ID NO: 5 or SEQ ID NO: 6; or
(ii) the CDR1, CDR2 and CDR3 sequences of SEQ ID NO: 7, SEQ ID NO: 8, SEQ ID NO: 9, SEQ ID NO: 10, SEQ ID NO: 11 or SEQ ID NO: 12.

2. The polypeptide of claim 1(i), wherein the CDR1 and CDR3 sequences respectively comprise:
(a) residues 26-33 and 86-101 of SEQ ID NO: 1;
(b) residues 26-33 and 86-10³ of SEQ ID NO: 2;
(c) residues 26-33 and 86-100 of SEQ ID NO: 3;
(d) residues 26-33 and 86-101 of SEQ ID NO: 4;
(e) residues 26-33 and 86-101 of SEQ ID NO: 5; or
(f) residues 26-33 and 86-97 of SEQ ID NO: 6.

3. The polypeptide of claim 2, further comprising:
a hypervariable (HV) 2 region comprising the amino acid sequence of SEQ ID NO: 25; and/or
a HV4 region comprising the amino acid sequence of SEQ ID NO: 26.

4. (canceled)

5. The polypeptide of claim 2, wherein the amino acid sequence of the polypeptide is at least 90% identical to SEQ ID NO: 1, SEQ ID NO: 2, SEQ ID NO: 3, SEQ ID NO: 4, SEQ ID NO: 5 or SEQ ID NO: 6.

6. The polypeptide of claim 2, wherein the amino acid sequence of the polypeptide comprises or consists of SEQ ID NO: 1, SEQ ID NO: 2, SEQ ID NO: 3, SEQ ID NO: 4, SEQ ID NO: 5 or SEQ ID NO: 6.

7. The polypeptide of claim 1(ii), comprising:
the CDR1, CDR2 and CDR3 sequences of SEQ ID NO: 7, wherein the CDR1, CDR2 and CDR3 sequences respectively comprise: residues 26-32, 50-55 and 95-111 of SEQ ID NO: 7; residues 26-32, 50-55 and 97-111 of SEQ ID NO: 7; or residues 27-35, 47-58 and 96-111 of SEQ ID NO: 7;
the CDR1, CDR2 and CDR3 sequences of SEQ ID NO: 8, wherein the CDR1, CDR2 and CDR3 sequences respectively comprise: residues 26-33, 51-58 and 96-107 of SEQ ID NO: 8; residues 31-35, 50-65 and 98-107 of SEQ ID NO: 8; or residues 26-35, 44-61 and 97-107 of SEQ ID NO: 8;
the CDR1, CDR2 and CDR3 sequences of SEQ ID NO: 9, wherein the CDR1, CDR2 and CDR3 sequences respectively comprise: residues 26-33, 51-58 and 97-113 of SEQ ID NO: 9; residues 31-35, 50-66 and 99-113 of SEQ ID NO: 9; or residues 27-35, 47-60 and 98-113 of SEQ ID NO: 9;
the CDR1, CDR2 and CDR3 sequences of SEQ ID NO: 10, wherein the CDR1, CDR2 and CDR3 sequences respectively comprise: residues 26-33, 51-58 and 97-113 of SEQ ID NO: 10; residues 31-35, 50-66 and 99-114 of SEQ ID NO: 10; or residues 27-35, 47-61 and 97-114 of SEQ ID NO: 10;
the CDR1, CDR2 and CDR3 sequences of SEQ ID NO: 11, wherein the CDR1, CDR2 and CDR3 sequences respectively comprise: residues 26-33, 51-58 and 97-117 of SEQ ID NO: 11; residues 31-35, 50-66 and 99-117 of SEQ ID NO: 11; or residues 26-35, 47-60 and 98-117 of SEQ ID NO: 11; or
the CDR1, CDR2 and CDR3 sequences of SEQ ID NO: 11, wherein the CDR1, CDR2 and CDR3 sequences respectively comprise: residues 26-33, 51-58 and 97-117 of SEQ ID NO: 11; residues 31-35, 50-66 and 99-117 of SEQ ID NO: 11; or residues 26-35, 47-60 and 98-117 of SEQ ID NO: 11.

8-12. (canceled)

13. The polypeptide of claim 7, wherein the amino acid sequence of the polypeptide is at least 90% identical to SEQ ID NO: 7, SEQ ID NO: 8, SEQ ID NO: 9, SEQ ID NO: 10, SEQ ID NO: 11 or SEQ ID NO: 12.

14. The polypeptide of claim 7, wherein the amino acid sequence of the polypeptide comprises or consists of SEQ ID NO: 7, SEQ ID NO: 8, SEQ ID NO: 9, SEQ ID NO: 10, SEQ ID NO: 11 or SEQ ID NO: 12.

15. The polypeptide of claim 1, wherein the polypeptide is a single-domain monoclonal antibody.

16. The polypeptide of claim 15, wherein the single-domain monoclonal antibody is a humanized single-domain monoclonal antibody or a chimeric single-domain antibody.

17. (canceled)

18. A composition comprising at least two different polypeptides of claim 1.

19. The composition of claim 18, further comprising a pharmaceutically acceptable carrier.

20. The composition of claim 18, formulated for administration by inhalation.

21. A fusion protein comprising the polypeptide of claim 1 and a heterologous protein.

22. The fusion protein of claim 21, wherein the heterologous protein is an Fc protein.

23. (canceled)

24. A chimeric antigen receptor (CAR) comprising the polypeptide of claim 1.

25. An isolated cell expressing the CAR of claim 24.

26. The isolated cell of claim 25, wherein the cell is a T cell, a B cell, a natural killer (NK) cell, a macrophage or an induced pluripotent stem cell (iPSC).

27. An immunoconjugate comprising the polypeptide of claim 1 and an effector molecule.

28. The immunoconjugate of claim 27, wherein the effector molecule is a toxin, a detectable label, or a photon absorber.

29-30. (canceled)

31. An antibody-drug conjugate (ADC) comprising a drug conjugated to the polypeptide of claim 1.

32. A multi-specific antibody comprising the polypeptide of claim 1 and at least one additional monoclonal antibody or antigen-binding fragment thereof.

33. The multi-specific antibody of claim 32, which is a bispecific antibody.

34. (canceled)

35. An antibody-nanoparticle conjugate, comprising a nanoparticle conjugated to the polypeptide of claim 1.

36. (canceled)

37. An isolated nucleic acid molecule encoding the polypeptide of claim 1.

38. The isolated nucleic acid molecule of claim 37, comprising the nucleotide sequence of any one of SEQ ID NOs: 13-24, or a degenerate variant thereof.

39. The isolated nucleic acid molecule of claim 37, operably linked to a promoter.

40. A vector comprising the nucleic acid molecule of claim 37.

41. An isolated host cell comprising the vector of claim 40.

42. A composition comprising a pharmaceutically acceptable carrier and the polypeptide of claim 1.

43. A method of detecting a coronavirus in a sample, comprising:
contacting the sample with the polypeptide of claim 1; and
detecting binding of the polypeptide to the sample, thereby detecting the coronavirus in the sample.

44. A method of diagnosing a subject as having a coronavirus infection, comprising:
contacting a sample obtained from the subject with the polypeptide of claim 1; and
detecting binding of the polypeptide to the sample, thereby diagnosing the subject as having a coronavirus infection.

45-47. (canceled)

48. A method of treating a coronavirus infection in a subject, comprising administering to the subject a therapeutically effective amount of the polypeptide of claim 1.

49. The method of claim 48, comprising administering to the subject a therapeutically effective amount of the polypeptide by inhalation.

50. The method of claim 48, wherein the coronavirus is a SARS-CoV-2 or SARS-CoV.

51. The method of claim 48, wherein the subject is a human subject or a non-human animal.

52. (canceled)

53. The method of claim 51, wherein the non-human animal is a non-human primate, cat, dog, bank vole, ferret, fruit bat, hamster, mink, otter, pig, rabbit, raccoon dog, tree shrew or deer.

54. A solid support comprising one or more polypeptides of claim 1.

55. The solid support of claim 54, wherein the solid support comprises a bead, multiwell plate, or nitrocellulose having attached thereto the one or more polypeptides.

56. A method of detecting a coronavirus in a sample, comprising:
contacting the sample with the solid support of claim 54; and
detecting binding of the coronavirus to the one or more polypeptides attached to the solid support, thereby detecting coronavirus in the sample.

57-58. (canceled)


SEQUENCE LISTING (26 sequences)

SEQ ID NO: 1 - protein, 111 aa, synthetic construct
  REGION 1..111: note = Synthetic polypeptide
ARVDQTPRSVTKETGESLTINCVLRDASYALGRTCWYRKKSGSTNEESISKGGRYVETVNSGSKSFSLRINDLTVEDGGTYRCGVSTPWSCDYGPLCFYAACGDGTVVTVN

SEQ ID NO: 2 - protein, 113 aa, synthetic construct
  REGION 1..113: note = Synthetic polypeptide
ARVDQTPQTITKETGESLTINCVLRDSNCPLSNTYWYRKKSGSTNEESISKGGRYVETVNSGSKSFSLRINDLTVEDSGTYRCKVRTHWSICWEMGLEPNYDVYGGGTVVTVN

SEQ ID NO: 3 - protein, 110 aa, synthetic construct
  REGION 1..110: note = Synthetic polypeptide
ARVDQTPRSVTKETGESLTINCVLRDASYALGSTCWYRKKSGSTNEESISKGGRYVETVNSGSKSFSLRINDLTVEDGGTYRCGRIQNTKAVLLPCVYAACGDGTVVTVN

SEQ ID NO: 4 - protein, 111 aa, synthetic construct
  REGION 1..111: note = Synthetic polypeptide
ARVDQTPRSVTKETGESLTINCVLRDASYALGSTCWYRKKSGSTNEESISKGGRYVETVNSGSKSFSLRINDLTVEDGGTYRCGVQSRWGAVPKLCSNYAACGDGTAVTVN

SEQ ID NO: 5 - protein, 111 aa, synthetic construct
  REGION 1..111: note = Synthetic polypeptide
ARVDQTPQTITKETGESLTINCVLRDSNCALSSTYWYRKKSGSTNEESISKGGRYVETVNSGSKSFSLRINDLTVEDSGTYRCKVGCWYGYESSLMNWSRVYGGGTAVTVN

SEQ ID NO: 6 - protein, 107 aa, synthetic construct
  REGION 1..107: note = Synthetic polypeptide
ARVDQTPRSVTKETGESLTINCVLRDASYALGSTCWYRKKSGSTNEESISKGGRYVETVNSGSKSFSLRINDLTVEDSGTYRCKGYTPCMPDWVPDVYGMGTVVTVN

SEQ ID NO: 7 - protein, 122 aa, synthetic construct
  REGION 1..122: note = Synthetic polypeptide
QVQLVESGGGSVQPGGSLRLSCAVSGYTSSSYCFGWFRQAPGKDREGIIAMEPGNTYTAGAVKGRFTISRDNAKNMVYLQMNSLKPDDTAMYYCAASPYPGTWLNPDLFDTWGQGTQVTVSS

SEQ ID NO: 8 - protein, 118 aa, synthetic construct
  REGION 1..118: note = Synthetic polypeptide
AVQLVESGGGSVPPGGSLTLSCTVSDNIFRTYAVGWFRQAPEKRCQWIATLNADESTEYNVAVTGRFTISRDNSGRSVYLQMDNLNSEDTAIYYCASLARKWSCHDSWGRGTQVTVSS

SEQ ID NO: 9 - protein, 124 aa, synthetic construct
  REGION 1..124: note = Synthetic polypeptide
AVQLVESGGGLVQPGGSLRLSCAASGFTFSSYAMGWVRQAPGKGLEWISAINSGGDIRYYADSVKGRFTISRDNAKNTLYLQMNSLKPEDTAVYYCATDDEILSYGLGTSFDYWGQGTQVTVSS

SEQ ID NO: 10 - protein, 125 aa, synthetic construct
  REGION 1..125: note = Synthetic polypeptide
AVQLVESGGGLVQPGGSLRLSCAASGFTFSSYGMHWVRQAPGKGLERVAGINWDGSNSYYTETVKGRFTISRDNAKNTAYLQMNSLKPEDTAMYYCRTLILDSDCDPGSTYRYAWGQGTQVTVSS

SEQ ID NO: 11 - protein, 128 aa, synthetic construct
  REGION 1..128: note = Synthetic polypeptide
AVQLVDSGGGSVQAGGSLRLSCAGSAFTYSSYCMGWFRQAPGKEREAVAAINSGTSSTYYADSVKGRFTVSQDNANNTVYLLMISLKPEDTAIYICAALVAPDYDDYDVAAPDNFDYWGQGTQVTVSS

SEQ ID NO: 12 - protein, 120 aa, synthetic construct
  REGION 1..120: note = Synthetic polypeptide
DVQLVESGGGAVKPGGSLTLTCTVQEGSRNCVGWFRQGSGKDRQAVAMINGNGGTTNYDDRVKGRFQISRDGSLVHLRMNDVKLGDTDIYYCRQGQTYDCWGGTWTDSGWGQGTQVTVSS

SEQ ID NO: 13 - DNA, 333 bp, synthetic construct
  misc_feature 1..333: note = Synthetic nucleic acid
  source 1..333: mol_type = other DNA
gctcgagtggaccaaacaccgagatcagtaacaaaggagacgggcgaatcactgaccatcaactgtgtcctacgagatgcgagctatgcattgggcaggacgtgctggtatcgaaaaaaatcgggctcaacaaacgaggagagcatatcgaaaggtggacgatatgttgaaacagttaacagcggatcaaagtccttttctttgagaattaatgatctaacagttgaagacggtggcacgtatcgttgcggtgtctcgacgccctggtcgtgtgactacgggccgctgtgcttctatgctgcatgcggagatggcactgtcgtgactgtgaat

SEQ ID NO: 14 - DNA, 339 bp, synthetic construct
  misc_feature 1..339: note = Synthetic nucleic acid
  source 1..339: mol_type = other DNA
gctcgagtggaccaaacaccgcaaacaataacaaaggagacgggcgaatcactgaccatcaactgtgtcctacgagatagtaactgtccattgtccaacacctactggtatcgcaaaaaatcgggctcaacaaacgaggagagcatatcgaaaggtggacgatatgttgaaacagttaacagcggatcaaagtccttttctttgagaattaatgatctaacagttgaagacagtggcacgtatcgatgcaaggtccgaactcactggtcgatatgctgggagatgggactggagcccaactatgatgtatacggaggtggcactgtcgtgactgtgaat

SEQ ID NO: 15 - DNA, 392 bp, synthetic construct
  misc_feature 1..392: note = Synthetic nucleic acid
  source 1..392: mol_type = other DNA
gctcgagtggaccaaacaccgagatcagtaacaaaggagacgggcgaatcactgaccatcaactgtgtcctacgagatgcgagctatgcattgggcagcacgtgctggtatcgaaaaaaatcgggctcaacaaacgaggagagcatatcgaaaggtggacgatatgttgaaacagttaacagcggatcaaagtccttttctttgagaattaatgatctaacagttgaagacggtggcacgtatcgttgcggtcggatccaaaacacgaaagctgtgcttctcccatgtgtctatgctgcatgcggagatggcactgtcgtgactgtgaatagtggccaggccggccaccaccaccaccaccacggcgactacaaggacgatgacgataaggg

SEQ ID NO: 16 - DNA, 395 bp, synthetic construct
  misc_feature 1..395: note = Synthetic nucleic acid
  source 1..395: mol_type = other DNA
gctcgagtggaccaaacaccgagatcagtaacaaaggagacgggcgaatcactgaccatcaactgtgtcctacgagatgcgagctatgcattgggcagcacgtgctggtatcgaaaaaaatcgggctcaacaaacgaggagagcatatcgaaaggtggacgatatgttgaaacagttaacagcggatcaaagtccttttctttgagaattaatgatctaacagttgaagacggtggcacgtatcgttgcggtgtccaaagcaggtggggagctgtgcccaagctgtgctctaactatgctgcatgcggagatggcactgccgtgactgtgaatagtggccaggccggccaccaccaccaccaccacggcgactacaaggacgatgacgataaggg

SEQ ID NO: 17 - DNA, 333 bp, synthetic construct
  misc_feature 1..333: note = Synthetic nucleic acid
  source 1..333: mol_type = other DNA
gctcgagtggaccaaacaccgcaaacaataacaaaggagacgggcgaatcactgaccatcaactgtgtcctacgagatagtaactgtgcattgtccagcacgtactggtatcgcaaaaaatcgggctcaacaaacgaggagagcatatcgaaaggtggacgatatgttgaaacagttaacagcggatcaaagtccttttctttgagaattaatgatctaacagttgaagacagtggcacgtatcgatgcaaggtaggctgctggtacggctatgagtcgtcactcatgaactggagcagggtatacggaggtggcactgccgtgactgtgaat

SEQ ID NO: 18 - DNA, 321 bp, synthetic construct
  misc_feature 1..321: note = Synthetic nucleic acid
  source 1..321: mol_type = other DNA
gctcgagtggaccaaacaccgagatcagtaacaaaggagacgggcgaatcactgaccatcaactgtgtcctacgagatgcgagctatgcattgggcagcacgtgctggtatcgaaaaaaatcgggctcaacaaacgaggagagcatatcgaaaggtggacgatatgttgaaacagttaacagcggatcaaagtccttttctttgagaattaatgatctaacagttgaagacagtggcacgtatcgatgcaaggggtacacgccctgtatgcccgactgggtccccgatgtatacggaatgggcactgtcgtgactgtgaat

SEQ ID NO: 19 - DNA, 366 bp, synthetic construct
  misc_feature 1..366: note = Synthetic nucleic acid
  source 1..366: mol_type = other DNA
caggtgcagctggtggagtctggcggaggatccgtgcagccaggaggaagcctgaggctgtcttgcgccgtgtccggctacacctccagctcttattgtttcggctggtttagacaggctccaggcaaggacagggagggaatcatcgctatggagccaggaaacacctacacagctggagctgtgaagggcaggttcaccatcagccgggataacgccaagaatatggtgtatctgcagatgaactctctgaagcctgacgatacagctatgtactattgcgctgcttccccataccctggaacctggctgaatccagacctgtttgatacatggggccagggcacccaggtgacagtgtccagc

SEQ ID NO: 20 - DNA, 354 bp, synthetic construct
  misc_feature 1..354: note = Synthetic nucleic acid
  source 1..354: mol_type = other DNA
gccgtgcagctggtggagagcggcggaggatctgtgccacctggaggctccctgaccctgagctgcacagtgtctgacaacatcttccgcacctacgccgtgggctggtttcgccaggctccagagaagaggtgtcagtggatcgccaccctgaacgctgacgagagcacagagtacaatgtggccgtgaccggcaggttcacaatctctcgggataattctggcagatccgtgtatctgcagatggacaacctgaattctgaggatacagctatctactattgcgcctccctggctcggaagtggtcctgtcacgatagctggggcagaggcacccaggtgacagtgtccagc

SEQ ID NO: 21 - DNA, 372 bp, synthetic construct
  misc_feature 1..372: note = Synthetic nucleic acid
  source 1..372: mol_type = other DNA
gccgtgcagctagttgagagcggaggcggtcttgtccaaccaggagggagcctgagactgtcttgtgccgcatccggcttcacattctcatcctatgctatggggtgggtgcggcaggcccctggaaaaggtctggaatggatatccgcaatcaatagtggaggcgacattcgatattacgctgacagtgtcaagggcaggtttacaatctctcgcgacaatgcgaagaacaccctgtatttacaaatgaactctctcaaacccgaagataccgctgtgtactactgcgccactgatgatgagattttgtcgtacgggctcgggacctcatttgactattggggtcagggcactcaggtaacggtgtccagc

SEQ ID NO: 22 - DNA, 374 bp, synthetic construct
  misc_feature 1..374: note = Synthetic nucleic acid
  source 1..374: mol_type = other DNA
gccgtgcagcttgtggagagtggcgggggcctggtccaaccaggaggaagtctgcggctctcctgcgccgcctctgggttcacattttccagctatggaatgcactgggtaagacaggcgcccggtaaggggctcgaacgtgtggcaggtataaactgggatgggtctaattcttattacaccgagacggttaaaggccgcttcactatctcaagagacaacgctaagaatacagcttacctacaaatgaacagcctgaaacctgaagacaccgcaatgtactactgccgaacattaattttggactcggattgtgatccgggctcaacctataggtatgcctggggacagggcacccaggtcactgttccagc

SEQ ID NO: 23 - DNA, 384 bp, synthetic construct
  misc_feature 1..384: note = Synthetic nucleic acid
  source 1..384: mol_type = other DNA
gcggtccaattggtggatagcggcggcgggagcgtccaggctggagggagtctgaggttatcttgcgcaggaagcgcatttacgtacagttcctattgtatgggctggtttcgccaggcgccgggaaaggaaagagaggccgtggctgctatcaactccgggacaagctcaacttattacgctgattcagtgaagggtcggttcactgtttcgcaggataacgccaataataccgtatacctgctcatgatatctcttaaacccgaggacacagccatttatatctgcgccgccctggtggcccctgactatgacgactacgatgtcgcagcaccagacaacttcgattactggggtcagggcacccaagtgaccgtttccagc

SEQ ID NO: 24 - DNA, 360 bp, synthetic construct
  misc_feature 1..360: note = Synthetic nucleic acid
  source 1..360: mol_type = other DNA
gacgtccagttggtggagagtggcggcggagctgtgaaacccggaggatcgctcactctgacctgcaccgtgcaggaaggctcccgtaattgcgttggttggttccgccagggctccggtaaagatagacaagcagtggccatgattaacggcaatgggggtaccacaaactatgacgacagggtaaagggaagatttcagatcagcagggacggctctttagtccaccttcggatgaacgatgtcaagctgggtgatactgatatatactactgtcgacaggggcagacatatgattgttgggggggcacctggacggacagcggatgggggcaagggacacaggttactgtgtccagc

SEQ ID NO: 25 - protein, 8 aa, synthetic construct
  REGION 1..8: note = Synthetic polypeptide
NEESISKG

SEQ ID NO: 26 - protein, 5 aa, synthetic construct
  REGION 1..5: note = Synthetic polypeptide
NSGSK